(19) United States
(12) Patent Application Publication
(10) Pub. No.: US 2025/0325571 A1
(43) Pub. Date: Oct. 23, 2025

(54) TREATMENT OF BINGE EATING DISORDER USING PSYCHEDELICS

(71) Applicant: TRYP Therapeutics Inc., Kelowna (CA)

(72) Inventors: James GILLIGAN, Kelowna, British Columbia (CA); Peter GUZZO, Kelowna, British Columbia (CA)

(21) Appl. No.: 18/871,904

(22) PCT Filed: Jun. 8, 2023
(86) PCT No.: PCT/IB2023/055901

§ 371 Date: Dec. 5, 2024

Related U.S. Application Data
(60) Provisional application No. 63/350,393, filed on Jun. 8, 2022.
(60) Provisional application No. 63/437,347, filed on Jan. 5, 2023.

(51) Int. Cl. A61K 31/675; A61P 25/00

(52) U.S. Cl. CPC A61K 31/675; A61P 25/00

(57) ABSTRACT

Provided are methods for treating binge eating disorder (BED) or one or more symptoms thereof, that involve the administration of psychedelics, such as psilocybin, and related uses of the psychedelics. The disclosure in some aspects also relates to methods for identifying subjects for treatment of BED with administration of psychedelics. In some aspects, the treatment also involves psychotherapy. In some aspects, the subject receiving the treatment is assessed for various indicators, such as observer-rated and subject-reported outcomes, biological and clinical indicators, and combinations thereof.

### CROSS-REFERENCE TO RELATED APPLICATIONS

This application claims priority from U.S. provisional application No. 63/350,393, filed Jun. 8, 2022, entitled “TREATMENT OF BINGE EATING DISORDER USING PSYCHEDELICS”, and U.S. provisional application No. 63/437,347, filed Jan. 5, 2023, entitled “TREATMENT OF BINGE EATING DISORDER USING PSYCHEDELICS,” the contents of which are incorporated by reference in their entirety.

### FIELD

The present disclosure relates in some aspects to methods for treating binge eating disorder (BED) or one or more symptoms thereof, that involve the administration of psychedelics, such as psilocybin, and related uses of the psychedelics. The disclosure in some aspects also relates to methods for identifying subjects for treatment of BED with administration of psychedelics. In some aspects, the treatment also involves psychotherapy. In some aspects, the subject receiving the treatment is assessed for various indicators, such as observer-rated and subject-reported outcomes, biological and clinical indicators, and combinations thereof.

### BACKGROUND

Binge eating disorder (BED) is the most common eating disorder and is associated with obesity and psychiatric or behavioral comorbidities, including depression, anxiety, compulsive and impulsive behaviors. While medications and other approaches are available for managing BED, existing medications only provide a modest benefit and can be accompanied by side effects. Improved therapeutic approaches are needed. Provided are embodiments that meet such needs.

### SUMMARY

Provided herein are method of treating binge eating disorder (BED) that involves the administration of psychedelics, such as psilocybin or an active metabolite thereof, and uses of the psychedelics, such as psilocybin, in such treatment. In some aspects, the provided embodiments also involve psychotherapy or an active metabolite thereof.

Provided herein is a method of treating binge eating disorder (BED) that involves: orally administering to a subject suffering from one or more symptoms of BED, one or more doses of psilocybin or an active metabolite thereof effective to induce a dissociative state; providing one or more integration sessions to the subject after emergence from the dissociative state; and assessing in the subject one or more indicators prior to and/or after administration of the one or more doses of psilocybin or an active metabolite thereof. In some of any of the provided embodiments, the one or more indicators are associated with the one or more symptoms of BED, dissociative state, treatment outcome, safety and/or is related to the subject.

Provided herein is a method of treating binge eating disorder (BED) that involves assessing in the subject one or more indicators prior to and/or after administration of the one or more doses of psilocybin or an active metabolite thereof in a subject suffering from one or more symptoms of BED that has been provided one or more integration sessions after emergence from a dissociative state induced by orally administered one or more doses of psilocybin or an active metabolite thereof effective to induce a dissociative state. In some of any of the provided embodiments, one or more indicators associated with the one or more symptoms of BED, dissociative state, treatment outcome, safety and/or is related to the subject are assessed in the subject prior to and/or after administration of the one or more doses of psilocybin or an active metabolite thereof.

In some of any of the provided embodiments, the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).

Provided herein is a method of treating binge eating disorder (BED) that involves: orally administering to a subject suffering from one or more symptoms of BED, one or more doses of psilocybin or an active metabolite thereof effective to induce a dissociative state; providing one or more integration sessions to the subject after emergence from the dissociative state; and assessing in the subject one or more indicators prior to and/or after administration of the one or more doses of psilocybin or an active metabolite thereof. In some of any of the provided embodiments, the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).

Provided herein is a method of treating binge eating disorder (BED) that involves assessing in the subject one or more indicators prior to and/or after administration of the one or more doses of psilocybin or an active metabolite thereof in a subject suffering from one or more symptoms of BED that has been provided one or more integration sessions after emergence from a dissociative state induced by orally administered one or more doses of psilocybin or an active metabolite thereof effective to induce a dissociative state. In some of any of the provided embodiments, one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II) are assessed in the subject prior to and/or after administration of the one or more doses of psilocybin or an active metabolite thereof.

Provided herein is a method of treating binge eating disorder (BED) that involves: orally administering to a subject suffering from one or more symptoms of BED, one or more doses of psilocybin or an active metabolite thereof effective to induce a dissociative state; and providing one or more integration sessions to the subject after emergence from the dissociative state.

Provided herein is a method of treating binge eating disorder (BED) that involves providing one or more integration sessions a subject suffering from one or more symptoms of BED after emergence from a dissociative state induced by orally administered one or more doses of psilocybin or an active metabolite thereof effective to induce a dissociative state.

In some of any embodiments, the method also involves assessing in the subject one or more indicators prior to and/or after administration of the one or more doses of psilocybin or an active metabolite thereof.

In some of any embodiments, the one or more indicators are associated with the one or more symptoms of BED, dissociative state, treatment outcome, safety and/or is related to the subject.

In some of any embodiments, the method also involves, prior to administering the one or more doses of psilocybin or an active metabolite thereof, assessing one or more of the following parameters in the subject: (1) risk of suicide, (2) vital signs, (3) incidence of cardiovascular conditions, (4) incidence of gastrointestinal disease, (5) incidence of epilepsy, (6) incidence of schizophrenia spectrum or other psychotic disorders, (7) family history of psychosis, (8) a moderate or severe alcohol or drug use disorder, (9) prior adverse effects from psilocybin or an active metabolite thereof; and (10) use of prior or concomitant medications.

In some of any embodiments, the method also involves identifying the subject for treatment with psilocybin or an active metabolite thereof if the subject meets the criteria for the one or more parameters.

Provided herein is a method of identifying a subject for treating binge eating disorder (BED) with psilocybin or an active metabolite thereof that involves: assessing in a subject suffering from one or more symptoms of BED, one or more of the following parameters: (1) risk of suicide, (2) vital signs, (3) incidence of cardiovascular conditions, (4) incidence of gastrointestinal disease, (5) incidence of epilepsy, (6) incidence of schizophrenia spectrum or other psychotic disorders, (7) family history of psychosis, (8) a moderate or severe alcohol or drug use disorder, (9) prior adverse effects from psilocybin or an active metabolite thereof; and (10) use of prior or concomitant medications; identifying the subject for treatment with one or more doses of psilocybin or an active metabolite thereof if the subject meets the criteria for the one or more parameters.

Provided herein is a method of identifying a subject for treating binge eating disorder (BED) with one or more doses of psilocybin or an active metabolite thereof that involves identifying a subject suffering from one or more symptoms of BED for treatment with psilocybin or an active metabolite thereof if the subject meets the criteria for one or more of the following parameters that are assessed in the subject: (1) risk of suicide, (2) vital signs, (3) incidence of cardiovascular conditions, (4) incidence of gastrointestinal disease, (5) incidence of epilepsy, (6) incidence of schizophrenia spectrum or other psychotic disorders, (7) family history of psychosis, (8) a moderate or severe alcohol or drug use disorder, (9) prior adverse effects from psilocybin or an active metabolite thereof; and (10) use of prior or concomitant medications; identifying the subject for treatment with one or more doses of psilocybin or an active metabolite thereof if the subject meets the criteria for the one or more parameters.

In some of any embodiments, the method also involves orally administering to the identified subject, one or more doses of psilocybin or an active metabolite thereof effective to induce a dissociative state; and providing one or more integration sessions to the subject after emergence from the dissociative state.

In some of any embodiments, the method also involves assessing in the subject one or more indicators prior to and/or after administration of the one or more doses of psilocybin or an active metabolite thereof.

In some of any embodiments, the one or more indicators are associated with the one or more symptoms of BED, dissociative state, treatment outcome, safety and/or is related to the subject.

In some of any embodiments, the one or more indicators comprise observer-rated parameters, subject-reported parameters and/or clinical indicators.

In some of any embodiments, the one or more indicators are assessed prior to the administration as a baseline measurement, and after the administration as an outcome measurement. In some of any embodiments, the changes between the baseline measurement and the outcome measurement are determined.

In some of any embodiments, the subject exhibits amelioration of the one or more indicators between the baseline measurement and the outcome measurement.

In some of any embodiments, the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II). In some of any embodiments, the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, and body mass index (BMI).

In some of any embodiments, the one or more indicators include frequency of binge eating episodes. In some of any embodiments, the frequency of binge eating episodes is measured using the Eating Questionnaire. In some of any embodiments, the frequency of binge eating episodes is measured daily. In some of any embodiments, the one or more indicators include the number of binge eating episodes per day. In some of any embodiments, the frequency of binge eating episodes is determined over the 28 days prior to the date of measurement. In some of any embodiments, the one or more indicators comprises the frequency of binge eating episodes in the 28 days prior to the date of measurement. In some of any embodiments, the one or more indicators comprises frequency of binge eating desires as indicated by the sense of loss of control over eating. In some of any embodiments, the sense of loss of control over eating is measured using the Eating Questionnaire. In some of any embodiments, the sense of loss of control over eating is measured daily. In some of any embodiments, the one or more indicators comprises the frequency of sense of loss of control over eating per day. In some of any embodiments, the one or more indicators include interest in eating trigger foods. In some of any embodiments, the one or more indicators is measured using the Eating Questionnaire.

In some of any embodiments, the one or more indicators are selected from among Emotional Breakthrough Inventory (EBI), Acceptance and Action Questionnaire-II (AAQ-II), Patient Global Impression-Improvement (PGI-I), and Hospital Anxiety and Depression Scale (HADS). In some of any embodiments, the one or more indicators is Hospital Anxiety and Depression Scale (HADS)—anxiety. In some of any embodiments, the one or more indicators is Hospital Anxiety and Depression Scale (HADS)—depression.

In some of any embodiments, the one or more indicators are one or more neurophysiologic biomarkers selected from among Resting state functional connectivity (fed and fasted) by functional magnetic resonance imaging (fMRI), task-related functional activation and connectivity during a food cue reactivity task (fed and fasted) by fMRI, voxel-based morphometry (grey matter volume/structure) by fMRI, and resting electroencephalogram (EEG).

In some of any embodiments, the one or more indicators are one or more metabolic biomarkers selected from among ghrelin, leptin, adiponectin, insulin, glucose, and HOMA-IR (Homeostatic Model Assessment of Insulin Resistance).

In some of any embodiments, the one or more indicators selected from among Monitor Rating Scale (MRS), Mystical Experience Questionnaire (MEQ30), and Challenging Experiences Questionnaire (CEQ).

In some of any embodiments, the one or more indicators are measured during one or more of the integration sessions.

In some of any embodiments, the one or more indicators selected from among vital signs, blood chemistry and hematology tests, urinalysis, electrocardiograms (ECG), and Columbia-Suicide Severity Rating Scale (C-SSRS). In some of any embodiments, the blood chemistry and hematology tests comprise measuring the levels of one or more of Na⁺, K⁺, Cl⁻, HCO₃⁻, Ca⁺⁺, Mg⁺⁺, P, BUN, creatinine, glucose, total bilirubin, albumin, ALT, AST, GGT, CK, LDH, alkaline phosphatase, complete blood count, white cell differential count and platelet count. In some of any embodiments, the urinalysis comprises measuring one or more of pH, specific gravity, protein, occult blood, glucose, and ketones plus microscopic examination of sediment for RBC, WBC, epithelial cells, casts, crystals, and bacteria.

In some of any embodiments, the parameter is presence of gastrointestinal disease and the subject meets the criteria for the parameter if the subject does not have a gastrointestinal disease that could interfere with absorption of orally-administered psilocybin or an active metabolite thereof. In some of any embodiments, the parameter is incidence of epilepsy and the subject meets the criteria for the parameter if the subject does not have epilepsy. In some of any embodiments, the parameter is incidence of schizophrenia spectrum or other psychotic disorders and the subject meets the criteria for the parameter if the subject does not have schizophrenia spectrum or other psychotic disorder that meets the DSM-5 criteria, major depressive disorder with psychotic features, or Bipolar I or Bipolar II Disorder. In some of any embodiments, the parameter is family history of psychosis, and the subject meets the criteria for the parameter if the subject does not have a family history of psychosis. In some of any embodiments, the parameter is a moderate or severe alcohol or drug use disorder, and the subject meets the criteria for the parameter if the subject does not have a moderate or severe alcohol or drug use disorder that meets the DSM-5 criteria. In some of any embodiments, the parameter is prior adverse effects from psilocybin or an active metabolite thereof, and the subject meets the criteria for the parameter if the subject does not have a prior adverse effects from psilocybin or an active metabolite thereof. In some of any embodiments, the parameter is use of prior or concomitant medications, and the subject meets the criteria for the parameter if the subject is not using any of the following prior or concomitant medications: UGT1A9 inhibitors, 1A10 inhibitors, regorafenib, rifampicin, phenytoin, eltrombopag, mefenamic acid, diflunisal, niflumic acid, sorafenib, isavuconazole, deferasirox, Ginseng, aldehyde or alcohol dehydrogenase inhibitors, disulfiram, amphetamines, buprenorphine, benzodiazepines, cocaine, methamphetamines, Ecstasy (MDMA), morphine, methadone, oxycodone, marijuana, ethyl glucuronide, fentanyl, tramadol, synthetic cannabinoids (K2) regular use of psychoactive prescription medication, opioids, tramadol, or benzodiazepines; concomitant use of antidepressants; regular use of medications having a primary centrally-acting serotonergic effect, monoamine oxidase inhibitors (MAOIs), or selective serotonin reuptake inhibitors (SSRIs), or use of serotonin-acting dietary supplements, 5-hydroxy-tryptophan, St. John's wort,

In some of any embodiments, the subject is identified for treatment with psilocybin or an active metabolite thereof if the subject meets the criteria for all of the parameters (1)-(10).

In some of any embodiments, the one or more indicators are measured at about 4 weeks and at about 12 weeks following administration of the one or more doses of psilocybin or an active metabolite thereof. In some of any embodiments, the one or more indicators are measured at about 4 weeks, at about 8 weeks and at about 12 weeks following administration of the one or more doses of psilocybin or an active metabolite thereof. In some of any embodiments, the one or more indicators are measured at about 4 weeks following administration of the one or more doses of psilocybin or an active metabolite thereof.

In some of any embodiments, the subject receiving treatment shows improvement in one or more symptoms of BED or one or more symptoms of BED are ameliorated.

In some of any embodiments, the subject receiving treatment shows improvements selected from among one or more of: decreased frequency of binge eating episodes, decreased Binge Eating Scale (BES), decreased binge eating episodes per day, decreased frequency of sense of loss of control over eating per day, decreased anxiety around food, decreased depression around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased overall anxiety, decreased anxiety regarding food, improved vision of self, improved self-confidence, improvement in depression and feeling genuinely happy, lack of binge eating desires, weight loss, improved eating behavior, decreased Hospital Anxiety and Depression Scale (HADS), decreased HADS—anxiety, decreased HADS—depression, decreased Patient Global Impression (PGI-I), and decreased Acceptance and Action Questionnaire (AAQ-II). In some of any embodiments, the subject receiving treatment shows improvements selected from among one or more of: decreased frequency of binge eating episodes, decreased Binge Eating Scale (BES), decreased binge eating episodes per day, decreased anxiety around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased overall anxiety, decreased anxiety regarding food, improved vision of self, improved self-confidence, improvement in depression and feeling genuinely happy, lack of binge eating desires, weight loss, improved eating behavior, decreased Hospital Anxiety and Depression Scale (HADS), decreased Patient Global Impression (PGI-I), and decreased Acceptance and Action Questionnaire (AAQ-II). In some of any embodiments, the subject receiving treatment shows improvements selected from among one or more of: decreased frequency of binge eating episodes, decreased Binge Eating Scale (BES), decreased binge eating episodes per day, decreased anxiety around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased Hospital Anxiety and Depression Scale (HADS), decreased Patient Global Impression (PGI-I), and decreased Acceptance and Action Questionnaire (AAQ-II).

In some of any embodiments, the subject receiving treatment shows improvements selected from among one or more of decreased overall anxiety, decreased anxiety regarding food, improved vision of self, improved self-confidence, improvement in depression and feeling genuinely happy, lack of binge eating desires, weight loss, and improved eating behavior.

In some of any embodiments, the one or more doses of psilocybin or an active metabolite thereof comprises 1, 2, 3, 4, or 5 doses of psilocybin or an active metabolite thereof. In some of any embodiments, the one or more doses of psilocybin or an active metabolite thereof comprises 1 dose of psilocybin or an active metabolite thereof. In some of any embodiments, the one or more doses of psilocybin or an active metabolite thereof comprises 2 doses of psilocybin or an active metabolite thereof.

In some of any embodiments, the dose is between at or about 10 mg and at or about 50 mg of psilocybin or an active metabolite thereof. In some of any embodiments, the dose is at or about 25 mg of psilocybin or an active metabolite thereof.

In some of any embodiments, the one or more integration session comprises 1, 2, 3, 4, or 5 integration sessions. In some of any embodiments, the one or more integration session comprises 1 integration session. In some of any embodiments, the one or more integration session comprises 2 integration sessions.

In some of any embodiments, the integration session is provided at or about 1 day after administering the dose of psilocybin or an active metabolite thereof. In some of any embodiments, the methods also involve further comprising providing one or more further integration sessions. In some of any embodiments, the one or more further integration session is provided between about 6 days and at or about 9 days after administering the dose of psilocybin or an active metabolite thereof. In some of any embodiments, the one or more further integration session is provided at or about 7 days after administering the dose of psilocybin or an active metabolite thereof.

In some of any embodiments, the one or more doses of psilocybin or an active metabolite thereof includes a first dose of psilocybin or an active metabolite thereof. In some of any embodiments, the first dose is between at or about 10 mg and at or about 50 mg of psilocybin or an active metabolite thereof. In some of any embodiments, the first dose is at or about 25 mg of psilocybin or an active metabolite thereof.

In some of any embodiments, the one or more integration session includes a first integration session and a second integration session. In some of any embodiments, the first integration session is provided at or about 1 day after administering the first dose of psilocybin or an active metabolite thereof. In some of any embodiments, the second integration session is provided between about 6 days and at or about 9 days after administering the first dose of psilocybin or an active metabolite thereof. In some of any embodiments, the second integration session is provided at or about 7 days after administering the first dose of psilocybin or an active metabolite thereof.

In some of any embodiments, the one or more doses of psilocybin or an active metabolite thereof includes a second dose of psilocybin or an active metabolite thereof. In some of any embodiments, the methods also involve providing one or more further integration sessions after the second dose.

### BRIEF DESCRIPTION OF THE DRAWINGS

FIG. 1 shows an exemplary treatment and assessment schedule in accordance with the provided embodiments.

FIG. 2A (Q1) and FIG. 2B (Q2) provide the score over time for daily Eating Questionnaire Q1 and Q2 in a subject that has been administered psilocybin to treat binge eating disorder (BED).

FIG. 3A (Q1), FIG. 3B (Q2), and FIG. 3C (Q3) provide the changes of the score for daily Eating Questionnaire Q1-Q3 in five (5) subject that has been administered psilocybin to treat BED, before and after administration.

FIG. 4A (Hospital Anxiety and Depression Scale (HADS)—anxiety) and FIG. 4B (HADS—depression) shows the changes over time of the average HADS scores in five (5) subject that has been administered psilocybin to treat BED, for up to 4 weeks before administration and 14 weeks after administration.

FIG. 5A depicts a topographic map of eyes open/closed condition and testing session (pre- vs. post-treatment) on average spectral power within the delta spectral band (1-3 Hz).

FIG. 5B depicts nodes of the salience network (SN). FIG. 5C depicts (a) left middle frontal gyrus, (b) left supramarginal gyrus, and (c) left angular gyrus.

### DETAILED DESCRIPTION

Provided herein are methods of treating binge eating disorder (BED) or one or more symptoms of BED that involves the administration of psychedelics, such as psilocybin, in conjunction with psychotherapy, and related uses for the psychedelics. In some aspects, the methods and uses involve the co-administration of psilocybin, or an active metabolite thereof, and psychotherapy. In some aspects, the provided methods and uses also involve psychotherapy, for example, in one or more clinical sessions. In some aspects, the methods involve assessing the effect of treatment for example, based on the measurement of one or more indicators, including measures of treatment outcome, observer-rated and subject-reported outcomes, quantification of clinical indicators, and combinations thereof. In some aspects, the methods and uses results in one or more clinical improvement for an individual having BED.

In some embodiments, the provided methods involve assessing in the subject one or more indicators prior to and/or after administration of the one or more doses of psilocybin or an active metabolite thereof; wherein the one or more indicators are associated with the one or more symptoms of BED, dissociative state, treatment outcome, safety and/or is related to the subject.

For example, in some embodiments, the one or more indicators associated with the one or more symptoms of BED include, but are not limited to, frequency of binge eating episodes, Binge Eating Scale (BES) scale, binge eating episodes per day, anxiety around food, depression about eating behavior, depression about control of weight, Clinician Global Impression—Improvement (CGI-I) scale, interest in eating trigger foods, binge eating desires, eating behavior, Hospital Anxiety and Depression Scale (HADS), Patient Global Impression (PGI-I) scale, and Acceptance and Action Questionnaire (AAQ-II).

In some embodiments, the one or more indicators associated with a dissociative state include, but are not limited to, Monitor Rating Scale (MRS), Mystical Experience Questionnaire (MEQ30), Challenging Experiences Questionnaire (CEQ). In some embodiments, the one or more indicators associated with treatment outcome include, but are not limited to, Resting state functional connectivity (fed and fasted) by functional magnetic resonance imaging (fMRI), task-related functional activation and connectivity during a food cue reactivity task (fed and fasted) by fMRI, voxel-based morphometry (grey matter volume/structure) by fMRI, resting electroencephalogram (EEG) metabolic biomarkers selected from among ghrelin, leptin, adiponectin, insulin, glucose, and HOMA-IR (Homeostatic Model Assessment of Insulin Resistance).

In some embodiments, an indicator related to the subject involves any physical, emotional, or clinical parameter that characterizes features of the patient. These can be self-reported or can be determined by a care giver such as a medical provider or psychologist. In some embodiments, indicators related to the subject include, but are not limited to, observer-rated and subject-reported parameters, clinical indicators, and body and weight-related indicators. In some embodiments, indicators related to the subject include, but are not limited to, waist circumference, body mass index (BMI), overall anxiety, anxiety regarding food, vision of self, self-confidence, feeling genuinely happy, weight, vital signs, blood chemistry and hematology tests, urinalysis, electrocardiograms (ECG), and Columbia-Suicide Severity Rating Scale (C-SSRS).

Provided are methods and uses for treating an individual with BED that involves administering of one or more doses of psilocybin or an active metabolite thereof to the individual orally, providing an integration session on one or more occasion to the patient after emergence from the dissociative state.

In some embodiments, psilocybin or an active metabolite thereof and compositions comprising psilocybin or an active metabolite thereof are useful in a variety of therapeutic and prophylactic indications. For example, the compositions are useful in treating a disease, condition or disorder, such as BED, in a subject. Such methods and uses include therapeutic methods and uses, for example involving administration of the psychedelic to a subject having a disease, condition, or disorder, such as BED. In some embodiments, psilocybin or an active metabolite thereof is administered in an effective amount to effect treatment of the disease or disorder, e.g., BED. Uses include uses of the compositions in such methods and treatments, and in the preparation of a medicament in order to carry out such therapeutic methods. In some embodiments, the methods are carried out by administering the psilocybin or an active metabolite thereof to the subject having or suspected of having BED. In some embodiments, the methods thereby treat the disease, condition, or disorder, e.g., BED, in the subject.

Also provided herein are uses of psilocybin or an active metabolite thereof in such methods and treatments, and in the preparation of a medicament in order to carry out such methods. In some embodiments, the methods and uses thereby improve, ameliorate and/or treat BED or one or more symptoms thereof, in the subject. In some embodiments, the provided therapeutic methods and uses that involve the administration, such as intravenous administration, of psilocybin or an active metabolite thereof, to a subject having, exhibiting or suffering from one or more symptoms of BED, for example, in an amount that is sufficient to rapidly induce a dissociative state or a psychedelic state in the subject, and/or an effective amount to improve or ameliorate one or more symptoms of BED.

BED is a common eating disorder that is associated with obesity and psychiatric comorbidities, including psychological distress, depression and anxiety regarding food and eating. BED in some cases characterized by recurrent episodes of excessive food intake accompanied by a sense of loss of control and psychological distress. Existing treatment options for BED are limited. While medications and other approaches are available for managing BED, existing medications only provide a modest benefit and are accompanied by side effects. Improved therapeutic approaches for BED are needed. Provided are methods and uses that meet such needs, that include the administration of psilocybin or an active metabolite thereof to treat BED.

In some embodiments, the provided methods and uses result in the improvement of one of more symptoms, signs, or clinical feature of BED. In some embodiments, the method results in the improvement of one of more symptoms of BED, wherein the one or more symptom comprises frequent dieting and weight loss, hoarding of food, concealment of eating behavior and evidence of such behavior, such as by eating late at night, attribution of one's successes and failures to weight, avoiding social situations where food may be present, feeling depressed or anxious, rapid and unhealthy weight gain and/or weight loss, chronic erratic eating behavior. In some embodiments, one or more symptoms of BED is ameliorated or improved when an individual with BED is treated in accordance with the embodiments described herein.

In some aspects, the provided methods and uses are based on an observation in a clinical study that subjects with BED treated in accordance with an exemplary method as described herein, including administration of psilocybin or an active metabolite thereof in conjunction with psychotherapy, exhibited positive behavioral changes immediately following the integration session, which persisted for at least 4 weeks or 14 weeks after administration. Observed improvements included reduced overall depression and/or anxiety and depression and/or anxiety around food, reduced compulsion to eat, improved self-image and confidence, and being more content with oneself, and a weight loss at 4 weeks, without adverse events. A single administration of psilocybin or an active metabolite thereof in conjunction with psychotherapy, in accordance with the provided embodiments, was observed to achieve improvements, both in BED symptoms and quality of life, that were similar to possible improvements achievable by 12 months of extensive psychotherapy. As described herein, the magnitude of changes for most subjects in binge eating, anxiety, and depression were dramatic, typically only observable after much longer periods of evidence-based therapy. In addition, a durable and lasting therapeutic effect was observed even after administration of a single dose of the psychedelic, showing an improvement BED symptom improvement can last for 80 days or more after a single administration of psilocybin. Accordingly, the results described herein demonstrate the substantial effect of the methods and uses in accordance with the provided embodiments.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHOD OF TREATMENT AND USES OF PSILOCYBIN FOR BINGE-EATING DISORDER (BED)

In some embodiments, the methods and uses involve administering psilocybin or an active metabolite thereof and psychotherapy to treat a subject having BED.

In some aspects, psilocybin or an active metabolite thereof can alter neuronal connections, psilocybin or an active metabolite thereof can be considered for treatment of anxiety, depression and anxiety around food, compulsive and impulsive behaviors, self-mutilatory behaviors, and repetitive and intrusive thoughts, especially about food in people with BED. In some embodiments, the methods and uses involve administering psilocybin or an active metabolite thereof and psychotherapy to treat a subject diagnosed with BED. In some embodiments, the methods and uses involve administering psilocybin or an active metabolite thereof and psychotherapy to treat a subject who has displayed or experienced one or more symptoms of BED. In some embodiments, a method of treatment comprising psilocybin or an active metabolite thereof and psychotherapy improves or treats BED. In some embodiments, the severity of one or more symptoms of BED is reduced following administration of a method of treatment comprising psilocybin or an active metabolite thereof and psychotherapy. In some embodiments, one or more indicators, signs, symptoms, or behaviors of BED, including but not limited to frequency of binge eating episodes is/are reduced, delayed, and/or prevented following administration of a method of treatment comprising psilocybin or an active metabolite thereof and psychotherapy.

In some aspects, the embodiments provided herein include treatment of an individual that involve administering to the individual one or more therapeutically effective dose(s) of psilocybin or an active metabolite thereof. In some aspects, the methods and uses involve administration of psilocybin or an active metabolite thereof and psychological support or mental health support to the subject. In some aspects, the methods and uses involve administration of psilocybin or an active metabolite thereof and psychotherapy to a subject. In some aspects, the provided embodiments involve providing one or more sessions of psychological support prior to a first or an initial dose of psilocybin or an active metabolite thereof, such as one or more preparation session(s). In some aspects, the provided embodiments involve providing psychological support during the psilocybin or an active metabolite thereof administration session. In some aspects, the provided embodiments involve administering psilocybin or an active metabolite thereof to a subject in a controlled environment, wherein the subject is provided with psychological support. In some aspects, the provided embodiments involve one or more sessions of psychological support post-administration of psilocybin or an active metabolite thereof.

In some aspects, an individual receiving treatment in accordance with the provided embodiments is subject to one or more evaluation prior to, during, and after treatment. In some embodiments, the evaluation of the individual receiving treatment comprises physical measures of efficacy, observer-rated and subject-reported outcomes, quantification of clinical indicators, and combinations thereof. In some embodiments, the evaluation includes a measure of efficacy, wherein the measure of efficacy is a body and/or weight indicator, including but not limited to a change from baseline in body mass index (BMI), waist circumference, body weight. In some embodiments, the evaluation of the individual receiving treatment includes an observer-rated and/or subject-reported analysis, comprising an eating behavior assessment, a response to treatment assessment, a psychological evaluation, a psychedelic experience assessment, and/or a clinical activity assessment, including but not limited to a binge eating assessment, a binge eating scale (BES) assessment, a clinical global impression-improvement (CGI-I) scale assessment, a patient global impression-improvement (PGI-I) scale assessment, a hospital anxiety and depression scale (HADS) assessment, an emotional breakthrough inventory (EBI) assessment, an acceptance and action questionnaire (AAQ-II), a mystical experience questionnaire (MEQ30), a challenging experiences questionnaire (CEQ), and/or a monitor rating scale (MRS) assessment. In some embodiments, the evaluation of the individual receiving treatment includes a quantitative measure of neurological clinical indicators and metabolic biomarkers, including but not limited to brain morphology, brain activity, functional activation and/or functional connectivity, leptin levels, adiponectin levels, ghrelin levels, glucose levels, and insulin levels, and a homeostasis model assessment of insulin resistance.

### A. Bed

The provided embodiments relate to the treatment of binge eating disorder (BED), using a psychedelic and/or psychotherapy. In some aspects, the provided embodiments can result in the reduction, delay or prevention of one or more of indicators, signs or symptoms of BED, such as any one or more of indicators, signs or symptoms of BED, including any of those described herein. In some aspects, any one or more of these indicators, signs or symptoms can be ameliorated based on the methods and uses as provided herein.

BED is one of the most common eating disorders and is associated with obesity and psychiatric comorbidities, including depression (Guerdjikova et al., 2021, Contemporary Clinical Trials, 110, 106587). BED in some cases characterized by recurrent episodes of excessive food intake accompanied by a sense of loss of control and psychological distress but without the inappropriate compensatory weight loss behaviors of bulimia nervosa (Guerdjikova et al., 2021, Contemporary Clinical Trials, 110, 106587; McElroy et al., 2015, JAMA Psychiatry, 72, 236). In some aspects, BED is associated with abnormal neural responses to food, especially highly palatable foods (Citrome, 2019, CNS Spectr, 24(S1), 4-13; Donnelly et al., 2018, J. Eating Disorders, 6:3; Boswell et al., 2021, Clinical Therapeutics, 43). Individuals affected by BED can suffer from severe anxiety and have significant compulsive and impulsive behaviors, particularly around eating (Guerdjikova et al., 2019, Med Clin North Am, 103(4), 669-680; Samodien & Chellan, 2021, Front Neuroendocrinol, 60, 100871). Self-mutilatory behaviors, such as skin picking can be observed in BED, presumably associated with abnormal neuronal connectivity (Houazene et al., 2021, Behav Res Ther, 138, 103804). Therapeutic agents that influence reward and executive function systems are believed to have potential for treatment of BED (Boswell et al., 2021, Clinical Therapeutics, 43).

BED is characterized by recurrent binge eating episodes, which involves consumption of an amount of food that is greater than what most people would consume under similar circumstances, recurring episodes of eating large quantities of food and feeling unable to stop. In some cases, nearly 30% of people seeking weight loss treatments show signs of BED, and up to 3.5% of females and 2.0% of males will develop BED at some point in their lives, with nearly 4 million women and 2 million men in the United States. Existing treatments have not been effective.

Subjects with BED may experience a sense of lack of control over eating during binge episodes, significant psychological distress (e.g., shame, guilt) about binge eating, and display behavior to compensate for binging, such as purging, fasting, and exercising excessively. Commonly described symptoms of BED include frequent dieting and weight loss, hoarding of food, concealment of eating behavior and evidence of such behavior, such as by eating late at night, attribution of one's successes and failures to weight, avoiding social situations where food may be present, and feeling depressed or anxious. Binge eating can cause rapid and unhealthy changes in weight, e.g., weight gain and/or weight loss, and chronic erratic eating behavior. BED and symptoms associated with binge eating disorder may result in obesity. However, BED is not always associated with obesity. Although non-obese or normal weight subjects with BED may display similar binging behavior to obese BED subjects, normal weight individuals have been found to engage significantly more in weight control behaviors, such as eating less meals or snacks in a day; exercising, skipping meals, and avoiding certain foods (Goldschmidt et al. Obesity (Silver Spring). 2011; 19(7):1515-1518). Table 1 below shows DSM-IV and DSM-5 diagnostic criteria for BED (Berkman et al., Comparative Effectiveness Reviews, No. 160., Rockville (MD): Agency for Healthcare Research and Quality (US), 2015; ES-1). Exemplary indicators, signs, symptoms or behaviors associated with BED include a sense of a lack of control, rapid pace of eating, eating past fullness or satiety, hyperphagia, concealing eating behavior, frequency and intensity of self-conscious or judgmental thoughts, e.g., feelings of self-disgust, depression, anxiety, and/or guilt and shame, and use of inappropriate compensatory behavior, e.g., purging, fasting, and/or excessive exercise. In some aspects, the provided embodiments can result in the reduction, delay or prevention of one or more of indicators, signs or symptoms of BED, such as any one or more of indicators, signs or symptoms of BED, including any of those described herein.

In some embodiments, the provided methods and uses result in the improvement of one of more symptoms, signs, or clinical feature of BED, for example, binge eating episodes per day, anxiety around food, sense of loss of control over one's eating, depression about eating behavior, depression about control of weight, resultant weight gain, hyperleptinemia, hyperinsulinemia, rapid weight gain, obesity, decreased resting energy expenditure, reduced physical activity, with uncontrolled appetite, leptin resistance, deficient metabolism, fatigue, uncontrollable weight gain. In some embodiments, the methods result in the improvement of one of more symptoms of BED, for example, frequent dieting and weight loss, sense of loss of control over one's eating, hoarding of food, concealment of eating behavior and evidence of such behavior, such as by eating late at night, attribution of one's successes and failures to weight, avoiding social situations where food may be present, feeling depressed or anxious, rapid and unhealthy weight gain and/or weight loss, chronic erratic eating behavior. In some embodiments, the methods result in the improvement of one of more symptoms of BED, such as neurologic abnormalities, cognitive abnormalities, endocrine abnormalities, behavioral abnormalities, or obsessive-compulsive behavior. In some aspects, the provided methods and uses result in decreased overall anxiety, decreased anxiety regarding food, decreased depression, decreased depression regarding food, improved vision of self, improved self-confidence, improvement in depression and feeling genuinely happy, lack of binge eating desires, weight loss, and improved eating behavior.

TABLE 1
DSM-IV and DSM-5 diagnostic criteria
for Binge-Eating Disorder (BED)

| Criteria | |
|---|---|
| Set | Specific Definitions for Each Criterion |
| Criterion 1 | Recurrent episodes of binge eating. An episode of binge |
| | eating is characterized by both of the following: |
| | a. Eating, in a discrete period of time (e.g., within any |
| | 2-hour period), an amount of food that is definitely larger |
| | than most people would eat in a similar period of time under |
| | similar circumstances |
| | b. The sense of lack of control over eating during the |
| | episode (e.g., a feeling that one cannot stop eating or |
| | control what or how much one is eating) |
| Criterion 2 | Binge-eating episodes are associated with three (or more) |
| | of the following: |
| | a. Eating much more rapidly than normal |
| | b. Eating until feeling uncomfortably full |
| | c. Eating large amounts of food when not feeling |
| | physically hungry |
| | d. Eating alone because of being embarrassed by how |
| | much one is eating |
| | e. Feeling disgusted with oneself, depressed, or very guilty |
| | after overeating |
| Criterion 3 | Marked distress regarding binge eating is present. |
| Criterion 4 | The binge eating occurs, on average, |
| | a. at least 2 days a week for 6 months (DSM-IV frequency |
| | and duration criteria) |
| | b. at least 1 day a week for 3 months (DSM-5 frequency |
| | and duration criteria) |
| Criterion 5 | The binge eating is not associated with the regular use of |
| | inappropriate compensatory behavior (e.g., purging, fasting, |
| | excessive exercise) and does not occur exclusively |
| | during the course of anorexia nervosa or bulimia nervosa. |
| Criterion 6 | DSM-IV does not include BED severity grading scale. |
| | Applicable to DSM-5 only, BED severity is graded as follows: |
| | Mild: 1 to 3 episodes per week |
| | Moderate: 4 to 7 episodes per week |
| | Severe: 8 to 13 episodes per week |
| | Extreme: 14 or more episodes per week |

Certain treatment recommendations for BED are available (see, e.g., Association AP. American Psychiatric Association. Am J Psychiatry. 2006 July; 163(7 Suppl):4-54; Yager et al. American Psychiatric Association; 2012. p. 1-18; National Institute for Health and Care Excellence (NICE). 2004; Aigner et al., World J Biol Psychiatry. 2011; 12(6):400-43; Ozier and Henry, J Am Diet Assoc. 2011; 111(8):1236-41). Such treatment guidance generally involves the use of cognitive behavioral therapy and selective serotonin reuptake inhibitors (Berkman et al., Comparative Effectiveness Reviews, No. 160., Rockville (MD): Agency for Healthcare Research and Quality (US), 2015; 8-9). Additionally, lisdexamfetamine, second-generation antidepressants, and topiramate have been found to ameliorate BED in adults (Brownley et al., Ann Intern Med. 2016; 165(6): 409-420). However, available treatments have shown limited effect and/or has been associated with side effects.

In some aspects, psilocybin or an active metabolite thereof can be used to treat one or more behaviors or symptoms associated with BED, such as any described herein. For example, behaviors or symptoms to be treated in accordance with the described embodiments involving administration of psilocybin or an active metabolite thereof, include anxiety around food, perseveration, and repetitive and intrusive thoughts, especially around food. In some aspects, one or more indicators as described herein are associated with measuring or assessing one or more symptoms of BED, dissociative state, treatment outcome, safety and/or is related to the subject. In some aspects, the methods and uses provided herein include measuring or assessing one or more symptoms of BED before and then after administration of psilocybin or an active metabolite thereof.

### B. Administration of Psychedelics

In some aspects, the provided therapeutic methods and uses involve the administration of a psychedelic compound, such as psilocybin or a metabolite thereof, such as an active metabolite thereof. In some aspects, the administration of psychedelics is in conjunction with one or more clinical sessions, such as one or more preparation sessions or integration sessions, for psychotherapy. In some embodiments, the methods and uses involve orally administering one or more doses of psilocybin or an active metabolite thereof to a subject suffering from one or more symptoms of BED. In some aspects, if the subject is selected or identified for treatment with psilocybin or an active metabolite thereof, the subject is orally administered the one or more doses of psilocybin or an active metabolite thereof.

Psychedelics (“mind manifesting” drugs) are a class of compounds that have been used in the treatment of certain mental disorders (Nutt et al., Cell 2020; 181(1):24-8), including depression and addiction, anxiety and PTSD, and have shown a remarkable safety profile (REF).

Psilocybin (3-[2-(dimethylamino) ethyl]-1H-indol-4-yl]dihydrogen phosphate; 4-phosphoroyloxy-N,N-dimethyltryptamine) is a natural product produced by numerous species of Psilocybe mushrooms, which is manufactured for clinical use to control potency and purity. It is a tryptamine derivative, and in humans the phosphate group is rapidly enzymatically cleaved in the body to produce psilocin, an agonist at a variety of serotonin receptors, the most important of which in this setting is the 5-HT2A receptor (Carhart-Harris et al., 2014, Frontiers in Human Neuroscience, 8; Nichols, 2004, Pharmacol Ther, 101(2), 131-181). Psilocybin is a prodrug, and after administration, the phosphate group is rapidly cleaved in the body by alkaline phosphatase or other non-specific esterases to produce psilocin, the active metabolite (Dinis-Oliveira R, Drug Metab Rev 2017; 49(1):84-91). Psilocin is an agonist for multiple serotonin receptors, including the 5-hydroxytryptamine (5-HT)2A receptor (Carhart-Harris et al., Front Hum Neurosci 2014; 8:20; Nichols D, Pharmacol Ther 2004; 101(2):131-81). Psilocybin can induce profound changes in sensory perception, emotion, thought, and sense of self, characterized by marked alterations in all mental functions, including perception, mood, volition, cognition and self-experience (Geyer & Vollenweider, Trends Pharmacol Sci. 2008; 29(9):445-53; Studerus et al., J Psychopharmacol. 2011; 25(11):1434-52). These profound changes are often referred to as mystical-type experiences. Measures of mystical-type experience occurring during psilocybin treatment have been repeatedly observed to predict later effects on behavior and emotions, including reductions in depressive and anxious symptoms (Griffiths et al., J Psychopharmacol. 2016; 30(12):1181-1197; Maclean et al., J Psychopharmacol. 2011; 25(11):1453-1461; Ross et al., J Psychopharmacol. 2016; 30(12):1165-1180). Psilocybin has similar effects to dimethyltryptamine (DMT), lysergic acid diethylamide (LSD) and mescaline. These agents produce psychoactive effects that have been characterized as forming an intense dream-like state with colorful visual illusions, changes in auditory, tactile, olfactory, gustatory, and kinesthetic perceptions, altered perceptions of time and space, changes in body image, and sensations including ego dissolution, and intense mood changes ranging from feelings of wonder and bliss to sadness and grief.

In some aspects, psilocybin induces profound changes in sensory perception, emotion, thought, and sense of self, characterized by marked alterations in all mental functions, including perception, mood, volition, cognition and self-experience (Geyer & Vollenweider, Trends Pharmacol Sci. 2008; 29(9):445-53; Studerus et al., J Psychopharmacol. 2011; 25(11):1434-52). These profound changes are often referred to as mystical-type experiences which have been repeatedly observed to predict later effects on behavior and emotions, including reductions in depressive and anxious symptoms (Griffiths et al., J Psychopharmacol. 2016; 30(12):1181-1197; Maclean et al., J Psychopharmacol. 2011; 25(11):1453-1461; Ross et al., J Psychopharmacol. 2016; 30(12):1165-1180).

In some aspects, the action of psilocybin has been primarily associated with activation of the 5-HT2A receptor. The 5-HT2A receptor has been associated with hyperphagia in animal studies and in some human studies. Stimulation of 5-HT2A receptors in the paraventricular hypothalamus attenuates neuropeptide Y-induced hyperphagia through activation of corticotropin releasing factor (Grignaschi et al., 1996, Brain Res, 708(1-2), 173-176). The 5-HT2C/2B receptor agonist m-chlorophenylpiperazine inhibited 2-deoxy-D-glucose-induced hyperphagia in rats (Sugimoto et al., 2001, Biological and Pharmaceutical Bulletin, 24(12), 1431-1433). The association of 5-HT2A receptor gene polymorphism with eating disorders has also shown some conflicting results (Serretti et al., 2007, Curr Med Chem, 14(19), 2053-2069). Studies using positron emission tomography and single photon emission computed tomography with 5-HT-specific radioligands have consistently shown 5-HT(1A) and 5-HT(2A) receptor and 5-HT transporter alterations in anorexia nervosa and bulimia in cortical and limbic structures, which may be related to anxiety, behavioral inhibition, and body image distortions. In some aspects, being overweight was reported to be associated with increased 5-HT2A binding in most cortical regions in humans (Erritzoe et al., 2009, Neuroimage, 46(1):23-30).

Oral psilocybin has about a 50% bioavailability and psilocin is detectable in plasma within 20 minutes of administration of the parent compound (Brown et al. Clin Pharmacokinet. 2017; 56(12):1543-1554; Pharm Acta Helv. 1997; 72(3):175-84). The half-life of psilocin in blood is 2-3 hours. In general, onset of noticeable psychoactive effects occurs within one hour and peaks at about two hours after a dose. Loss of noticeable effects typically occurs around six hours after administration. Based on this time course, observation in the clinical trial setting until 8 hours after dosing. Furthermore, exposure following a 25 mg oral dose is associated with both near-maximal occupancy of neocortical serotonin 5-HT2A receptors and subjective intensity ratings of elements of the psychedelic experience that have been repeatedly associated with longer-term therapeutic benefits (Madsen et al., Neuropsychopharmacology. 2019; 44(7): 1328-1334).

Non-clinical studies have reported that, similar to humans, when psilocybin is administered orally to rats it is rapidly dephosphorylated to psilocin in the intestinal mucosa by alkaline phosphatase and a nonspecific esterase, with approximately 50% of the total volume of psilocin absorbed from the digestive tract (Kalberer et al., Biochem Pharmacol. 1962; 11:261-9). Maximum plasma levels are achieved after approximately 90 minutes (Chen et al., J Chromatogr B Analyt Technol Biomed Life Sci, 2011; 879(25):2669-72). When administered systemically (i.e., bypassing the gut), initial psilocybin metabolism is performed by tissue phosphatases, with in vitro studies indicating the kidneys as being among the most active metabolic organs (Horita & Weber, Biochem. Pharmacol. 1961; 7(1): 47-54). Across species tested, the highest levels of psilocin were found in the neocortex, hippocampus, and thalamus (Hopf & Eckert, Act Nerv Super (Praha) 1974; 16(1):64-6). In certain studies, psilocybin showed a decrease in symptomatic response in indications including obsessive compulsive disorder (OCD), substance use disorder, depression, and anxiety. Overall, psilocybin has been well tolerated at the doses examined in clinical studies.

Studies in rats demonstrated that psilocybin catabolism is similar to humans and approximately 50% of the total psilocin is absorbed from the digestive tract (Kalberer et al., Biochem Pharmacol 1962; 11:261-9), with maximum plasma levels achieved approximately 90 minutes post-administration (Chen et al., J Chromatogr B Analyt Technol Biomed Life Sci, 2011; 879(25):2669-72). In some aspects, when administered systemically (i.e., bypassing the gut), initial psilocybin metabolism is performed by tissue phosphatases, with in vitro studies indicating the kidneys as being among the most active metabolic organs (Horita & Weber, Biochem Pharmacol 1961; 7(1):47-54). Further, the highest levels of psilocin were consistently found in the neocortex, hippocampus, and thalamus (Hopf & Eckert, Act Nerv Super (Praha) 1974; 16(1):64-6).

In the clinical environment, psilocybin administration has been reported to improve certain symptoms of cancer-related psychiatric stress, depression and anxiety, nicotine and alcohol addiction, obsessive compulsive disorder (OCD) (Nutt et al., Cell 2020; 181(1):24-8), and anorexia nervosa, a condition where intrusive thoughts drive maladaptive and life-threatening behavior and in which treatment resistance is common (Foldi et al., 2020, Frontiers in Neuroscience, 14). Psilocybin was well tolerated at the doses examined in clinical studies. Further, a single dose of psilocybin was shown to improve patient-reported depression scores after 1 week, with these improvements persisting for up to 6 months (Carhart-Harris et al., Psychopharmacol 2018; 235(2):399-408). Psilocybin has since been given a fast-track designation by the FDA for depression. In any of the provided embodiments, the reference to administration of psilocybin can also includes administration of any active metabolites of psilocybin, such as psilocin.

### 1. Dosing

In some aspects, the methods and uses involve psilocybin dosing regimens to treat a subject having BED. In some aspects, provided herein are psilocybin dosing regimens to treat a patient or a subject diagnosed with, or is suffering from or exhibits one or more symptoms of BED. In some embodiments, a method for treating a subject comprises administering to the subject one or more therapeutically effective dose(s) of psilocybin.

In some embodiments, one or more doses of psilocybin is administered to the subject. In some embodiments, one, two, three, four or five doses of psilocybin is administered to the subject. In some embodiments, one dose of psilocybin is administered. In some embodiments, two or more doses of psilocybin is administered to the subject. In some aspects, two doses of psilocybin is administered.

In some embodiments, the non-initial, subsequent dose can be any dose of psilocybin that is administered after the initial or first dose, wherein at least two doses are administered to a subject. In some embodiments, the non-initial, subsequent dose can be any dose of psilocybin that is administered after the initial or first dose, wherein two or more doses of psilocybin are administered to a subject. In some embodiments, the non-initial subsequent dose can be a second, a third, or a fourth dose of psilocybin.

In some embodiments, when two or more doses of psilocybin is administered, the dosing interval between a previous dose and a subsequent dose, such as a first dose and a second dose, is at or about 1 week to 4 weeks. In some embodiments, a second dose of psilocybin is administered at or about 1 week, about 2 weeks, about 3 weeks, about 4 weeks, after the first dose of psilocybin. In some aspects, the dosing interval, for example the time between the first dose and the second dose, is at or about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, about 21 days, 22 days, 23 days, 24 days, 25 days, 26 days, 27 days, or 28 days. In some embodiments, a second dose of psilocybin is administered at or about at or about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, about 21 days, 22 days, 23 days, 24 days, 25 days, 26 days, 27 days, or 28 days after the first dose of psilocybin.

In some embodiments, a dose of psilocybin is administered to a subject, such as a subject having BED. In some aspects, the dose of the psilocybin is a therapeutically effective dose. In some aspects, the dose of psilocybin is effective to and/or sufficient to induce a dissociative state. In some aspects, the therapeutically effective dose is based on the route of administration.

In some embodiments, the dose of psilocybin is a flat or a fixed dose. In some embodiments, the dose of psilocybin to be administered is between at or about 5 mg and at or about 50 mg. In some embodiments, the dose of psilocybin to be administered is at or about 5 mg, 10 mg, 15 mg, 20 mg, 25 mg, 30 mg, 35 mg, 40 mg, 45 mg, or 50 mg. In some aspects, the dose of psilocybin is administered orally. In some aspects, for oral administration, the dose of psilocybin to be administered is between at or about 5 mg and at or about 50 mg, such as at or about 5 mg, 10 mg, 15 mg, 20 mg, 25 mg, 30 mg, 35 mg, 40 mg, 45 mg, or 50 mg. In some embodiments, the dose of psilocybin to be administered, such as by an oral administration, is at or about 25 mg. In some embodiments, the dose of psilocybin to be administered, such as by an oral administration, is at or about 50 mg.

In some aspects, one or more doses of psilocybin is administered. In some embodiments, the one or more doses are the same. In some embodiments, the one or more doses are different. In some embodiments, a first dose and a second dose is administered to the subject, and the first dose and the second dose are the same or substantially the same. In some embodiments, the first dose is greater than the second dose. In some embodiments, the second dose is greater than the first dose.

In some aspects, for oral administration, the first dose of psilocybin to be administered is between at or about 5 mg and at or about 50 mg, such as at or about 5 mg, 10 mg, 15 mg, 20 mg, 25 mg, 30 mg, 35 mg, 40 mg, 45 mg, or 50 mg. In some embodiments, the first dose of psilocybin to be administered, such as by an oral administration, is at or about 25 mg. In some embodiments, the first dose of psilocybin to be administered, such as by an oral administration, is at or about 50 mg.

In some embodiments, the dose determined according to body weight. In some embodiments, the first dose is determined according to body weight. In some aspects, exemplary dose based on body weight include between 0.1 mg/kg, at or about 0.15 mg/kg, at or about 0.25 mg/kg, at or about 0.35 mg/kg, at or about 0.45 mg/kg, at or about 0.55 mg/kg, at or about 0.65 mg/kg, at or about 0.75 mg/kg, at or about 0.85 mg/kg, at or about 0.95 mg/kg, or at or about 1.0 mg/kg. In some embodiments, a dose of at or about 25 mg of psilocybin is administered to a subject having a body weight of less than 100 kg (≤100 kg). In some embodiments, a dose of at or about 30 mg of psilocybin is administered to a subject having a body weight of greater than 100 kg and less than or equal to 117 kg (>100 kg and ≤117 kg). In some embodiments, a dose of at or about 35 mg of psilocybin is administered to a subject having a body weight of greater than 117 kg (>117 kg).

In some embodiments, a dose subsequent to the first dose of psilocybin is determined according to body weight. In some embodiments, the subsequent dose is increased by between at or about 0.1 mg/kg to at or about 1 mg/kg, such as at or about 0.1 mg/kg, at or about 0.15 mg/kg, at or about 0.25 mg/kg, at or about 0.35 mg/kg, at or about 0.45 mg/kg, at or about 0.55 mg/kg, at or about 0.65 mg/kg, at or about 0.75 mg/kg, at or about 0.85 mg/kg, at or about 0.95 mg/kg, or at or about 1.0 mg/kg, relative to the first, initial dose. In some embodiments, the subsequent dose is increased by at or about 0.15 mg/kg, at or about 0.25 mg/kg, at or about 0.35 mg/kg, at or about 0.45 mg/kg, at or about 0.55 mg/kg, at or about 0.65 mg/kg, at or about 0.75 mg/kg, at or about 0.85 mg/kg, and at or about 0.95 mg/kg relative to the first, initial dose.

In some embodiments, a subsequent dose of at or about 30 mg of psilocybin is administered to a subject having a body weight of less than 80 kg (≤80 kg). In some embodiments, a subsequent dose of at or about 35 mg of psilocybin is administered to a subject having a body weight of greater than 80 kg and less than or equal to 90 kg (>80 kg and ≤90 kg). In some embodiments, a subsequent dose of at or about 40 mg of psilocybin is administered to a subject having a body weight of greater than 90 kg and less than or equal to 100 kg (>90 kg and ≤100 kg). In some embodiments, a subsequent dose of at or about 45 mg of psilocybin is administered to a subject having a body weight of greater than 100 kg and less than or equal to 112 kg (>100 kg and ≤112 kg). In some embodiments, a subsequent dose of at or about 50 mg of psilocybin is administered to a subject having a body weight of greater than 112 kg (>112 kg).

In some aspects, psilocybin is administered orally. In some embodiments, psilocybin is administered in tablet form. In some embodiments, psilocybin is administered in capsule form. In some embodiments, one or more psilocybin 5 mg capsule(s) is administered to meet the desired dosing level in a subject. In some embodiments, one or more psilocybin 25 mg capsule(s) is administered to meet the desired dosing level in a subject. In some embodiments, psilocybin is administered capsules comprising one or more of gelatin, hydroxypropyl methyl cellulose (HPMC) or hypromellose, and pullulan. In some embodiments, psilocybin is administered capsules comprising hydroxypropyl methyl cellulose (HPMC) or hypromellose.

In some embodiments, wherein psilocybin is administered to a subject more than once, or repeatedly dosed, the amount of the initial dose and the amount of the one more subsequent dose(s) are the same. In some embodiments, wherein psilocybin is administered to a subject more than once, or repeatedly dosed, the amount of the initial dose and the amount of the one more subsequent dose(s) are different. In some embodiments, wherein psilocybin is administered to a subject more than once, or repeatedly dosed, the amount of the initial dose is less than the amount of the one more subsequent dose(s). In some embodiments, wherein psilocybin is administered to a subject more than once, or repeatedly dosed, the amount of the initial dose is greater than the amount of the one more subsequent dose(s).

In some embodiments, two doses of psilocybin are administered, wherein the first and the second dose are the same. In some embodiments, t two doses of psilocybin are administered, wherein the first and the second dose are different. In some embodiments, two doses of psilocybin are administered, wherein the first dose is equal in amount to the second dose. In some embodiments, t two doses of psilocybin are administered, wherein the first dose is lesser in amount relative to the second dose. In some embodiments, two doses of psilocybin are administered, wherein the first dose is lesser in amount relative to the second dose. In some embodiments, the two doses of psilocybin are administered, wherein the second dose is greater in amount relative to the first dose.

In some embodiments, the methods and uses also include providing psychological support and/or psychotherapy in addition to administration of psilocybin. In some aspects, the psychological support and/or psychotherapy is as described, for example, in Section I.C.

### C. Psychological Support

In some aspects, the provided embodiments involve providing psychological support or mental health support to a subject, in addition to or in conjunction with the administration of psilocybin or an active metabolite thereof. In some aspects, the provided embodiments involve administration of psilocybin or an active metabolite thereof and psychotherapy to a subject.

In some embodiments, psychological support comprises psychotherapy and/or talk therapy. In some embodiments, psychological support is therapist-led or led by a person or persons other than the subject. In some embodiments, psychological support is self-administered, for example, for accompanying administration of a subsequent dose or a maintenance dose. In some embodiments, psychological support is therapist-led and self-administered. In some aspects, the provided embodiments involve administration of psilocybin and talk therapy to a subject. In some embodiments, psychological support or mental health support occurs in-person and/or remotely, such as by phone or video conference. In some embodiments, psychotherapy occurs in-person and/or remotely, such as by phone or video conference. In some embodiments, talk therapy occurs in-person and/or remotely, such as by phone or video conference. In some embodiments, the psilocybin is self-administered, and the psychological support or mental health support occurs remotely, such as by phone or video conference.

In some embodiments, psychological support precedes administration of psilocybin, such as during a preparation session. In some embodiments, psychological support does not precede administration of psilocybin. In some embodiments, psychological support accompanies administration of psilocybin. In some embodiments, psychological support follows or occurs after administration of psilocybin, such as during an integration session.

In some aspects, the provided embodiments involve providing psychological support and/or psychotherapy in one or more sessions, such as one or more preparation sessions or integration sessions. In some embodiments, psychological support is facilitated in one or more sessions. In some embodiments, psychological support and/or psychotherapy is provided prior to administration of psilocybin, such as during a preparation session. In some embodiments, psychological support accompanies administration of psilocybin. In some embodiments, psychological support and/or psychotherapy is provided subsequent to administration of psilocybin, such as during an integration session. In some embodiments, psychological support and/or psychotherapy is provided prior to, during administration, and subsequent to administration of psilocybin, wherein psilocybin is administered at least once, at least twice, at least three times, or at least four times. An exemplary schedule of psychological support sessions in accordance with the provided embodiments is presented in Table 2.

TABLE 2
Exemplary Psychological Support Sessions

| Session | Setting* | Psychological Support Objectives |
|---|---|---|
| 1. Preparatory | In-person | Therapist aims to establish therapeutic |
| Session 1 (1 | or video | alliance through: |
| week prior | Lead | Building rapport and therapeutic alliance |
| to medication | therapist | Listening to participant's narrative of binge |
| session) | | eating, anxiety, guilt, etc., and treatment |
| | | history to understand patterns of |
| | | psychological inflexibility that are most |
| | | prominent |
| | | Psychoeducation regarding the psilocybin |
| | | experience and Acceptance and Commitment |
| | | Therapy (ACT) |
| 2. Preparatory | In-person | Teaching grounding techniques including |
| Session 2 | or video | abdominal breathing and dropping anchor |
| (1-3 days | Both | Discussion of therapeutic boundaries (e.g., |
| before | therapists | touch) and safety measures |
| medication | | Assisting the participant in setting a |
| session) | | motivation for medication session |
| 3. Medication | In-person | In line with supportive stance during the |
| session | Both | medication session, the therapist does not |
| (≥8 hrs) | therapists | provide significant ACT interventions or |
| | | feedback |
| | | ACT-based clinical formulation continues |
| | | as therapist listens to emergent narratives |
| | | and notes instances of psychological |
| | | flexibility and inflexibility, especially |
| | | present moment |
| | | awareness, avoidance, and values |
| 4. Integration | In-person | Therapist elicits complete narrative of |
| session 1 | Both | participant's experience during medication |
| (day after | therapists | session |
| medication | | Focused ACT-based clinical activity related |
| session) | | to contacting the present moment, defusion, |
| | | or acceptance based on clinical formulation |
| | | (time permitting). |
| 5. Integration | Video or | Therapist and participant continue to review |
| session 2 | In-person | and reflect on the participant's: |
| (~7 days after | Both or | psilocybin experience, including any |
| medication | one | emotional, mental, or lifestyle changes that |
| session) | therapist | followed the dosing session |
| | | Focused ACT-based clinical activity related |
| | | to contacting the present moment, defusion, |
| | | or acceptance based on clinical formulation. |
| 6. Therapy | Video or | Therapist continues to explore and reinforce: |
| follow-up | In-person | Insights gained from the psilocybin |
| session (~2 | Lead | experience while assessing for changes in |
| weeks after | therapist | psychological flexibility |
| medication | | How the dosing and therapy sessions brought |
| session) | | each ACT process to light |
| | | Successful behavioral changes and |
| | | committed actions taken |
| | | Mindfulness practices and other concrete |
| | | ways the study experience can be translated |
| | | into lasting changes |
| | | Therapist leads termination discussion and |
| | | plans for follow-up care for the participant. |

*One or more therapists or psychological support facilitators will provide the described therapy.

In some embodiments, psychological support and/or psychotherapy is provided conducted in single-subject sessions, wherein one subject meets with one or more therapist(s). In some embodiments, psychological support and/or psychotherapy is provided conducted in single-subject sessions, wherein one subject meets with two or more therapist(s) In some embodiments, psychological support and/or psychotherapy is provided conducted in group sessions, wherein more than one subject meets with one or more therapist(s). In some embodiments, one or more of the subject's family members or friends may be present at the pre-administration psychological support session(s). In some embodiments, the one or more psychological support sessions conducted prior to administration of psilocybin is conducted in person or remotely, such as by phone or video conference.

### 1. Psychological Support Prior to Psilocybin Administration

In some embodiments, one or more sessions of psychological support and/or psychotherapy is provided prior to a first or an initial dose of psilocybin, such as one or more preparation session(s). In some embodiments, two or more sessions of psychological support, such as three, four or five or more sessions of psychological support and/or psychotherapy, is provided prior to a first or an initial dose of psilocybin.

In some embodiments, the subject participates in at least one psychological support session before administration, e.g., pre-administration, of psilocybin. In some embodiments, a pre-administration psychological support session is provided at or about 1 month or less, such as at or about four weeks, three weeks, two weeks or one week prior to administration of psilocybin. In some embodiments, the subject participates in at least one psychological support session before administration, e.g., pre-administration, of psilocybin. In some embodiments, a pre-administration psychological support session is provided at or about 1 week or less, such as at or about seven days, six days, five days, four days, three days, two days, or one day prior to administration of psilocybin.

In some embodiments, one or more psychological support sessions is conducted prior to administration of psilocybin. In some aspects, two psychological support sessions are provided prior to administration of psilocybin. In some embodiments, two psychological support sessions are provided prior to a first psilocybin administration session, such as two preparation sessions. In some aspects, two psychological support sessions are provided prior to administration of psilocybin, with the first session at or about 1 week prior to administration of psilocybin and at or about 1 to 3 days prior to the administration of psilocybin, such as at or about 2 days prior to the administration of psilocybin.

In some embodiments, the one or more psychological support sessions conducted prior to administration of psilocybin is conducted in person or remotely, such as by phone or video conference.

In some embodiments, breathing exercises meant to promote calm and/or ease anxiety may be provided, demonstrated and/or practiced. In some embodiments, breathing exercise comprise instructing the subject to focus on their breath and/or sensations associated with the breath throughout the body. In one example the subject may be instructed to breathe in for a count of four, to hold their breath for a moment, and then to breathe out for a count of eight. In some embodiments, the therapist and subject may discuss the most helpful ways to support in case of emotional distress during the psilocybin session. In some embodiments, the therapist may assist the subject in setting a motivation for the psilocybin session. In some embodiments, the subject is given access (e.g., online access) to materials concerning the safety and mechanism of action of psilocybin

In some embodiments, psychological support and/or psychotherapy conducted prior to psilocybin administration comprises the pursuit of one or more goals and/or objectives. In some embodiments, the one or more goal(s) and/or objective(s) of the pre-administration session comprise(s) establishing a therapeutic alliance between subject and therapist, answering the subject's questions and addressing any concerns, demonstrating and practicing self-directed inquiry and experiential processing. In some embodiments, the pre-administration psychological support sessions comprises discussion of the reliable and/or the potential effects of psilocybin. In some embodiments, the pre-administration psychological support session comprises preparing the subject for psilocybin administration by practicing relevant therapeutic techniques to reduce avoidance and anxiety, eliciting relevant therapeutic goals, building rapport, and/or establishing therapeutic alliance. In some embodiments, the pre-administration psychological support session includes listening to the subject's narrative of binge eating, anxiety, guilt, treatment history and others, such as to understand patterns of psychological inflexibility that are most prominent. In some embodiments, the pre-administration psychological support session comprises psychoeducation regarding the psilocybin experience and Acceptance and Commitment Therapy (ACT). In some embodiments, at one or more, two or more, or three or more psychological support sessions are conducted prior to administration of psilocybin.

### 2. Psychological Support Accompanying Psilocybin Administration

In some embodiments, psychological support is provided during the psilocybin administration session. In some aspects, the provided embodiments involve administering psilocybin to a subject in a controlled environment, wherein the subject is provided with psychological support. In some aspects, the provided embodiments involve administering one or more dose(s) of psilocybin, e.g., an initial and one or more subsequent doses, or a first and a second dose, to the subject in the controlled environment, wherein the subject is provided with psychological support.

In some embodiments, a therapeutically effective dose of psilocybin is administered to the subject in the controlled environment, wherein the subject is provided with psychological support. In some embodiments, one or more dose(s) of psilocybin, e.g., an initial and one or more subsequent doses, or a first and a second dose, of psilocybin is administered to the subject in the controlled environment, wherein the subject is provided with psychological support. In some embodiments, a psychological support provider does not provide significant Acceptance and Commitment Therapy (ACT) interventions or feedback. In some embodiments, ACT-based clinical formulation continues as the psychological support provider listens to emergent narratives and notes instances of psychological flexibility and inflexibility, especially present moment awareness, avoidance, and values.

### 3. Psychological Support Subsequent to Psilocybin Administration

In some embodiments, one or more sessions of psychological support and/or psychotherapy is provided post-administration of one or more doses psilocybin, such as the first dose of psilocybin. In some embodiments, the one or more sessions of psychological support and/or psychotherapy is provided as one or more integration sessions. In some aspects, the one or more integration sessions to the subject after emergence from the dissociative state from the one or more doses of psilocybin.

In some embodiments, one or more integration sessions, such as two, three or four or more integration sessions is provided after each dose of psilocybin. In some embodiments, two integration sessions is provided after each dose of psilocybin. In some aspects, two integration sessions are provided after the first dose of psilocybin. In some aspects, two integration sessions are provided after a subsequent dose of psilocybin, such as a second dose of psilocybin. In some aspects, the integration sessions include psychological support, psychotherapy and/or talk therapy. In some embodiments, psychological support is therapist-led or led by a person or persons other than the subject. In some embodiments, psychological support is self-administered. In some embodiments, aspects of psychological support are therapist-led and self-administered

In some embodiments, two psychological support sessions, such as two integration sessions are provided subsequent to administration of a first dose and/or a subsequent dose of psilocybin. In some embodiments, the two integration sessions are provided within two weeks after administration of psilocybin. In some embodiments, the two integration sessions are provided within one week after administration of psilocybin. In some embodiments, the first of two integration sessions is provided at or about one day after the psilocybin administration session. In some embodiments the second of two integration sessions is provided about a week after the psilocybin session. In some embodiments the second of two integration sessions is provided between at or about 7 days and at or about 14 days after the psilocybin session. In some embodiments the second of two integration sessions is provided about 7 days after the psilocybin session. In some embodiments the second of two integration sessions is provided 14 days after the psilocybin session. In some embodiments the second of two integration sessions is provided about two weeks after the psilocybin session.

In some embodiments, the one or more post-administration sessions, or one or more integration sessions, comprises eliciting a complete narrative of the subject's experience during the psilocybin session. In some embodiments, the post-administration sessions, or integration sessions, comprises focused Acceptance and Commitment Therapy (ACT)-based clinical activity related to contacting the present moment, defusion, or acceptance based on clinical formulation. In some embodiments, the post-administration sessions, or integration sessions, comprises reviewing and reflecting upon the subject's psilocybin experience, including any emotional, mental, or lifestyle changes that followed the dosing session.

### 4. Set and Setting

In some aspects, the safety of subjects that are administered a dose of psilocybin is enhanced by testing psilocybin within a “set and setting” protocol, in view of the psychoactive nature of psilocybin (Lyons & Carhart-Harris, J Psychopharmacology 2018; 32(7):811-819), in some cases due to the psychoactive nature of psilocybin. In some aspects, “set” relates to one or more of the emotional, cognitive, behavioral state, mindset, and expectations of subjects prior to, such as immediately prior, to psilocybin administration. In some aspects, “set” is addressed by the therapist in the pre-dosing psychotherapy sessions. In some aspects, “setting” relates to the physical environment in which administration of psilocybin occurs. In some aspects, by addressing the set and setting of the experience, the risk of a subject reporting a distressing event or injuring themselves can be reduced. In some embodiments, this approach comprises three components: 1) psychological support prior to psilocybin administration, e.g., preparation, 2) administration, and 3) post-administration psychological support to integrate the classic hallucinogen experience, e.g., integration.

In some embodiments, prior to administration of psilocybin, subjects undergo pre-exposure preparation sessions comprising rapport building with the therapists who would be present during administration of psilocybin. In some embodiments, prior to administration of psilocybin, subjects undergo pre-exposure preparation sessions comprising identifying personal themes and struggles that might be especially likely to impact the session experience. In some embodiments, the administration of psilocybin itself is conducted by one therapist. In some embodiments, the administration of psilocybin itself is conducted by two therapists, such as a male and a female therapist, who are present throughout the session. In some embodiments, sessions are typically conducted in a room designed to be quiet, comfortable, and aesthetically pleasing. In some embodiments, subjects are encouraged to wear eyeshades and listen to a program of music through headphones during the administration of psilocybin to aid them in focusing their attention inward.

In some embodiments, a subject is supervised until an observer judges that the effects of psilocybin have completely subsided. In some embodiments, the subject can be discharged when meeting the discharge criteria. In some embodiments, one criterion must be met for a subject to be discharged. In some embodiments, more than one criterion must be met for a subject to be discharged. In some embodiments, all criteria must be met for a subject to be discharged. In some embodiments, discharge criteria comprise one or more of whether a responsible friend or family member is available to accompany the subject home, whether the subject's blood pressure and heart rate have returned to pre-drug levels or normal levels, whether the subject is deemed by supervisory personnel to be free of any acute drug effects, whether the subject believes they have returned to their psychological baseline, whether the observer(s) judge that it is safe to discharge the subject, and whether the subject expresses a readiness to go home.

### D. Exemplary Treatment Regimen

An exemplary treatment regimen, in accordance with the provided embodiments, is described. In an exemplary treatment regimen, subjects undergo screening, preparation therapy sessions, dosing, integration therapy sessions and follow up for 12 weeks following the dose of psilocybin. The total participation in the study is up to approximately 5 months.

During the psilocybin administration session, a single oral dose of 25 mg psilocybin capsule is administered. The session monitors are trained to reassure the subjects if they experience acute anxiety, agitation, paranoia, or panic.

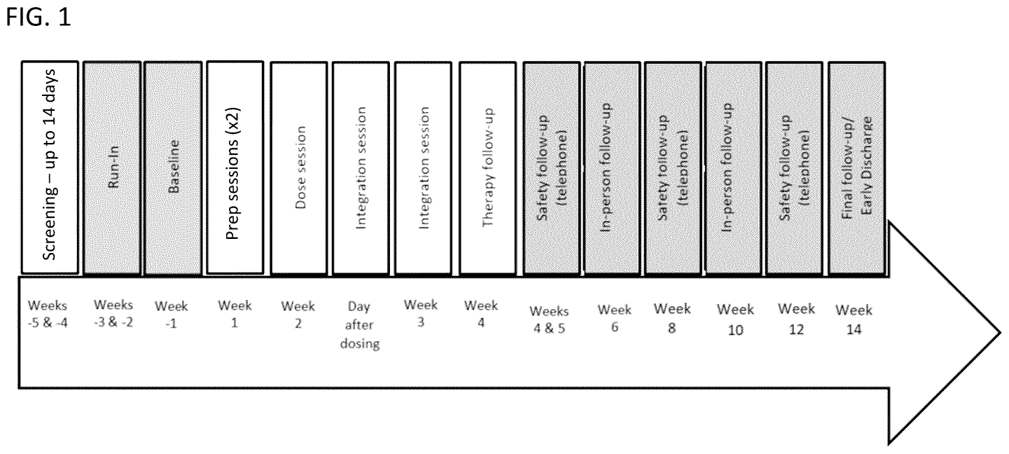

After the psilocybin administration session, subjects are engaged in in-person integration sessions the day after the dosing. Another integration session is held by video or in-person approximately 7 days after dosing. All therapists involved in this process are trained by an independent group, and the Psychotherapy Manual for all stages of the process are provided for subjects administered a psychedelic drug. At various time points after the administration session, follow-up sessions are provided to assess and evaluate one or more parameters or indicators as described In some aspects, such as in Section II. An exemplary treatment and assessment schedule in accordance with the provided embodiments is depicted in FIG. 1.

### II. MEASURES OF TREATMENT OUTCOME, OBSERVER-RATED AND SUBJECT-REPORTED PARAMETERS, AND CLINICAL INDICATORS

In some aspects, provided herein are methods and uses for psilocybin or an active metabolite thereof for treating binge eating disorder (BED) that involve measuring one or more parameters or indicators that are associated with or are indicative of one or more symptoms, signs or behaviors of BED, dissociative state, treatment outcome, safety and/or is related to the subject. In some aspects, an individual receiving treatment in accordance with the provided embodiments is subject to one or more evaluation prior to, during, and after treatment. In some aspects, the one or more indicators comprise observer-rated parameters, subject-reported parameters and/or clinical indicators.

In some embodiments, the evaluation of the individual receiving treatment includes measuring one or more physical measures of efficacy, observer-rated and subject-reported outcomes, quantification of clinical indicators, and combinations thereof. In some embodiments, the evaluation of the individual receiving treatment includes an observer-rated and/or subject-reported analysis, comprising an eating behavior assessment, a response to treatment assessment, a psychological evaluation, a psychedelic experience assessment, and/or a clinical activity assessment.

In some embodiments, the evaluation includes a measure of efficacy or treatment effect. In some aspects, the evaluation includes is a body and/or weight indicator, including but not limited to a change from baseline in body mass index (BMI), waist circumference, body weight, blood pressure, and blood lipid levels. In some embodiments, the evaluation of the individual receiving treatment includes an observer-rated and/or subject-reported analysis, for example, including an eating behavior assessment, a response to treatment assessment, a psychological evaluation, a psychedelic experience assessment, and/or a clinical activity assessment, including but not limited to a binge eating assessment, a binge eating scale (BES) assessment, lead therapist impressions, binge eating episodes per day, anxiety around food, depression about eating behavior, depression about control of weight, and/or a daily food diary, a clinical global impression-improvement (CGI-I) scale assessment, a patient global impression-improvement (PGI-I) scale assessment, a hospital anxiety and depression scale (HADS) assessment, an emotional breakthrough inventory (EBI) assessment, an acceptance and action questionnaire (AAQ-II), a mystical experience questionnaire (MEQ30), a challenging experiences questionnaire (CEQ), and/or a monitor rating scale (MRS) assessment. In some embodiments, the evaluation of the individual receiving treatment includes a quantitative measure of clinical indicators. In some embodiments, the evaluation of the individual receiving treatment includes a quantitative measure of neurological clinical indicators and metabolic biomarkers, including but not limited to brain morphology, brain activity, functional activation and/or functional connectivity, leptin levels, adiponectin levels, ghrelin levels, glucose levels, and insulin levels, and a homeostasis model assessment of insulin resistance. In some embodiments, the clinical indicators are related to the metabolism of an individual receiving treatment, including but not limited to hunger and satiety regulation, glucose metabolism, and glucose regulation via insulin.

In some aspects, due to the subjective nature of psychedelic intervention and general difficulty in objective quantification of patient outcomes after administration of psilocybin or an active metabolite thereof has been difficult. Exemplary approaches to quantify treatment outcomes and safety can include observer-rated and subject-reported outcomes, administered in the form of a time-sensitive survey.

In some aspects, one or more indicators, for example, that are associated with BED include frequency of binge eating episodes; Clinician Global Impression—Improvement (CGI-I) scale; waist circumference; and body mass index (BMI). In some aspects, the one or more indicators include interest in eating trigger foods. In some aspects, the one or more indicators include a binge eating scale (BES) assessment. In some aspects, the one or more indicators include a daily food diary.

In some aspects, the one or more indicators relate to the subject's response (e.g., change from baseline) to treatment and/or psychological status. psychedelic experience. In some aspects, the one or more indicators include an emotional breakthrough inventory (EBI), a mystical experience questionnaire (MEQ30), a challenging experiences questionnaire (CEQ), a monitor rating scale (MRS), hospital anxiety and depression scale (HADS), and/or acceptance and action questionnaire (AAQ-II).

In some aspects, an individual receiving treatment in accordance with the provided embodiments is subject to one or more evaluation prior to, during, and after treatment. In some embodiments, the evaluation of the individual receiving treatment comprises physical measures of treatment outcome, observer-rated and subject-reported outcomes, quantification of clinical indicators, and combinations thereof.

In some embodiments, the evaluation includes a measure of treatment outcome, wherein the measure of treatment outcome is a body and/or weight indicator. In some embodiments, the body and/or weight indicator is a change from baseline in body mass index. In some embodiments, the body and/or weight indicator is a change from baseline in waist circumference. In some embodiments, the body and/or weight indicator is a change from baseline in body weight. In some embodiments, the body and/or weight indicator is a change from baseline in blood lipid levels.

In some embodiments, the subject exhibits amelioration of the one or more indicators between the baseline measurement and the outcome measurement. In some aspects, the subject shows improvements or amelioration of any of the described indicators related to BED and/or related to the psychedelic experience. In some embodiments, the improvement is observed for a period of time after administration of a dose of the psychedelic, such as psilocybin or an active metabolite thereof. In some aspects, the improvement (e.g., therapeutic effect) is durable and lasting, even after a single administration of the psilocybin or an active metabolite thereof. In some embodiments, the improvement is observed for at least about 2 weeks, 4 weeks, 6 weeks, 8 weeks, 10 weeks, 12 weeks, 14 weeks and/or 16 weeks after the administration. In some embodiments, the improvement is observed for at least about 4 weeks. In some embodiments, the improvement is observed for at least about 8 weeks. In some embodiments, the improvement is observed for at least about 10 weeks. In some embodiments, the improvement is observed for at least about 12 weeks. In some embodiments, the improvement is observed for at least about 14 weeks. In some embodiments, the improvement is observed for at least about 16 weeks.

### A. Observer-Rated and Subject-Reported Parameters

In some aspects, one or more indicators include observer-rated and subject-reported parameters, for example, related to one or more symptoms or behaviors associated with BED, treatment outcomes and/or the experience related to administration of psilocybin or an active metabolite thereof, such as the psychedelic experience of the subject. In some aspects, one or more of the observer-rated and subject-reported parameters are indicative of treatment effects of the provided embodiments, e.g., resulting in improvement of one or more symptoms, signs or behaviors associated with BED, in accordance with the provided methods and uses.

### 1. Eating Behavior Assessments

Exemplary parameters related to eating behaviors and changes in eating behaviors are evaluated in an individual receiving treatment in accordance with the provided embodiments prior to, during, and after treatment. In some embodiments, such parameters include observer-rated and subject-reported outcomes.

a. Frequency of Binge Eating

The frequency of binge eating (FBE) evaluation is a three item patient reported survey derived from the Eating Questionnaire (Fairburn and Beglin, Int J Eat Disorder 1994; 16(4):363-70), which is used to quantify the frequency of binge eating events and similar behaviors having occurred in the past four weeks. Each item is answered with a numerical response that is summed and compared to a control and/or “normal” FBE score. In some embodiments, the change from baseline in the FBE is utilized to evaluate the treatment of BED. In some aspects, FBE is assessed based on the subject's answer to the following question (or a grammatical variant thereof): “Over the past 28 days, on how many DAYS have such episodes of overeating occurred (i.e., you have eaten an unusually large amount of food and have had a sense of loss of control at the time)?”

In some aspects, the embodiments provided herein include assessment of a change from baseline in frequency of binge eating (FBE) evaluation. In some aspects, the FBE evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the FBE score for an individual receiving treatment decreases during and/or after treatment, for example the FBE score decreases by at least 5 point, at least 10 points, at least 15 points, at least 20 points, at least 25 points, or at least 30 points compared to baseline. In some embodiments, the FBE for an individual receiving treatment decreases during and/or after treatment, for example the FBE decreases by at least at or about 50%, 55%, 60%, 65%, 70%, 75%, 80%, 85%, 90%, or 95% compared to the FBE at baseline. In some embodiments, the improvement in FBE is observed for at least about 2 weeks, 4 weeks, 6 weeks, 8 weeks, 10 weeks, 12 weeks, 14 weeks and/or 16 weeks after the administration. In some embodiments, the improvement in FBE is observed for at least about 4 weeks. In some embodiments, the improvement in FBE is observed for at least about 8 weeks. In some embodiments, the improvement in FBE is observed for at least about 10 weeks.

b. Binge Eating Scale (BES)

The binge eating scale (BES) is a 16-item questionnaire assessing the presence of certain binge eating behaviors which may be indicative of an eating disorder (Gormally et al., Addict Behav 1982; 7(1):47-55). Specifically, the BES evaluates both behavioral manifestations (e.g., eating large amounts of food) and thoughts/emotional states surrounding a binge episode (e.g., guilt, fear of being unable to stop eating). Previous work validated the BES evaluation, demonstrating response scores successfully discriminated among persons judged by trained interviewers to have either no, moderate or severe binge eating problems. Individuals receiving treatment are scored on a scale from 0-64, with 0 corresponding to no binge eating problems and 64 representing severe binge eating problems. In some embodiments, the BES is utilized to evaluate the treatment of BED.

In some aspects, the embodiments provided herein include assessment of a change from baseline in binge eating scale (BES) evaluation. In some aspects, the BES evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the BES score for an individual receiving treatment decreases during and/or after treatment, for example the BES score decreases by 1 or more points, 2 or more points, 3 or more points, 4 or more points, 5 or more points, 6 or more points, 7 or more points, 8 or more points, 9 or more points, 10 or more points, 12 or more points, 16 or more points, 20 or more points, 24 or more points, 36 or more points, 48 or more points, or 60 or more points.

c. Interest in Trigger Foods

In some aspects, the one or more indicators include interest in eating trigger foods, such as particular food items that trigger a binge eating episode or a desire for binge eating, of the trigger food or other food.

In some aspects, the embodiments provided herein include assessment of the subject's interest in eating trigger foods. In some aspects, the interest in eating trigger foods is evaluated at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

d. Binge Eating Episodes Per Day

In some aspects, the number of binge eating episode per day is evaluated in the subject. In some aspects, the number of binge eating episode per day is assessed prior to initiating the psychotherapy and/or prior to administration of the psilocybin. In some aspects, the number of binge eating episode per day is assessed both prior to initiating the psychotherapy and prior to administration of the psilocybin. In some aspects, the two different assessment of the number of binge eating episodes per day describes historical binge eating behavior and recent binge eating behavior just prior to initiating the methods and uses according to the provided embodiments. In some aspects, the change from baseline in the number of binge eating episode per day is utilized to evaluate the treatment of BED. In some aspects, the number of binge eating episodes per day is assessed based on the subject's answer to the following question (or a grammatical variant thereof): “Over the past 24 hours, how many times have you eaten what other people would regard as an unusually large amount of food (given the circumstances)?”

In some aspects, the embodiments provided herein include assessment of a change from baseline in number of binge eating episode per day. In some aspects, the number of binge eating episode per day is evaluated at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the number of binge eating episode per day score for an individual receiving treatment decreases during and/or after treatment. In some embodiments, the number of binge eating episode per day score for an individual receiving treatment decreases during and/or after treatment, for example the number of binge eating episode per day score decreases by at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8 1.9, 2.0, 2.1, 2.2, 2.3, 2.4, 2.5, 2.6, 2.7, 2.8, 2.9 or 3.0. In some embodiments, the number of binge eating episode per day score for an individual receiving treatment decreases during and/or after treatment, for example the number of binge eating episode per day score decreases by at least at or about 50%, 55%, 60%, 65%, 70%, 75%, 80%, 85%, 90%, or 95% compared to the number of binge eating episode per day score at baseline. In some embodiments, the number of binge eating episode per day score decreases by at least at or about 70% compared to the number of binge eating episode per day score at baseline. In some embodiments, the number of binge eating episode per day score decreases by at least at or about 80% compared to the number of binge eating episode per day score at baseline.

e. Loss of Control and Binge Eating Desires

In some embodiments, the one or more indicators comprises the frequency of sense of loss of control regarding one's eating. In some embodiments, the frequency of sense of loss of control regarding one's eating is indicative of or associated with binge eating desires. In some embodiments, the frequency of sense of loss of control regarding one's eating is measured using the Eating Questionnaire. In some embodiments, the frequency of sense of loss of control regarding one's eating is measured daily. The number of times a subject has a sense of loss of control regarding eating per day (loss of control score) is evaluated in the subject. In some aspects, the loss of control per day is assessed prior to initiating the psychotherapy and/or prior to administration of the psilocybin. In some aspects, the loss of control per day is assessed both prior to initiating the psychotherapy and prior to administration of the psilocybin. In some aspects, the two different assessment of the loss of control per day describes historical sense of loss of control and recent sense of loss of control just prior to initiating the methods and uses according to the provided embodiments. In some aspects, the change from baseline in the loss of control per day is utilized to evaluate the treatment of BED. In some aspects, the number of times a subject has a sense of loss of control regarding eating per day is assessed based on the subject's answer to the following question (or a grammatical variant thereof): “On how many of these times did you have a sense of having lost control over your eating (at the time that you were eating)?”

In some aspects, the embodiments provided herein include assessment of a change from baseline in loss of control per day. In some aspects, the loss of control per day is evaluated at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the loss of control per day score for an individual receiving treatment decreases during and/or after treatment. In some embodiments, the loss of control per day score for an individual receiving treatment decreases during and/or after treatment, for example the loss of control per day score decreases by at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8 1.9, 2.0, 2.1, 2.2, 2.3, 2.4, 2.5, 2.6, 2.7, 2.8, 2.9 or 3.0. In some embodiments, the loss of control per day score for an individual receiving treatment decreases during and/or after treatment, for example the loss of control per day score decreases by at least at or about 50%, 55%, 60%, 65%, 70%, 75%, 80%, 85%, 90%, or 95% compared to the loss of control per day score at baseline. In some embodiments, the loss of control per day score decreases by at least at or about 70% compared to the loss of control per day score at baseline. In some embodiments, the loss of control per day score decreases by at least at or about 80% compared to the loss of control per day score at baseline.

### 2. Response to Treatment Assessments and Psychological Evaluations

In some aspects, to evaluate an individual receiving treatment according to the provided embodiments, the individual is subject to one or more evaluation to determine the response (e.g., change from baseline) to treatment and/or psychological status. In some embodiments, the evaluation to determine the response to treatment and/or psychological status includes, for example, the clinical global impression-improvement (CGI-I) evaluation, the patient global impression-improvement (PGI-I) evaluation, the hospital anxiety and depression scale (HADS) evaluation, and/or the emotional breakthrough inventory (EBI) evaluation.

In some embodiments, one or more evaluation is completed one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

a. Clinical Global Impression-Improvement Scale (CGI-I)

The clinical global impression-improvement (CGI-I) evaluation is a clinician-rated change in the medical condition of an individual receiving treatment, wherein the individual is compared to baseline conditions, sometimes within the past seven days (Guy W., Assessment Manual for Psychopharmacology (revised) 1976:217-21). Individuals receiving treatment are rated on a scale from 1-7 for baseline conditions, wherein 1 is not ill at all and 7 is among the most extremely ill patients. Individuals receiving treatment are then rated on a scale from 1-7 for current condition, wherein 1 is “very much improved” since baseline (initiation of treatment), 4 is no change from baseline, and 7 is “very much worse” from baseline. In some embodiments, the change from baseline in CGI-I scale is utilized to evaluate the treatment of BED.

In some embodiments, the method provided herein includes a change from baseline in clinical global impression-improvement (CGI-I) evaluation. In some aspects, the CGI-I evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the CGI-I score for an individual receiving treatment improves during and/or after treatment, for example the CGI-I score decreases 1 or more points, 2 or more points, 3 or more points, 4 or more points, or 5-7 points. In some aspects, CGI-I related improvement involves decreasing the baseline conditions score and decreasing the current condition score.

b. Patient Global Impression-Improvement (PGI-I)

Similar to the CGI-I, the patient global impression-improvement (PGI-I) is a patient reported measure that reflects a patient's belief regarding the efficacy of treatment (Guy W., Assessment Manual for Psychopharmacology (revised) 1976:217-21). Individuals receiving treatment rate themselves on a scale from 1-7 for baseline conditions, wherein 1 is not ill at all and 7 is among the most extreme illness experienced. Individuals receiving treatment then rate their current condition on a scale from 1-7, wherein 1 is very much improved since baseline (initiation of treatment), 4 is no change from baseline, and 7 is very much worse from baseline. In some embodiments, the PGI-I scale is utilized to evaluate the treatment of BED.

In some embodiments, the method provided herein includes a patient global impression-improvement (PGI-I) evaluation. In some aspects, the PGI-I evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the PGI-I score for an individual receiving treatment decreases during and/or after treatment, for example the PGI-I score decreases by 1 or more points, 2 or more points, 3 or more points, 4 or more points, or 5-7 points. In some aspects, PGI-I related improvement includes decreasing the current condition score compared to the baseline. In some aspects, the PGI-I score is improved by at least at or about 1, 2, or 3 points compared to the baseline.

c. Hospital Anxiety and Depression Scale (HADS)

The hospital anxiety and depression scale (HADS) evaluation aims to measure psychological distress, symptoms of anxiety and depression using a 14 item questionnaire, wherein seven items are designated for the depression subscale (HADS Depression) and seven items are designated for the anxiety subscale (HADS Anxiety) (Zigmond and Snaith, Acta Psychiatr Scand 1983; 67(6):361-70). The depression branch focuses specifically on anhedonia, or the ability to feel pleasure, while the anxiety branch addresses general symptoms common to many anxiety disorders. As a means of evaluation, each HADS item is scored on a four option response-scale ranging between 0 and 3. Responses are then summed to obtain a total score for each two subscales, wherein an individual can be assessed as normal 0-7, borderline abnormal (case) 8-10, or abnormal (case) 11-21. Interpretation using a cut-off score of 8 or above for both HADS Anxiety and HADS Depression subscales is generally regarded as best practice.

In some aspects, the embodiments provided herein include assessment of a change from baseline in hospital anxiety and depression scale (HADS) evaluation. In some aspects, the HADS evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the HADS is utilized to evaluate the treatment of BED. In some embodiments, the HADS score for an individual receiving treatment decreases during and/or after treatment, for example the HADS score decreases by at least at or about 1 point, 2 points, 3 points, 4 points, 5 points, 6 points, 7 points, or 8 points, or more. In some embodiments, the HADS score for an individual receiving treatment decreases during and/or after treatment, for example the HADS score decreases by at least at or about 5%, 10%, 15%, 20%, 25%, 30%, 35%, 40%, 45%, 50%, 55%, 60%, 65%, 70%, 75%, 80%, 85%, 90%, or 95% compared to the HADS score at baseline. In some embodiments, the HADS score decreases by at least at or about 50% compared to the HADS score at baseline.

In some embodiments, the HADS anxiety score is utilized to evaluate the treatment of BED. In some embodiments, the HADS anxiety score for an individual receiving treatment decreases during and/or after treatment, for example the HADS anxiety score decreases by at least at or about 1 point, 2 points, 3 points, 4 points, 5 points, 6 points, 7 points, or 8 points, or more. In some embodiments, the HADS anxiety score for an individual receiving treatment decreases during and/or after treatment, for example the HADS anxiety score decreases by at least at or about 5%, 10%, 15%, 20%, 25%, 30%, 35%, 40%, 45%, 50%, 55% 60%, 65%, 70%, 75%, 80%, 85%, 90%, or 95% compared to the HADS anxiety score at baseline. In some embodiments, the HADS anxiety score decreases by at least at or about 50% compared to the HADS anxiety score at baseline. In some aspects, the improvement is observed for at least about 2 weeks, 4 weeks, 6 weeks, 8 weeks, 10 weeks, 12 weeks, 14 weeks and/or 16 weeks after the administration.

In some embodiments, the HADS depression score is utilized to evaluate the treatment of BED. In some embodiments, the HADS depression score for an individual receiving treatment decreases during and/or after treatment, for example the HADS depression score decreases by at least at or about 1 point, 2 points, 3 points, 4 points, 5 points, 6 points, 7 points, or 8 points, or more. In some embodiments, the HADS depression score for an individual receiving treatment decreases during and/or after treatment, for example the HADS depression score decreases by at least at or about 5%, 10%, 15%, 20%, 25%, 30%, 35%, 40%, 45%, 50%, 55%, 60%, 65%, 70%, 75%, 80%, 85%, 90%, or 95% compared to the HADS depression score at baseline. In some embodiments, the HADS depression score decreases by at least at or about 50% compared to the HADS depression score at baseline. In some aspects, the improvement is observed for at least about 2 weeks, 4 weeks, 6 weeks, 8 weeks, 10 weeks, 12 weeks, 14 weeks and/or 16 weeks after the administration.

d. Acceptance and Action Questionnaire (AAQ-II)

The acceptance and action questionnaire (AAQ-II) evaluation is a measure of psychosocial flexibility, defined broadly as the ability to fully contact the present moment and the thoughts and feelings it contains without needless defense (Hayes et al., The Psychological Record 2004; 54:553-78; Bond et al., Behav Ther 2011; 42(4):676-88). There are seven items included in the AAQ-II evaluation, and each response carries a numerical value, ranging from 1-7, wherein 1 is never true, 4 is sometimes true, and 7 is always true. Responses for each item are summed and the total score can be interpreted to determine the psychological flexibility of an individual, noting that a range of 24-28 is generally associated with depression, anxiety and/or related symptoms. In some embodiments, the AAQ-II is utilized to evaluate the treatment of one or more symptoms, signs or behaviors associated with BED, such as depression, anxiety and/or related symptoms associated with or related to food or eating.

In some aspects, the embodiments provided herein include assessment of a change from baseline in acceptance and action questionnaire (AAQ-II) evaluation. In some aspects, the AAQ-II evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the AAQ-II score for an individual receiving treatment decreases during and/or after treatment, for example the AAQ-II score decreases by at least at or about 1 point, 2 points, 3 points, 4 points, 5 points, 6 points, 7 points, 8 points, 9 points, 10 points, 15 points, 20 points, 25 points, 30 points, 35 points, 40 points, or 45 points.

### 3. Psychedelic Experience and Clinical Activity Assessments

In some aspects, to evaluate the psychedelic experience and the associated clinical impacts for an individual receiving treatment according to the provided embodiments, the individual is subject to a one or more evaluation to assess the psychedelic experience and clinical activity in response to treatment.

In some aspects, the one or more parameters or indicators related to the psychedelic experience can measure or assess the dissociative effects and status of the dissociative state in subjects with BED during or after administration of the psilocybin or an active metabolite thereof. In some aspects, the relationship between clinical activity or treatment outcome and the intensity of the psychedelic experience (e.g., as measured by one or more parameters or indicators related to the psychedelic experience) can be assessed.

In some embodiments, the evaluation to assess the psychedelic experience and clinical activity in response to treatment includes, for example, the mystical experience questionnaire (MEQ30) evaluation, the challenging experience questionnaire (CEQ) evaluation, and/or the monitor rating scale (MRS) questionnaire. In some embodiments, one or more psychedelic evaluation is completed at one or more time point, such as at or about 1 day after administration of one or more doses of psilocybin or an active metabolite thereof.

a. Mystical Experience Questionnaire (MEQ30)

The mystical experience questionnaire (MEQ or MEQ30; derived and abbreviated from the MEQ43) is an evaluation completed by the patient, comprising four factors: mystical (e.g., internal unity, external unity, noetic quality, and sacredness scales), positive mood, transcendence of time and space, and ineffability (Barrett et al., J Psychopharmacol 2015; 29(11):1182-90). The MEQ30 predicts persisting therapeutic benefits (e.g., change in attitudes, behavior, and well-being) attributed to psychedelic experiences, serving as a key measure of an individual's mystical experience. There are 30 items included in the MEQ30 evaluation, distributed across the four factors, and each items is rated on a 0-5 point scale, wherein 0 is “none; not at all,” 1 is “so slight cannot decide,” 2 is “slight,” 3 is “moderate,” 4 is “strong (equivalent in degree to any previous strong experience or expectation of this description),” and 5 is “extreme (more than ever before in my life and stronger than 4).” Across these 30 items, the four factors, or sub-categories, comprise different items, wherein mystical consists of items 4-6, 9 14-16, 18, 20, 21, 23-26, and 28; positive mood consists of items 2, 8, 12, 17, 27, and 30; space/time consists of items 1, 7, 11, 13, 19, and 22; and ineffability consists of items 3, 10, and 29. Scores for each item within a sub-category, or across the full MEQ30 set, are summed together and converted to a percentage by dividing by the corresponding point total. A “complete experience” is defined by a MEQ30 score equal to or exceeding 60% (of the total) across all four sub-categories, wherein the complete experience correlates with more significant and/or extended therapeutic benefits.

In some aspects, the embodiments provided herein include assessment of a change from baseline in the MEQ30 evaluation. In some aspects, the MEQ30 evaluation is completed at one or more time point, such as at 1 day post administration of one or more doses of psilocybin.

b. Emotional Breakthrough Inventory (EBI)

The emotional breakthrough inventory (EBI) is a six-item evaluation assessing the emotional breakthrough during a psychedelic experience, and is considered an important component and key mediator of long term psychological changes (Roseman et al., J Psychopharmacol 2019; 33(9):1076-87). The EBI scores appear to be dose-dependent, as may be predicted, and is complimentary with other well-established and commonly used evaluations, such as the mystical experience questionnaire (MEQ) and the challenging experiences questionnaire (CEQ). Items within the EBI evaluation are rated using a visual analogue scale (VAS) (0-100, with incremental units of one) with 0 defined as ‘No, not more than usually’, and 100 defined as ‘Yes, entirely or completely.’

In some aspects, the embodiments provided herein include assessment of a change from baseline in emotional breakthrough inventory (EBI) evaluation. In some aspects, the EBI evaluation is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

c. Challenging Experiences Questionnaire (CEQ)

Due to the powerful experience induced by classical hallucinogens (e.g., psilocybin), a patient receiving treatment may endure one or more acute adverse psychological reactions (i.e., a bad trip, or a challenging experience). Case reports and anecdotal evidence suggests symptoms including affective (panic, depressed mood), cognitive (confusion, feelings of losing sanity), and somatic (nausea, heart palpitation) are possible (Barrett et al., J Psychopharmacol 2016; 30(12):1279-95). Understanding the range and severity of symptoms is useful in tailoring patient care and anticipating patient needs during future treatment sessions. The challenging experience questionnaire (CEQ) addresses seven prominent factors (grief, fear, death, insanity, isolation, physical distress, and paranoia) and provides a phenomenological profile of challenging aspects that a patient has or may experience during treatment (Barrett et al., J Psychopharmacol 2016; 30(12):1279-95). There are 26 items included in the CEQ evaluation, and each response carries a numerical value, ranging from 0-5, wherein 0 is ‘none; not at all’ and 5 is ‘extreme (more than ever before in my life).’ Across these 26 items, the seven factors, or sub-categories, comprise different items, wherein grief consists of items 2, 6, 9, 11, 23, and 25; fear consists of items 4, 7, 14, 21, and 26; death consists of items 16 and 20; insanity consists of items 8, 13, and 19; isolation consists of items 1, 10, and 24; physical distress consists of items 3, 5, 15, 17, and 18; and paranoia consists of items 12 and 22. Responses are summed within each sub-category and converted to a percentage of the total possible points. Further, responses for all item are summed and converted to a percentage of the total possible points.

In some aspects, the embodiments provided herein include assessment of a change from baseline in the challenging experience questionnaire (CEQ) evaluation. In some aspects, the CEQ evaluation is completed at one or more time point, such as at 1 day post administration of one or more doses of psilocybin.

d. Monitor Rating Scale (MRS)

The monitor rating scale (MRS) questionnaire is completed by a clinician and involves rating and scoring dimensions of participant's behavior and/or mood during the treatment session (Griffiths et al., Psychopharmacology Berl 2006; 187(3):268-83). There are 20 items (or dimensions) included in the MRS evaluation, and some dimension are assigned a numerical value, ranging from 0-4, wherein 0 is ‘none; no effect’ and 4 is ‘peak effect; happening often.’ For other dimensions, scoring is assigned as the total duration (in minutes, across a defined window of time) observed for a patient behavior (e.g., talking with the clinician or total speech). Dimension scores are summed within each sub-category or across all dimensions.

In some aspects, the embodiments provided herein include assessment of a change from baseline in the monitor rating scale (MRS) questionnaire evaluation. In some aspects, the MRS evaluation is completed at one or more time point, such as at 1 day post administration of one or more doses of psilocybin.

In some embodiments, the MRS score for an individual receiving treatment improves during and/or after treatment, for example one or more MRS score improves by at least 1 points, at least 5 points, at least 10 points, at least 15 points, at least 20 points, or at least 30 points.

### B. Clinical Indicators

In some aspects, one or more indicators include clinical indicators, such as one or more indicators that can be measured in the clinic, for example, metabolic biomarkers or neurological indicators.

### 1. Metabolic Biomarkers

In some aspects, BED can result in obesity and weight-related health disorders (e.g., diabetes) due to excessive and poor eating habits. An individual may experience dysfunctional or uncontrollable hunger cravings and/or a lack of satiety after eating, leading to excessive consumption beyond current bodily energy needs (Heymsfield et al., Obesity (Silver Spring). 2014; 22(0 1): S1-S17). Hunger and satiety regulation, as well as glucose metabolism and regulation, can be assessed by quantifying important metabolites involved in these pathways.

In some aspects, the embodiments provided herein include assessment of a change from baseline in metabolic biomarkers assessment. In some aspects, an individual receiving treatment in accordance with the provided embodiments is subject to metabolic biomarkers measurements. In some aspects, exemplary measurements include plasma level quantification of key metabolic biomolecules including ghrelin, leptin, adiponectin, glucose, and insulin and/or a homeostasis model assessment of insulin resistance. In some embodiments the metabolic biomarker measurements are collected using one or more quantitative analytical techniques, such as mass spectrometry, liquid chromatography, antibody-based detection and quantification, immunoassay, activity-based assay, or other metabolite and analyte detection methods.

In some embodiments the plasma level quantification of key metabolic biomarkers, for example, one or more of ghrelin, leptin, adiponectin, glucose, and insulin and/or a homeostasis model assessment of insulin resistance, is evaluated or measured at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

a. Hunger and Satiety Regulation

Hunger is a strong desire or physical need for food, while satiety is the feeling of fullness that persists after eating (Smith and Ferguson, Dev Disabil Res Rev 2008; 14:96-104). These represent two important factors in the regulation of normal eating habits, wherein dysregulation may result in overconsumption and weight-related issues, including obesity (Benelam B., Nutr Bull 2009; 34:126-73). Hunger and satiety regulation are extremely complex and rely on multiple signaling pathways within and outside of the central nervous system (CNS) (Graaf et al., Am J Clin Nutr 2004; 79(6):946-61). This regulation comprises a temporal aspect, where a subset of biomolecules are involved in short-term regulation (e.g., ghrelin), while others effect long-term hunger regulation (e.g., leptin and adiponectin). As hallmark features of normal metabolism and deviations thereof, quantification of these metabolic biomolecules (or biomarkers) is an essential component of understanding the physiological status of an individual suffering from a metabolic disorder, including BED. In some embodiments, the change from baseline in one or more aspect of hunger and satiety regulation is utilized to evaluate the treatment of BED. In some embodiments, the aspect of hunger and satiety regulation includes, for example, the level of ghrelin, the level of leptin, and/or the level of adiponectin.

### (i) Ghrelin

Ghrelin is a short peptide hormone that functions as a key regulator of nutrient sensing, meal initiation, and appetite. Ghrelin is often referred to as the “hunger hormone” because elevated levels are associated with hunger and increased food consumption (Davis, J. Brain Res 2018; 1693(Pt B):154-158). With important regulatory roles across the body, ghrelin is a critical factor in the development of obesity, insulin resistance and diabetes (Pradhan et al., Curr Opin Clin Nutr Metab Care 2013; 16(6):619-24.). In some embodiments, the change from baseline in the level of ghrelin is utilized to evaluate the treatment of BED. In some embodiments the quantification of ghrelin is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration. In some embodiments, the level of ghrelin is reduced in a patient receiving treatment, for example the decrease in the level of ghrelin is at least 1%, at least 2%, at least 3%, at least 4%, at least 5%, or at least 10%, compared to baseline.

### (ii) Leptin

Leptin plays a critical role in the regulation of energy balance and metabolic homeostasis. Impairment of leptin signaling is closely involved in the pathogenesis of obesity and metabolic diseases (e.g., diabetes and cardiovascular disease) (Obradovic et al., Front Endocrinol (Lausanne) 2021; 12:585887). Leptin functions to suppress appetite, reduce food intake, and promote mitochondrial oxidation and thermogenesis (Liu et al., Adv Exp Med Biol 2018; 1090:123-144). In some embodiments, the change from baseline in the level of leptin is utilized to evaluate the treatment of BED. In some embodiments the quantification of leptin is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration. In some embodiments, the level of leptin is elevated in a patient receiving treatment, for example the increase in the level of leptin is at least 1%, at least 2%, at least 3%, at least 4%, at least 5%, or at least 10%, compared to baseline.

(iii) Adiponectin

Adiponectin (adipocyte complement-related protein of 30 kDa, or Acrp30) is an adipokine endocrine factor, synthesized and released from adipose tissue. Adiponectin is the most abundant peptide secreted by adipocytes, whose reduction plays a central role in obesity-related diseases, including type 2 diabetes and cardiovascular disease (Achari and Jain, Int J Mol Sci 2017; 18(6):1321). In some embodiments, the change from baseline in the level of adiponectin is utilized to evaluate the treatment of BED. In some embodiments the quantification of adiponectin is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration. In some embodiments, the level of adiponectin is elevated in a patient receiving treatment, for example the increase in the level of adiponectin is at least 1%, at least 2%, at least 3%, at least 4%, at least 5%, or at least 10%, compared to baseline.

b. Glucose Metabolism and Regulation

Improper glucose metabolism and regulation is a hallmark feature of disease that is most often associated with type 2 diabetes (Roden and Shulman, Nature 2019; 576(7785):51-60). Aberrant glucose regulation and/or insulin sensitivity have known genetically origins, but are heavily influenced by environmental and lifestyle factors as well. Understanding the status of glucose metabolism and regulation in an individual having BED is essential for a comprehensive evaluation. In some embodiments, the change from baseline in glucose metabolism and regulation is utilized to evaluate the treatment of BED. In some embodiments the quantification of glucose is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

### (i) Glucose

In some embodiments, the level of glucose is utilized to evaluate the treatment of BED. For an average adult, blood glucose is defined as normal blood glucose when fasting glucose is less than 100 mg/dL before a meal and is less than 140 mg/dL two hours after eating; as elevated blood glucose when fasting glucose is between 100-125 mg/dL before a meal and is between 140-199 mg/dL two hours after eating; as high blood glucose when fasting glucose is above 126 mg/dL before a meal and is more than 200 mg/dL two hours after eating.

In some embodiments the quantification of glucose is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration. In some embodiments, the level of glucose is reduced in a patient receiving treatment, for example the decrease in the level of glucose is at least 1%, at least 2%, at least 3%, at least 4%, at least 5%, or at least 10%, compared to baseline.

### (ii) Insulin

Insulin is a hormone secreted by the β-cells of the pancreas (pancreatic islets of Langerhans) in response to food consumption and energy abundance, signaling for increased glucose uptake and storage to reduce and/or maintain blood glucose levels. Insulin orchestrates complex metabolic changes associated with in shifts in metabolic pathways, energy management, immune function, and other important aspects critical to healthy living. Lifestyle, environmental, and genetic factors can lead to insulin resistance, namely overeating, unhealthy eating habits, and a lack of physical activity or generally sedentary lifestyle. Insulin resistance is a major risk factor for the development of one or more disease states including but not limited to diabetes, metabolic syndrome, heart disease, liver disease, polycystic ovary syndrome, cancer, and cognitive decline (PCOS) (Wilcox G., Clin Biochem Rev 2005; 26(2):19-39). For an average adult, a level of insulin is defined as the normal insulin when insulin is less than 25 μU/L during fasting, 30-230 μU/L 30 minutes after glucose administration, 18-276 μU/L 1 hour after glucose administration, and 16-166 μU/L 2 hour after glucose administration.

In some embodiments the quantification of insulin is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

(iii) Insulin Resistance

Insulin resistance is a term denoting some degree of dysfunction and/or functional impairment regarding the effects of insulin on glucose uptake, storage, and metabolism (Kahn and Flier J Clin Ivest 2000; 106(4):473-81; Wilcox G., Clin Biochem Rev 2005; 26(2):19-39). Due to the essential role that insulin and proper insulin sensitivity play in maintaining proper health, insulin resistance is a major risk factor for the development of one or more disease states including but not limited to diabetes, metabolic syndrome, heart disease, liver disease, polycystic ovary syndrome, cancer, and cognitive decline (PCOS). The homeostatic model assessment of insulin resistance (HOMA-IR) is a common analysis strategy for assessing the spectrum of insulin sensitivity and resistance, wherein the HOMA-IR is defined by a formula of fasting plasma glucose (mmol/L) times fasting serum insulin (mU/L) divided by 22.5 (Matthews et al., Diabetologia 1985; 28:412-19). For an average adult, insulin sensitivity is define as normal or high insulin sensitivity by a HOMA-IR score below 1.0; as insulin resistance by a HOMA-IR score above 1.9; and as significant insulin resistance by a HOMA-IR score below 2.9. In some embodiments insulin resistance is determine in using a HOMA-IR approach. In some embodiments, the HOMA-IR is completed at one or more time points, for example, prior to administration of one or more doses of psilocybin, and at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration. In some embodiments, the HOMA-IR score is reduced in a subject receiving treatment, for example the decrease in the HOMA-IR score is decreased at least 0.1 point, at least 0.2 point, at least 0.3 point, at least 0.4 point, at least 0.5 point, at least 1 point, at least 1.5 points, or more than 2 points compared to baseline.

### 2. Neurophysiologic Clinical Indicators

Neurophysiologic clinical indicators, such as brain morphology, connectivity, and functionality, can be measured using various methodologies, technical advances, and the rapidly evolving field of neuroimformatics, a key factor in the subsequent analysis of large datasets from population size cohorts (Roalf and Gur, Neuropsychology 2017; 31(8):954-71; Woo et al., Nat Neurosci 2017; 20(3):365-77; Horien et al., Nat Hum Behav 2021; 5(2):185-93). These indicators can be used to measure brain-specific changes more quantitatively during development, aging, learning, disease, and in response to acute stimuli, including small-molecule compounds and/or biomolecules (Borsook et al., Nat Rev Drug Discov 2006; 5(5):411-24; Matthews and Hampshire, Neuron 2016; 91(3):511-28; Carmichael et al., Drug Discov Today 2018; 23(2):333-48). Such indicators can be used to measure changes and improvements during and after administration of psychedelic compounds such as psilocybin, in accordance with the provided embodiments.

In some aspects, an individual receiving treatment in accordance with the provided embodiments is subject to one or more neurophysiologic clinical indicator measurement, such as voxel-based morphometry, functional magnetic resonance imaging (fMRI)-based multistate functional connectivity and activation analyses, and/or electroencephalogram (EEG) analysis. In some cases, an individual receiving treatment in accordance with the provided embodiments is subject to one or more neurophysiologic clinical indicator measurement, for example to quantify brain morphology, including grey matter volume and structure, and brain activity, including resting EEG activity and multistate functional connectivity and activation patterns.

a. Brain Morphology

In some aspects, brain morphology, including grey matter volume and structure, can be assessed (Dolz et al., Comput Med Imaging Graph 2020; 79:101660; Shofty et al., World Neurosurg 2020; 134:e1143-e1147). Grey matter, the area comprising most of brain's neuronal cell bodies (Lee et al., Sci Total Environ 2020; 707:135603; Chiao et al., Methods Mol Biol 2020; 2092:65-75), is associated with proper cognitive performance, wherein decreased grey matter volume corresponds to cognitive impairment (Spulber et al., Curr Alzheimer Res 2012; 9(4):516-24; Valdes et al., Neuroimage Clin 2020; 25:102158; Dicks et al., Neuroimage Clin 2019; 22:101786). Voxel-based morphometry (VBM) is a neuroimaging technique that quantifies focal differences in brain anatomy, wherein images of core components within the brain (e.g., grey matter, white matter, and cerebrospinal fluid) are anatomically normalized and processed according to various computational algorithms and statistics (Wright et al., Schizophr Res 1999; 35(1):1-14; Ashbumer and Friston Neuroimage 2000; 11(6 Pt 1):805-21). VBM thus allows global and regional volumetric comparisons between different groups (Andujar et al., Brain Sci 2021; 11(8):999).

In some embodiments, the change from baseline in VBM is utilized to evaluate the brain morphology of an individual receiving treatment. In some embodiments, a resting VBM is performed prior to and after treatment. In some embodiments, a resting VBM is performed about 1 week prior to treatment initiation and about 4 weeks and/or 8 weeks, such as about 4 weeks, after administration of the one or more doses of psilocybin. In some embodiments, the gray matter volume for an individual receiving treatment increases during and/or after treatment, for example the gray matter volume increases by at least 1%, at least 2%, at least 3%, at least 4%, at least 5%, or at least 10%, compared to baseline.

b. Brain Activity

Brain activity can be routinely monitored using non-invasive methods such as an EEG and an fMRI (Abreu et al., Front Hum Neurosci 2018; 12:29). An EEG records electrical activity via the scalp on the millisecond timescale, wherein the recorded signal represents the macroscopic activity of the brain. An fMRI detects changes in blood flow within the brain, wherein these changes correspond to regional and site-specific changes in activity. In some embodiments, a brain activity measurements are performed about 1 week prior to treatment initiation and about 4 weeks and/or 8 weeks, such as about 4 weeks, after administration of the one or more doses of psilocybin. In some embodiments, brain activity is utilized to evaluate the treatment of BED, for example with an fMRI and/or an EEG analysis.

### (i) Functional Activation and or Connectivity

In some aspects, functional connectivity and activation are often characterized with a functional magnetic resonance imaging (fMRI) analysis, where arterial spin labelling (ASL) and blood oxygen level dependent (BOLD) are used to measure changes in blood flow and functional connectivity of different brain regions at various times (e.g., before, during, and/or after treatment) (Soares et al., Front Neurosci 2016; 10:515). In some embodiments, the change from baseline in the functional connectivity and/or activation is utilized to evaluate the treatment of BED.

In some aspects, exemplary indicators measured with fMRI include functional connectivity (resting state; fed and fasted), functional activation (food cue reactivity task; fed and fasted), functional connectivity (food cue reactivity task; fed and fasted), and/or voxel Based morphometry (grey matter volume/structure).

In some embodiments, the functional connectivity evaluation is completed in the resting state, wherein the individual receiving treatment is either fed or fasted. In some embodiments, the functional connectivity and/or activation evaluation is completed in the task-related state, wherein the individual receiving treatment is present with a food cue reactivity tasked and is either fed or fasted. In some embodiments, the resting state function connectivity fMRI is performed prior to and after treatment. In some embodiments, the task-related function activation and connectivity fMRI is performed prior to and after treatment. In some embodiments, the resting state function connectivity fMRI is performed about 1 week prior to treatment initiation and about 4 weeks and/or 8 weeks, such as about 4 weeks, after administration of one or more doses of psilocybin. In some embodiments, the task-related function activation and connectivity fMRI is performed about 1 week prior to treatment initiation and about 4 weeks and/or 8 weeks, such as about 4 weeks, after administration of one or more doses of psilocybin.

In some aspects, the embodiments provided herein include assessment of a change from baseline in functional magnetic resonance imaging (fMRI) analysis. In some aspects, the fMRI is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the fMRI data for an individual receiving treatment changes during and/or after treatment.

### (ii) Electroencephalogram (EEG)

Despite having poor spatial sensitivity, the EEG is both convenient and effective for recording high-resolution temporal brain activity (Michel and Brunet, Front Neurol 2019; 10:325). Direct electrical measurements on the scalp provide millisecond timescale information, compared seconds and minutes needed for other preferred methodologies. The EEG is limited to measuring the uppermost layer of the brain, but when paired with other brain activity measurements (e.g., an fMRI), the EEG provides key data for generating a comprehensive view and quantitative assessment of brain activity (Abreu et al., Front Hum Neurosci 2018; 12:29). In some embodiments, the change from baseline in the EEG is utilized to evaluate the treatment of BED.

In some aspects, exemplary indicators measured with EEG include explosive synchronization of network activity at rest and Power spectrum analysis (frequency bands).

In some embodiments, a resting EEG is performed prior to and after treatment. In some embodiments, a resting EEG is performed about 1 week prior to treatment initiation and about 4 weeks and/or 8 weeks, such as about 4 weeks, after administration of one or more doses of psilocybin. In some aspects, the embodiments provided herein include assessing a change from baseline in electroencephalogram (EEG) analysis. In some aspects, the EEG is completed at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration, such as at or about 4 weeks after administration.

In some embodiments, the resting state EEG data for an individual receiving treatment changes during and/or after treatment.

### C. Body- and Weight-Related Indicators and Laboratory Parameters

In some aspects, one or more indicators include body- and weight-related indicators and various laboratory parameters, such as vital signs and hematology and blood chemistry parameters. Body- and weight-related indicators are convenient and classic metrics for estimating a subject health and medical risks, including but not limited to body mass index (BMI) and waist circumference (USPSTF Ann Intern Med 2003; 139(11):930-2; Barton M., Pediatrics 2010; 125(2):361-7). In some embodiments, the body and/or weight indicator is a change from baseline in BMI, waist circumference, and/or body weight.

### 1. Body Mass Index (BMI)

Body mass index (BMI) is a person's weight in kilograms divided by the square of height in meters, and it represents a convenient screening method for individuals having high levels of fatness, wherein that individual is likely overweight or nearly overweight (Gallegher et al., Am J Epidemiol 1996; 143(3):228-39). In extreme cases, the individual may be obese or morbidly obese, wherein weight-related health complications can be serious and life-threatening. Individuals with high BMI levels are at increased risk for diseases and adverse health conditions including but not limited to mortality (all causes of death), hypertension, elevated cholesterol, elevated triglycerides, diabetes, coronary heart disease, stroke, gallbladder disease, osteoarthritis, sleep apnea and other breathing complications, chronic inflammation, certain types of cancer, decreased quality of life, depression, anxiety, increased pain, and decreased physical functionality (NHLBI 2013 Managing Overweight and Obesity in Adults: Systematic Evidence Review from the Obesity Expert Panel; Bhaskaran et al., Lancet 2014; 384(9945):755-65). For adults, a BMI between 18.5 and 24.9 is considered a healthy weight; below 18.5 is underweight; between 25.0 and 29.9 is overweight; and above 30.0 is obesity. Although BMI does not account for individuals having an athletic build or high amounts of lean muscle mass, athletes make up a small minority of the population. For most individuals, BMI and other weight-related indicators are quite accurate (Garrow and Webster Int J Obes 1985; 9(2):147-53; Freedman et al., Am J Clin Nutr 2013; 98(6):1417-24; Wohlfahrt-Veje et al., Eur J Clin Nutr 2014; 68(6):664-70; Flegal and Graubard Am J Clin Nutr 2009; 89(4):1213-19), and can function even better when used together (e.g., BMI and waist circumference) (Zhang et al., Circulation 2008; 117(13):1658-67; Ross et al., Nat Rev Endocrinol 2020; 16(3):177-89). In some embodiments, the change from baseline in BMI is utilized to evaluate the treatment of BED.

In some aspects, the embodiments provided herein include an assessment of the maximum change from baseline in BMI after treatment. In some aspects, the assessment includes one or more BMI evaluations at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the BMI for an individual receiving treatment decreases during and/or after treatment, for example the BMI decreases by at least 1, at least 2, at least 3, at least 4, at least 5, or at least 10 compared to baseline.

### 2. Waist Circumference

Waist circumference a physical feature that is used to determine abdominal adiposity and general obesity (Ross et al., Nat Rev Endocrinol 2020; 16(3):177-89). Waist circumference is well-correlated with BMI, and similarly, larger waist sizes are associated with weight-related health complications that can be serious and life-threatening. For adult men, an individual with a waist circumference above 40 inches is considered obese. For adult women, an individual with a waist circumference above 35 inches is considered obese. In some embodiments, the change from baseline in waist circumference is utilized to evaluate the treatment of BED.

In some aspects, the embodiments provided herein include an assessment of maximum change from baseline in waist circumference after treatment. In some aspects, the assessment includes one or more waist circumference evaluations at one or more of the time points following administration of one or more doses of psilocybin, such as between at or about 1 months to 36 months after administration of one or more doses of psilocybin, such as at or about 1 month, 2 months, 3 months, 4 months, 5 months, 6 months, 12 months, 18 months, 24 months, 30 months or 36 months, after the administration.

In some embodiments, the waist circumference for an individual receiving treatment decreases during and/or after treatment, for example the waist circumference decreases by at least at or about 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, 40 cm, 45 cm, or 50 cm compared to baseline.

### 3. Weight

Increased body weight, along with measures of BMI and waist circumference, is strongly correlated with negative health consequences and outcomes as described in the sections above. In extreme cases, the individual may be obese or morbidly obese, wherein weight-related health complications can be serious and life-threatening. Individuals with elevated body weight are at increased risk for diseases and adverse health conditions including but not limited to mortality (all causes of death), hypertension, elevated cholesterol, elevated triglycerides, diabetes, coronary heart disease, stroke, gallbladder disease, osteoarthritis, sleep apnea and other breathing complications, chronic inflammation, certain types of cancer, decreased quality of life, depression, anxiety, increased pain, and decreased physical functionality (NHLBI 2013 Managing Overweight and Obesity in Adults: Systematic Evidence Review from the Obesity Expert Panel; Bhaskaran et al., Lancet 2014; 384(9945):755-65). In some cases, for adults of an average height, a body weight between 125 and 174 pounds; between 175 and 204 pounds is overweight; and more than 204 pounds is considered obese. In some embodiments, the change from baseline in body weight is utilized to evaluate the treatment of BED.

In some aspects, the embodiments provided herein include an assessment of the maximum change from baseline in weight after treatment. In some aspects, the assessment includes one or more weight evaluations at one or more of the time points following administration of one or more doses of psilocybin, such as between at or about 2 weeks and 20 weeks after administration of one or more doses of psilocybin, such as at or about 2 weeks, 4 weeks, 6 weeks, 8 weeks, 10 weeks, 12 weeks, 14 weeks and/or 16 weeks after the administration.

In some embodiments, the body weight for an individual receiving treatment decreases during and/or after treatment, for example the body weight decreases by at least at or about 1 kg, 2 kg, 3 kg, 4 kg, 5 kg, 6 kg, 7 kg, 8 kg, 9 kg, 10 kg, 12.5 kg, 15 kg, 17.5 kg, or 20 kg, compared to baseline.

### D. Exemplary Monitoring and Evaluation

An exemplary schedule of evaluations comprising physical measures of treatment outcomes, observer-rated and subject-reported outcomes, quantification of clinical indicators, and combinations thereof described in the methods of this invention are summarized in Table 3.

TABLE 3
Exemplary Monitoring and Evaluation Scheme

| | | | | Prep | | Integration | Integration | Therapy | Safety | Follow- |
|---|---|---|---|---|---|---|---|---|---|---|
| Event/ | | | | Sessions | | Session 1 1 | Session | Follow- | Follow- | up/Early |
| Procedure | Screening | Run-in | | 1 & 2¹ | Medication | Day | 2¹ | up 1 | up | Discharge |
| Timing | Weeks −5 | Weeks | Baseline | Week 1 | Dosing | after | Week 3 | Week 4 | Weeks | Weeks |
| Windows of | and −4 | −3 | Week −1 | Video/ | Day 1 | dose | Video/ | Video/ | 4, 5, 8, | 6, 10, 14 |
| ±2 days | In- | and −2 | In- | In- | Week 2 | In- | In- | In- | 12 | In- |
| Visit type | person | Remote | person | person | In Unit | person | person | person | Telephone | person |
| Informed | | | | | | | | | | |
| Consent | | | | | | | | | | |
| Psychiatric | X | | | | | | | | | |
| History ² | | | | | | | | | | |
| Medication | X | | | | | | | | | |
| History | | | | | | | | | | |
| Entry Criteria | X | | | | | | | | | |
| Vital Signs ³ | X | | | | X | X | | | | X |
| 12-lead ECG ⁴ | | | | | X | X | | | | X |
| Physical Exam | X | | | | | X | | | | X |
| Height, | X | | | | X | | | | | X |
| Weight, BMI | | | | | | | | | | |
| Waist | X | | | | X | | | | | X |
| circumference | | | | | | | | | | |
| Chemistry ⁵ | X | | | | X | X | | | | X |
| hematology ⁶ | | | | | | | | | | |
| Urinalysis ⁷, | X | | | | X ⁹ | | | | | |
| drug screen ⁸ | | | | | | | | | | |
| Alcohol breath | X | | | | X ⁹ | | | | | |
| test | | | | | | | | | | |
| Pregnancy test | X | | | | X | | | | | |
| ¹⁰ | | | | | | | | | | |
| Therapy | | | | X | | X | X | X | | |
| session ¹¹ | | | | | | | | | | |
| fMRI and EEG | | | X | | | | | | | X ¹² |
| Administer | | | | | X | | | | | |
| oral psilocybin | | | | | | | | | | |
| C-SSRS ¹³ | X | | X | | X | X | X | X | X | X |
| MEQ30 | | | | | | X | | | | |
| CEQ | | | | | | X | | | | |
| EBI | | | | | | X | | | | |
| MRS | | | | | X ¹⁴ | | | | | |
| Metabolic | | | | | X | | | | | X |
| biomarkers | | | | | | | | | | |
| BES | X | | | | | | | | | X |
| Binge eating | | X | X | X | | X | X | | X ¹⁵ | X ¹⁵ |
| (modified | | | | | | | | | | |
| Eating | | | | | | | | | | |
| Questionnaire, | | | | | | | | | | |
| daily) | | | | | | | | | | |
| Binge eating | X ¹⁶ | | | | | | | | | X ¹⁷ |
| (Eating | | | | | | | | | | |
| Questionnaire, | | | | | | | | | | |
| q 4 wks) | | | | | | | | | | |
| CGI-I | X | | | | | | | | | X |
| PGI-I | X | | | | | | | X | | X |
| HADS ¹³ | X | | | | | | | X | | X |
| AAQ-II | X | | | | | | | X | | X |
| Record | X ¹⁸ | X | X | X | X ¹⁹ | X | X | | X | X |
| adverse events | | | | | | | | | | |
| Concomitant | X | | X | X | X | X | X | | X | X |
| medications | | | | | | | | | | |

¹ Described herein.
² Psychiatric history assessed by PI and study psychologist.
³ Full seated vital signs (heart rate and blood pressure) at screening and upon admission to clinic on dosing day. Brief vital signs will be assessed before capsule administration and at 30, 60, 90, 120, 180, 240, 300, and 360 minutes after capsule administration and the following day. Brief seated vital signs at each in-person follow up visit.
⁴ 12-lead ECG in triplicate at screening, pre-dose and 60, 120, and 360 minutes after capsule administration. ECGs will also be obtained for early terminators.
⁵ Chemistry panel (Na⁺, K⁺, Cl⁻, HCO3⁻ , Ca⁺⁺, Mg⁺⁺, P, BUN, creatinine, glucose, total bilirubin, albumin, ALT, AST, GGT, CK, LDH, alkaline phosphatase) on dosing day prior to dosing.
⁶ CBC with white cell differential and platelet count on dosing day prior to dosing.
⁷ Urinalysis includes pH, specific gravity, protein, occult blood, glucose, ketones; plus microscopic examination of sediment for RBC, WBC, epithelial cells, casts, crystals and bacteria.
⁸ Urine drug screen to be standard 14 (AMP, BUP, BZO, COC, mAMP, MDMA, MOP, MTD, OXY, THC, ETG, FTY, TRA, K2).
⁹ Urine drug screen and alcohol breath test performed prior to dosing on dosing day.
¹⁰ Pregnancy test for women of reproductive potential. Test performed prior to dosing on dosing day.
¹¹ Further details on therapy sessions are described herein.
¹² fMRI and EEG done at Week 6 visit only.
¹³ On dosing day, complete prior to dosing and prior to discharge. During follow up, if there are psychiatric concerns, C-SSRS can be done weekly along with HADS.
¹⁴ Conducted at same time as vital signs during dosing session.
¹⁵ Completed daily through Weeks 4, 5, and 6 only.
¹⁶ Completed once for the 4 weeks prior to Screening
¹⁷ Completed once at Weeks 10 and 14 only.
¹⁸ Medical occurrences that begin before the Baseline visit but after obtaining informed consent will be recorded as medical history, not as adverse events.
¹⁹ On dosing day, complete prior to dosing and prior to discharge.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

In some aspects, the term “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

In some aspects, integers, steps, or elements of the technology recited herein as singular integers, steps or elements clearly encompass both singular and plural forms of the recited integers, steps or elements.

In some contexts, the terms ‘a’ and ‘an’ are used to refer to one or more than one (i.e., at least one) of the grammatical object of the article. By way of example, reference to ‘an element’ means one element, or more than one element.

In some contexts, the term ‘about’ means that reference to a figure or value is not to be taken as an absolute figure or value, but includes margins of variation above or below the figure or value in line with what a skilled person would understand according to the art, including within typical margins of error or instrument limitation. In some cases words, ‘about’ is understood to refer to a range or approximation that a person or skilled in the art would consider to be equivalent to a recited value in the context of achieving the same function or result.

In some contexts, the terms “treating”, “treatment” and “therapy” refer to curative therapy, prophylactic therapy, palliative therapy and preventative therapy. Thus, in the context of the present disclosure the term “treating” encompasses curing, ameliorating, or tempering the severity of a psychological condition or one or more of its associated symptoms. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. The terms do not imply complete curing of a disease or complete elimination of any symptom or effect(s) on all symptoms or outcomes.

In some contexts, the terms “therapeutically effective amount” or “pharmacologically effective amount” or “effective amount” refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimizing adverse side effects, including side effects typically associated with other therapies.

In some contexts, a “pharmaceutical carrier,” “diluent” or “excipient” includes, but is not limited to, any physiological buffered (i.e., about pH 7.0 to 7.4) medium comprising a suitable water soluble organic carrier, conventional solvents, dispersion media, fillers, carriers, coatings, antibacterial and antifungal agents, isotonic and absorption delaying agents.

“Subject” includes any human.

In some contexts, the term “administering” and variations of that term including “administer” and “administration”, includes contacting, applying, delivering, or providing a compound or composition of the invention to a subject by any appropriate means.

### IV. EXEMPLARY EMBODIMENTS

Among the provided embodiments are:
1. A method of treating binge eating disorder (BED), the method comprising:
orally administering to a subject suffering from one or more symptoms of BED, one or more doses of psilocybin or an active metabolite thereof effective to induce a dissociative state;
providing one or more integration sessions to the subject after emergence from the dissociative state; and
assessing in the subject one or more indicators prior to and/or after administration of the one or more doses of psilocybin or an active metabolite thereof; wherein the one or more indicators are associated with the one or more symptoms of BED, dissociative state, treatment outcome, safety and/or is related to the subject.
2. A method of treating binge eating disorder (BED), the method comprising assessing in the subject one or more indicators prior to and/or after administration of the one or more doses of psilocybin or an active metabolite thereof in a subject suffering from one or more symptoms of BED that has been provided one or more integration sessions after emergence from a dissociative state induced by orally administered one or more doses of psilocybin or an active metabolite thereof effective to induce a dissociative state; wherein one or more indicators associated with the one or more symptoms of BED, dissociative state, treatment outcome, safety and/or is related to the subject are assessed in the subject prior to and/or after administration of the one or more doses of psilocybin or an active metabolite thereof.
3. The method of embodiment 1 or 2, wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).
4. A method of treating binge eating disorder (BED), the method comprising:
orally administering to a subject suffering from one or more symptoms of BED, one or more doses of psilocybin or an active metabolite thereof effective to induce a dissociative state;
providing one or more integration sessions to the subject after emergence from the dissociative state; and
assessing in the subject one or more indicators prior to and/or after administration of the one or more doses of psilocybin or an active metabolite thereof; wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).
5. A method of treating binge eating disorder (BED), the method comprising assessing in the subject one or more indicators prior to and/or after administration of the one or more doses of psilocybin or an active metabolite thereof in a subject suffering from one or more symptoms of BED that has been provided one or more integration sessions after emergence from a dissociative state induced by orally administered one or more doses of psilocybin or an active metabolite thereof effective to induce a dissociative state; wherein one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II) are assessed in the subject prior to and/or after administration of the one or more doses of psilocybin or an active metabolite thereof.
6. A method of treating binge eating disorder (BED), the method comprising:
orally administering to a subject suffering from one or more symptoms of BED, one or more doses of psilocybin or an active metabolite thereof effective to induce a dissociative state; and
providing one or more integration sessions to the subject after emergence from the dissociative state.
7. A method of treating binge eating disorder (BED), the method comprising providing one or more integration sessions a subject suffering from one or more symptoms of BED after emergence from a dissociative state induced by orally administered one or more doses of psilocybin or an active metabolite thereof effective to induce a dissociative state.
8. The method of embodiment 6 or 7, further comprising assessing in the subject one or more indicators prior to and/or after administration of the one or more doses of psilocybin or an active metabolite thereof.
9. The method of embodiment 8, wherein the one or more indicators are associated with the one or more symptoms of BED, dissociative state, treatment outcome, safety and/or is related to the subject.
10. The method of any of embodiments 1-9, further comprising, prior to administering the one or more doses of psilocybin or an active metabolite thereof, assessing one or more of the following parameters in the subject:
   (1) risk of suicide;
   (2) vital signs;
   (3) incidence of cardiovascular conditions;
   (4) incidence of gastrointestinal disease;
   (5) incidence of epilepsy;
   (6) incidence of schizophrenia spectrum or other psychotic disorders;
   (7) family history of psychosis;
   (8) a moderate or severe alcohol or drug use disorder;
   (9) prior adverse effects from psilocybin or an active metabolite thereof; and
   (10) use of prior or concomitant medications.
11. The method of embodiment 10, further comprising identifying the subject for treatment with psilocybin or an active metabolite thereof if the subject meets the criteria for the one or more parameters.
12. A method of identifying a subject for treating binge eating disorder (BED) with psilocybin or an active metabolite thereof, the method comprising:
(a) assessing in a subject suffering from one or more symptoms of BED, one or more of the following parameters:
   (1) risk of suicide;
   (2) vital signs;
   (3) cardiovascular conditions;
   (4) presence of gastrointestinal disease;
   (5) incidence of epilepsy;
   (6) incidence of schizophrenia spectrum or other psychotic disorders;
   (7) family history of psychosis;
   (8) a moderate or severe alcohol or drug use disorder;
   (9) prior adverse effects from psilocybin or an active metabolite thereof; and
   (10) use of prior or concomitant medications;
(b) identifying the subject for treatment with one or more doses of psilocybin or an active metabolite thereof if the subject meets a criteria for the one or more parameters.
13. A method of identifying a subject for treating binge eating disorder (BED) with one or more doses of psilocybin or an active metabolite thereof, the method comprising identifying a subject suffering from one or more symptoms of BED for treatment with psilocybin or an active metabolite thereof if the subject meets a criteria for one or more of the following parameters that are assessed in the subject:
   (1) risk of suicide;
   (2) vital signs;
   (3) cardiovascular conditions;
   (4) presence of gastrointestinal disease;
   (5) incidence of epilepsy;
   (6) incidence of schizophrenia spectrum or other psychotic disorders;
   (7) family history of psychosis;
   (8) a moderate or severe alcohol or drug use disorder;
   (9) prior adverse effects from psilocybin or an active metabolite thereof; and
   (10) use of prior or concomitant medications.
14. The method of embodiment 12 or 13, further comprising:
orally administering to the identified subject, one or more doses of psilocybin or an active metabolite thereof effective to induce a dissociative state; and
providing one or more integration sessions to the subject after emergence from the dissociative state.
15 The method of any of embodiments 6-14, further comprising assessing in the subject one or more indicators prior to and/or after administration of the one or more doses of psilocybin or an active metabolite thereof.
16. The method of embodiment 15, wherein the one or more indicators are associated with the one or more symptoms of BED, dissociative state, treatment outcome, safety and/or is related to the subject.
17. The method of any of embodiments 1, 2, 8-11, 15, and 16, wherein the one or more indicators comprise observer-rated parameters, subject-reported parameters and/or clinical indicators.
18. The method of embodiment 1-17, wherein the one or more indicators are assessed prior to the administration as a baseline measurement, and after the administration as an outcome measurement.
19. The method of embodiment 18, wherein the changes between the baseline measurement and the outcome measurement are determined.
20. The method of embodiment 19, wherein the subject exhibits amelioration of the one or more indicators between the baseline measurement and the outcome measurement.
21. The method of any of embodiments 1, 2, 8-11, and 15-20, wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).
22. The method of any of embodiments 1-5, 8-11, and 15-21, wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, and body mass index (BMI).
23. The method of any of embodiments 1-5, 8-11, and 15-22, wherein the one or more indicators comprises frequency of binge eating episodes.
24. The method of any of embodiments 1-5, 8-11, and 15-23, wherein the frequency of binge eating episodes is measured using the Eating Questionnaire.
25. The method of any of embodiments 1-5, 8-11, and 15-24, wherein the frequency of binge eating episodes is measured daily.
26. The method of any of embodiments 1-5, 8-11, and 15-22, wherein the one or more indicators comprises the number of binge eating episodes per day.
27. The method of any of embodiments 1-5, 8-11, and 15-22, wherein the one or more indicators comprises interest in eating trigger foods.
28. The method of any of embodiments 1-5, 8-11, and 15-22, wherein the one or more indicators are selected from among Emotional Breakthrough Inventory (EBI), Acceptance and Action Questionnaire-II (AAQ-II), Patient Global Impression-Improvement (PGI-I), and Hospital Anxiety and Depression Scale (HADS).
29. The method of any of embodiments 1-5, 8-11, and 15-22, wherein the one or more indicators are one or more neurophysiologic biomarkers selected from among Resting state functional connectivity (fed and fasted) by functional magnetic resonance imaging (fMRI), task-related functional activation and connectivity during a food cue reactivity task (fed and fasted) by fMRI, voxel-based morphometry (grey matter volume/structure) by fMRI, and resting electroencephalogram (EEG).
30. The method of any of embodiments 1-5, 8-11, and 15-22, wherein the one or more indicators are one or more metabolic biomarkers selected from among ghrelin, leptin, adiponectin, insulin, glucose, and HOMA-IR (Homeostatic Model Assessment of Insulin Resistance).
31. The method of any of embodiments 1-5, 8-11, and 15-22, wherein the one or more indicators selected from among Monitor Rating Scale (MRS), Mystical Experience Questionnaire (MEQ30), and Challenging Experiences Questionnaire (CEQ).
32. The method of embodiment 1-5, 8-11, and 15-31, wherein the one or more indicators are measured during one or more of the integration sessions.
33. The method of any of embodiments 1-5, 8-11, and 15-22, wherein the one or more indicators selected from among vital signs, blood chemistry and hematology tests, urinalysis, electrocardiograms (ECG), and Columbia-Suicide Severity Rating Scale (C-SSRS).
34. The method of embodiment 33, wherein the blood chemistry and hematology tests comprise measuring the levels of one or more of Na⁺, K⁺, Cl⁻, HCO₃⁻, Ca⁺⁺, Mg⁺⁺, P, BUN, creatinine, glucose, total bilirubin, albumin, ALT, AST, GGT, CK, LDH, alkaline phosphatase, complete blood count, white cell differential count and platelet count.
35. The method of embodiment 33, wherein the urinalysis comprises measuring one or more of pH, specific gravity, protein, occult blood, glucose, and ketones plus microscopic examination of sediment for RBC, WBC, epithelial cells, casts, crystals, and bacteria.
36. The method of any of embodiments 11-35, wherein the parameter is presence of gastrointestinal disease and the subject meets the criteria for the parameter if the subject does not have a gastrointestinal disease that could interfere with absorption of orally-administered psilocybin or an active metabolite thereof.
37. The method of any of embodiments 11-36, wherein the parameter is incidence of epilepsy and the subject meets the criteria for the parameter if the subject does not have epilepsy.
38. The method of any of embodiments 11-37, wherein the parameter is incidence of schizophrenia spectrum or other psychotic disorders and the subject meets the criteria for the parameter if the subject does not have schizophrenia spectrum or other psychotic disorder that meets the DSM-5 criteria, major depressive disorder with psychotic features, or Bipolar I or Bipolar II Disorder.
39. The method of any of embodiments 11-38, wherein the parameter is family history of psychosis, and the subject meets the criteria for the parameter if the subject does not have a family history of psychosis.
40. The method of any of embodiments 11-39, wherein the parameter is a moderate or severe alcohol or drug use disorder, and the subject meets the criteria for the parameter if the subject does not have a moderate or severe alcohol or drug use disorder that meets the DSM-5 criteria.
41. The method of any of embodiments 11-40, wherein the parameter is prior adverse effects from psilocybin or an active metabolite thereof, and the subject meets the criteria for the parameter if the subject does not have a prior adverse effects from psilocybin or an active metabolite thereof.
42. The method of any of embodiments 11-41, wherein the parameter is use of prior or concomitant medications, and the subject meets the criteria for the parameter if the subject is not using any of the following prior or concomitant medications: UGT1A9 inhibitors, 1A10 inhibitors, regorafenib, rifampicin, phenytoin, eltrombopag, mefenamic acid, diflunisal, niflumic acid, sorafenib, isavuconazole, deferasirox, Ginseng, aldehyde or alcohol dehydrogenase inhibitors, disulfiram, amphetamines, buprenorphine, benzodiazepines, cocaine, methamphetamines, Ecstasy (MDMA), morphine, methadone, oxycodone, marijuana, ethyl glucuronide, fentanyl, tramadol, synthetic cannabinoids (K2) regular use of psychoactive prescription medication, opioids, tramadol, or benzodiazepines; concomitant use of antidepressants; regular use of medications having a primary centrally-acting serotonergic effect, monoamine oxidase inhibitors (MAOIs), or selective serotonin reuptake inhibitors (SSRIs), or use of serotonin-acting dietary supplements, 5-hydroxy-tryptophan, St. John's wort,
43. The method of any of embodiments 11-42, wherein the subject is identified for treatment with psilocybin or an active metabolite thereof if the subject meets the criteria for all of the parameters (1)-(10).
44. The method of any one of embodiments 1-5, 8-11, and 15-43, wherein the one or more indicators are measured at about 4 weeks and at about 12 weeks following administration of the one or more doses of psilocybin or an active metabolite thereof.
45. The method of any one of embodiments 1-5, 8-11, and 15-44, wherein the one or more indicators are measured at about 4 weeks, at about 8 weeks and at about 12 weeks following administration of the one or more doses of psilocybin or an active metabolite thereof.
46. The method of any one of embodiments 1-5, 8-11, and 15-45, wherein the one or more indicators are measured at about 4 weeks following administration of the one or more doses of psilocybin or an active metabolite thereof.
47. The method of any one of the embodiments 1-46, wherein the subject receiving treatment shows improvement in one or more symptoms of BED or one or more symptoms of BED are ameliorated.
48. The method of any one of the embodiments 1-47, wherein the subject receiving treatment shows improvements selected from among one or more of: decreased frequency of binge eating episodes, decreased Binge Eating Scale (BES), decreased binge eating episodes per day, decreased anxiety around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased overall anxiety, decreased anxiety regarding food, improved vision of self, improved self-confidence, improvement in depression and feeling genuinely happy, lack of binge eating desires, weight loss, improved eating behavior, decreased Hospital Anxiety and Depression Scale (HADS), decreased Patient Global Impression (PGI-I), and decreased Acceptance and Action Questionnaire (AAQ-II).
49. The method of any one of the embodiments 1-48, wherein the subject receiving treatment shows improvements selected from among one or more of: decreased frequency of binge eating episodes, decreased Binge Eating Scale (BES), decreased binge eating episodes per day, decreased anxiety around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased Hospital Anxiety and Depression Scale (HADS), decreased Patient Global Impression (PGI-I), and decreased Acceptance and Action Questionnaire (AAQ-II).
50. The method of any one of the embodiments 1-48, wherein the subject receiving treatment shows improvements selected from among one or more of: decreased overall anxiety, decreased anxiety regarding food, improved vision of self, improved self-confidence, improvement in depression and feeling genuinely happy, lack of binge eating desires, weight loss, and improved eating behavior.
51. The method of any of embodiments 1-50, wherein the one or more doses of psilocybin or an active metabolite thereof comprises 1, 2, 3, 4, or 5 doses of psilocybin or an active metabolite thereof.
52. The method of any of embodiments 1-51, wherein the one or more doses of psilocybin or an active metabolite thereof comprises 1 dose of psilocybin or an active metabolite thereof.
53. The method of any of embodiments 1-51, wherein the one or more doses of psilocybin or an active metabolite thereof comprises 2 doses of psilocybin or an active metabolite thereof.
54. The method of any of embodiments 1-53, wherein the dose is between at or about 10 mg and at or about 50 mg of psilocybin or an active metabolite thereof.
55. The method of embodiment 54, wherein the dose is at or about 25 mg of psilocybin or an active metabolite thereof.
56. The method of any one of embodiments 1-55, wherein the one or more integration session comprises 1, 2, 3, 4, or 5 integration sessions.
57. The method of any of embodiments 1-56, wherein the one or more integration session comprises 1 integration session.
58. The method of any of embodiments 1-56, wherein the one or more integration session comprises 2 integration sessions.
59. The method of any of embodiments 1-58, wherein the integration session is provided at or about 1 day after administering the dose of psilocybin or an active metabolite thereof.
60. The method of any of embodiments 53-59, further comprising providing one or more further integration sessions.
61. The method of embodiment 60, wherein the one or more further integration session is provided between about 6 days and at or about 9 days after administering the dose of psilocybin or an active metabolite thereof.
62. The method of embodiment 60 or 61, wherein the one or more further integration session is provided at or about 7 days after administering the dose of psilocybin or an active metabolite thereof.

### V. EXAMPLES

The following examples are included for illustrative purposes only and are not intended to limit the scope of the invention.

### Example 1: Administration of Psilocybin and Psychotherapy in Subjects with Binge Eating Disorder (BED)

This Example describes an open-label clinical study to assess the safety and efficacy of oral psilocybin to treat human subjects with a Binge eating disorder (BED). BED is typically characterized by a severe disturbance in control of overeating behavior and high anxiety around food. Additionally, BED is associated with obesity and psychiatric comorbidities, including depression, and impulsive and compulsive disorders. The Example supports that psilocybin can be useful in the treatment of BED through a variety of mechanisms, including moderating overall anxiety, anxiety around food, perseveration, and repetitive and intrusive thoughts about food.

### A. Study Design

The subjects were administered one 25 mg dose of psilocybin orally. The safety and efficacy of psilocybin and psychotherapy were evaluated over a 12-week period following administration of a single dose of psilocybin, with the primary endpoint being at 4 weeks following administration. Subjects were screened and baseline measurements were determined prior to two preparatory sessions, which preceded the dosing session. Two integration sessions follow dosing, and therapy and safety follow-up assessments were completed for 12 weeks after the dosing session.

### B. Inclusion and Exclusion Criteria

Subjects who meet the Diagnostic and Statistical Manual of Mental Disorders, 5th Edition (DSM-5) criteria for BED were eligible for participation in the study. Prospective subjects were excluded from the study based on a variety of criteria, including but not limited to, physiological and psychological medical conditions, certain prior and/or concomitant therapies, diagnostic assessments made during subject screening or baseline evaluation, and a family history of psychosis.

Exemplary inclusion criteria included:
1. Meet Diagnostic and Statistical Manual of Mental Disorders, 5th Edition (DSM-5) criteria for BED.
2. Age ≥18 and ≤64 years.
3. Provision of signed and dated informed consent form.
4. Stated willingness to comply with all study procedures and availability for the duration of the study.
5. Medically stable in the judgment of the Principal Investigator, as determined by screening medical, physical examination, ECG, and routine laboratory tests including blood and urinalysis.
6. For females of reproductive potential: use of highly effective contraception for at least 1 month prior to screening and agreement to use such a method during study participation and for an additional 4 weeks following the dose of psilocybin. Adequate birth control methods include intrauterine device; injected, implanted, intravaginal, or transdermal hormonal method; oral hormones plus a barrier contraception; abstinence; vasectomized sole partner; or double barrier contraception.
7. For males of reproductive potential: use of condoms or other methods to ensure effective contraception with partner through 90 days post-dose.
8. Agree to consume approximately the same amount of caffeine-containing beverage (e.g., coffee, tea) that he/she consumes on a usual morning, before arriving at the research unit on the morning of the drug session day. If the participant does not routinely consume caffeinated beverages, he/she must agree not to do so on the dosing session day.
9. Agree to refrain from using any psychoactive drugs, including alcoholic beverages for a minimum of 1 week prior to drug administration.
10. Agree that for 1 week before the drug session, including the morning of the session, he/she will refrain from taking any nonprescription medication, nutritional supplement, herbal supplement, or as needed (PRN) prescription medication except when approved by the study investigators. Exceptions will be evaluated by the study investigators and will include acetaminophen, non-steroidal anti-inflammatory drugs, and common doses of vitamins and minerals and contraceptives.

Exemplary exclusion criteria included:
1. Significant suicide risk as defined by either suicidal ideation as endorsed on items 4 or 5 on the C-SSRS within the past year, at screening, or at baseline; or suicidal behaviors within the past year; clinical assessment of significant suicidal risk during subject interview. The C-SSRS is a questionnaire that was designed to distinguish the domains of suicidal ideation and suicidal behavior through four constructs, severity of ideation, intensity of ideation, behavior subscale, and the lethality subscale (Posner et al., American Journal of Psychiatry, 2011; 168(12):1266-1277).
2. Participation in another concurrent clinical study or within the preceding month.
3. Women who are pregnant or who intend to become pregnant during the study or who are currently nursing.
4. Vital signs, averaged over 3 readings within 15 minutes, of systolic blood pressure (BP)>139 mm Hg, diastolic BP>89 mm Hg, or heart rate >90 bpm.
5. Have any of the following cardiovascular conditions: uncontrolled hypertension, coronary artery disease, congenital long QT syndrome, cardiac hypertrophy, cardiac ischemia, congestive heart failure, prior myocardial infarction, tachycardia, artificial heart valve, QTc>450 msec at screening, any other clinically significant screening ECG abnormality, or any other significant cardiovascular condition.
6. Presence of a gastrointestinal disease that could interfere with absorption of orally-administered psilocybin.
7. Have epilepsy.
8. Meet DSM-5 criteria for schizophrenia spectrum or other psychotic disorders, including major depressive disorder with psychotic features, or Bipolar I or Bipolar II Disorder.
9. Family history of psychosis.
10. Meet DSM-5 criteria for a moderate or severe alcohol or drug use disorder.
11. Positive urine drug screen or alcohol breath test at screening. A repeat test can be conducted at screening or Day −1 at the discretion of the Principal Investigator or delegate.
12. Prior adverse effects from psilocybin.
13. Currently taking or expected to need prior to the dosing session, UGT1A9 or 1A10 inhibitors (e.g., regorafenib, rifampicin, phenytoin, eltrombopag, mefenamic acid, diflunisal, niflumic acid, sorafenib, isavuconazole, deferasirox, Ginseng) and aldehyde or alcohol dehydrogenase inhibitor (e.g., disulfiram).
14. Currently taking or testing positive on urine drug screen, drugs of abuse such as amphetamines, buprenorphine, benzodiazepines, cocaine, methamphetamines, Ecstasy (MDMA), morphine, methadone, oxycodone, marijuana, ethyl glucuronide, fentanyl, tramadol, and synthetic cannabinoids (K2).
15. Currently taking on a regular (e.g., daily) basis any medications having a primary centrally-acting serotonergic effect, including SSRIs, MAOIs, or serotonin-acting dietary supplements (such as 5 hydroxytryptophan or St. John's wort). For individuals who have intermittent or PRN use of such medications, the dosing session will not be conducted until at least 5 half-lives of the agent have elapsed after the last dose.
16. fMRI subjects: Contraindications to fMRI procedures, per institutional policy.

### C. Screening and Baseline Measurements

Subjects attended at least one visit for screening. The screening period (Weeks −5 and −4) included hematology, blood chemistry, urine drug screen, physical examination, and other safety assessments to evaluate medical inclusion criteria. Responses to questionnaires, including the C-SSRS, Binge Eating Scale (BES), Eating Questionnaire (Fairbum and Beglin, Int J Eat Disord, 1994; 16(4):363-370), Clinical Global Impressions Scale (CGI-I), Patient Global Impression of Improvement (PGI-I), Hospital Anxiety and Depression Scale (HADS), and Acceptance and Action Questionnaire (AAQ-II), were collected during this period. Assessments conducted during the screening period were considered baseline values for the purpose of determining change from baseline (CFB).

Daily binge eating episodes (e.g., number of binge eating episodes per day) were assessed daily over a two-week period (Weeks −3 and -2) using a two-item Eating Questionnaire. Daily binge eating episodes (e.g., number of binge eating episodes per day) were assessed prior to initiating the psychotherapy and prior to administration of the psilocybin. As an example, a subject was asked to record the number of binge eating episodes once a day using the following prompts: 1. “Over the past 24 hours, how many times have you eaten what other people would regard as an unusually large amount of food (given the circumstances)?,” and 2. “On how many of these times did you have a sense of having lost control over your eating (at the time that you were eating)?” These evaluations were considered baseline values for the purpose of determining CFB in binge eating frequency.

Baseline electroencephalogram (EEG) and fMRI measurements were conducted during the week prior to preparatory sessions for the purpose of determining CFB (Week −1). Ten minutes each of eyes open and eyes closed resting EEG was acquired in counterbalanced order. For fMRI imaging, subjects were positioned head-first in the supine position in an MRI scanner with a 64-channel head coil. Subjects rated their current level of hunger using a 100-point visual analog scale (VAS) anchored from “Not at all hungry” to “Hungriest imaginable.” A blood oxygen level-dependent (BOLD) resting state functional scan, approximately 9 minutes, was acquired first. Subjects were instructed to stay as still as possible, let their thoughts wander, keep their eyes fixated on a central cross displayed on a display, and do their best to stay awake. Next, subjects completed a food cue reactivity task during BOLD functional imaging acquisition. During this task, subjects viewed images of highly-processed foods, for example, pizza, images of minimally-processed foods, for example, an apple, and neutrally valent pictures of household objects, for example, a light bulb. Subjects were instructed to think about how much they desire each item. After task completion, the subject was withdrawn from the MRI bore and provided with a small meal. Once complete, the subject was repositioned in the MRI, and the hunger VAS, resting-state scan and cue reactivity task was repeated. The order of the resting state scans and cue reactivity task were counterbalanced. A high-resolution T1-weighted structural brain image (˜4 minutes) was then collected.

### D. Preparatory Sessions

Two preparatory sessions were held with therapists during the week leading up to the dosing session. Preparation sessions were designed to build rapport with the therapists who will be present during the psilocybin session and to identify personal themes and struggles that were likely to impact the session experience. During the first preparation session, which occurred about one week prior to the dosing session, the therapist aimed to establish a therapeutic alliance by building rapport and listening to the subject's narrative of binge eating, anxiety, guilt, and treatment history to understand the most prominent patterns of psychological inflexibility. The therapist provided psychoeducation regarding the psilocybin experience and Acceptance and Commitment Therapy (ACT), a therapeutic approach that aims to promote psychological flexibility by practicing acceptance and mindfulness (Zhang et al., Front Psychol. 2018 Jan. 11; 8:2350).

The second preparatory session occurred one to three days in advance of the dosing session. During this session, the therapists teach grounding techniques, such as breathing techniques. Therapeutic boundaries, for example, touch and safety measures were discussed. The subject was assisted in setting a motivation for the psilocybin session. Both preparation sessions were either held in-person or via video.

### E. Psilocybin Administration

Subjects were instructed to consume a low-fat breakfast at least one hour before reporting to the dosing session, where a single 25 mg oral dose of psilocybin was to be administered (Usona Institute). On dosing day, vital signs, for example, heart rate and BP, were assessed prior to administration of psilocybin and at 30, 60, 90, 120, 180, 240, 300, and 360 minutes post-administration. 12-lead ECGs were performed in triplicate pre-dosing and 60, 120, and 360 minutes after capsule administration. C-SSRS responses were reported prior to and following dosing. Throughout the session, in accordance with a supportive stance, the therapist did not provide significant ACT interventions or feedback. ACT-based clinical formulation continues as the therapist listens to emergent narratives and notes instances of psychological flexibility and inflexibility, especially present moment awareness, avoidance, and values.

Session monitors were trained to provide support to subjects if they experience acute anxiety, agitation, paranoia, or panic. However, in the event of an AE, a physician was consulted. AEs include psychiatric emergency, hypertension, acute chest pain, and headache. An oral benzodiazepine, for example, lorazepam, was available for treatment of panic or anxiety. An oral antipsychotic, for example, risperidone, was available for treatment of psychosis or severe agitation. If a subject experienced hypertension, a BP of >200 mm Hg systolic or >110 mm Hg diastolic for more than 15 minutes from four consecutive readings, the subject was transferred to the emergency room whether or not they were symptomatic. Labetalol or similar drug was available for treatment of hypertension. If a subject developed new-onset chest pain, the subject was evaluated in-person by the study physician to determine whether the pain was cardiovascular or musculoskeletal, or reflects acute panic/anxiety. Vital signs and ECG were monitored and compared to baseline measurements for evidence of acute ischemia. Ibuprofen or a similar medication was available for treatment of a headache.

### F. Integration Sessions

After the dosing session, subjects were engaged in two integration sessions. The first session was held in-person one day after dosing, and the second integration session was held by video or in-person approximately 7 days after dosing. During the integration sessions, focused ACT-based clinical activity, related to contacting the present moment, defusion, or acceptance based on clinical formulation, was practiced. Additionally, the subject's psilocybin experience and any emotional, mental, or lifestyle changes that followed the dosing session were reviewed and reflected upon.

### G. Follow-Up Sessions

An initial therapy follow-up session was held approximately two weeks after the psilocybin dosing session (Week 4). During the follow-up session, the therapist continued to explore and reinforce insights gained from the psilocybin experience, while assessing for changes in psychological flexibility. The subject and therapist reflected upon how the dosing and therapy sessions brought each ACT process to light, and whether any successful behavioral changes and committed actions were taken. Mindfulness practices and other concrete ways the study experience can be translated into lasting changes were discussed, and the therapist led a discussion on plans for follow-up care for the subject.

Safety follow-ups were held at Weeks 4, 5, 8, and 12 (2, 3, 6, and 10 weeks after the dosing session). Safety follow-up sessions include assessments of binge eating behavior, such as with use of the C-SSRS and the Eating Questionnaire. In-person follow-up visits were scheduled to be held at Weeks 6, 10, and 14 (4, 8, and 12 weeks after dosing). During in-person follow-ups several evaluations are made, including physical examination, such as height, weight, BMI, and waist circumference measurements, and assessment of vital signs and metabolic indicators. Additionally, fMRI and EEG are performed. Responses to the C-SSRS, BES, Eating Questionnaire (daily and over the course of 4 weeks), CGI-I, PGI-I, HADS, and AAQ-II are also assessed.

### H. Study Endpoints

Regarding analysis and presentation of collected data, continuous data were summarized by the number of subjects with no-missing data (n), mean, median, SD, minimum, and maximum values. For categorical variables, the number and percent of each category was displayed. Indicators or biomarkers were set up to be analyzed using the appropriate statistical methodology for the given endpoint type and objective.

Endpoints included determining the effects of psilocybin on indicators of clinical activity in BED population throughout the study and assessing the relationship between clinical activity and the intensity of the psychedelic experience. Indicators were determined using a variety of assessments, including evaluations of binge eating behavior, physical examination, subject-reported outcomes, neurophysiologic indicators, and metabolic indicators. Exploratory analyses include CFB at all tested time points for such parameters.

For example, binge eating behavior, such as binge eating episodes, was assessed using questionnaires, such as the Eating Questionnaire. The observer-reported CGI-I, a 7-point scale that requires a clinician to assess how much the subject's illness has improved or worsened relative to the baseline state, was also administered to determine, among other factors, symptom severity and treatment response. Several other indicators were assessed using subject-reported questionnaires, including, for example, the BES, PGI-I, EBI, HADS, and AAQ-II, such as at 4, 8, and 12 weeks following dosing.

Safety was planned for evaluation for one dose of psilocybin in subjects with BED during the dosing session and through 12 weeks following dosing (Week 14). Several assessments were made to determine safety, including evaluating the nature and severity of AEs, physically examining subjects, monitoring electrocardiogram (ECG) and changes in vital signs (e.g., blood pressure and heart rate), measuring clinical laboratory parameters (e.g., hematology and blood chemistry), and evaluating responses to the C-SSRS through 12 weeks following dosing.

Vital signs, such as BP and heart rate, were recorded at the dosing session, the day following dosing, and at Weeks 6, 10, and 14 (4, 8, and 12 weeks after the dosing session). Physical examination was reported at screening, the day following dosing, and at Weeks 6, 10, and 14. ECG, blood chemistry, and hematology were tabulated at screening, at dosing, at the first integration session, and at Weeks 6, 10, and 14. Blood chemistry panels include measurement of Na⁺, K⁺, Cl⁻, HCO₃⁻, Ca²⁺, Mg²⁺, P, blood urea nitrogen (BUN), creatinine, glucose, total bilirubin, albumin, ALT, AST, GGT, CK, LDH, and alkaline phosphatase. Hematology evaluations include CBC with white cell differential and platelet count. Urinalysis includes assessment of pH, specific gravity, protein, occult blood, glucose, and ketones plus microscopic examination of sediment for RBC, WBC, epithelial cells, casts, crystals, and bacteria.

Adverse events (AEs) were monitored throughout the study. An AE can be any unfavorable and unintended, symptom, or disease temporarily associated with the use of a drug. No judgement on causality is implied with use of the term. AEs are graded in severity from mild to severe. Mild AEs require minimal or no treatment and do not interfere with the subject's daily activities. Moderate AEs result in a low level of inconvenience or concern with the therapeutic measures. However, moderate events may cause some interference with functioning. Severe AEs interrupt a subject's usual daily activity and may require systemic drug therapy or other treatment. Severe events are usually potentially life-threatening or incapacitating.

Other endpoints included assessing one or more indicators related to the psychedelic state, such as evaluating the feasibility of inducing the psychedelic state with psilocybin in BED population and determining the preliminary clinical activity and the effects of psilocybin in conjunction with psychotherapy on the frequency of binge-eating episodes and other weight-related indicators in BED population through four weeks following dosing (i.e., Week 6). The Mystical Experience Questionnaire-30 item (MEQ30) and Monitor Rating Scale (MRS) were used to assess the magnitude and duration of psilocybin-induced dissociative effects in subjects with BED. Baseline levels of frequency of binge eating episodes, CGI-I, waist circumference, and body mass index (BMI) were compared to measurements at four weeks post-dosing to determine CFB. The relationship between clinical activity, such as a clinical response to psilocybin, and the intensity of the psychedelic experience was assessed. The intensity of the psychedelic experience, such as the magnitude and duration of dissociative effects, was evaluated using a variety of questionnaires including the MEQ30, the Challenging Experiences Questionnaire (CEQ), Emotional Breakthrough Inventory (EBI), and the MRS.

Physical examination included, for example, measuring waist circumference and BMI, such as at 8 and 12 weeks following dosing. Determination of metabolic indicators include, for example, assessments of ghrelin, leptin, adiponectin, insulin, glucose, and HOMA-IR (Homeostatic Model Assessment of Insulin Resistance), such as at 4, 8, and 12 weeks following dosing. Evaluations of neurophysiologic indicators included various fMRI evaluations, including, resting state functional connectivity (fed and fasted), task-related functional activation and connectivity during a food cue reactivity task (fed and fasted), voxel-based morphometry (grey matter volume/structure), and EEG, for example, explosive synchronization of network activity at rest and power spectrum analysis (frequency bands), such as at 4 weeks post-dosing.

### I. Results

In one subject, immediate changes were observed after administration of the psilocybin. The subject reduced interest in eating and was resistant to the trigger foods, and facial expressions indicative of anxiety (e.g., constant furrow in the brows) disappeared. Improvements, including decreased overall anxiety, decreased anxiety regarding food, improved vision of self, improved self-confidence, improvement in depression and feeling genuinely happy, lack of binge eating desires, weight loss, and improved eating behavior, relating to symptoms or behaviors associated with BED were observed.

Table 4 describes the changes in BMI (kg/m²) and waist circumference (cm) in a subject treated as described. As shown, the BMI and waist circumference decreased in this subject, at 27 days after administration of psilocybin.

TABLE 4
Changes in BMI and Waist Circumference

| | | Waist |
|---|---|---|
| Time relative to Dosing Day (days) | BMI (kg/m²) | circumference (cm) |
| −42 | 42.9 | 106.6 |
| 0 (day of administration | 43.5 | 105.9 |
| of psilocybin) | | |
| 27 | 42.5 | 104.4 |

The Binge Eating Scale (BES) score from the subject was 13 at day −42, and 5 at day 27. BES ranges from 0 to 46, with a higher score associated with BED symptoms.

Table 5 describes the changes in Hospital Anxiety and Depression Scale (HADS) score of the subject. As shown, the depression and anxiety scores both were reduced at 13 or 27 days after administration of psilocybin, consistent with a reduction in overall depression and anxiety.

TABLE 5
Changes in HADS

| | Depression | |
|---|---|---|
| Time relative to Dosing Day (days) | Score | Anxiety Score |
| −42 | 5 | 5 |
| 0 (day of administration of psilocybin, | 5 | 5 |
| before dosing) | | |
| 0 (day of administration of psilocybin, | 6 | 6 |
| after dosing) | | |
| 13 | 0 | 4 |
| 27 | 1 | 2 |

The Patient Global Impression (PGI-I) was measured relative to the seven prior days (including the day of the visit up to and through the visit) compared to the patient's perceived condition at baseline visit. The patient rated the condition based on the following: 1. Very much improved since baseline (initiation of treatment), 2. Much improved, 3. Minimally improved, 4. No change from baseline, 5. Minimally worse, 6. Much worse, 7. Very much worse from baseline. For this subject, the PGI-I at day −42 (42 days before administration of psilocybin) was set at 4, as the baseline. The subject reported a score of 1 on day 13, and 1 on day 27 after administration of psilocybin, indicating substantial patient perceived improvement of the condition.

The Acceptance and Action Questionnaire (AAQ-II) score was 13 at day −42, 10 at day 13, and 10 at day 27, after administration of psilocybin. Higher AAQ-II score correspond to lower psychological well-being. The results were consistent with improved overall psychological well-being.

The daily eating questionnaire included question 1 (Q1) “Over the past 24 hours, how many times have you eaten what other people would regard as an unusually large amount of food (given the circumstances)?”, question 2 (Q2) “On how many of these times did you have a sense of having lost control over your eating (at the time that you were eating)?” and question 3 (Q3): “Over the past 28 days, on how many DAYS have such episodes of overeating occurred (i.e., you have eaten an unusually large amount of food and have had a sense of loss of control at the time)?”

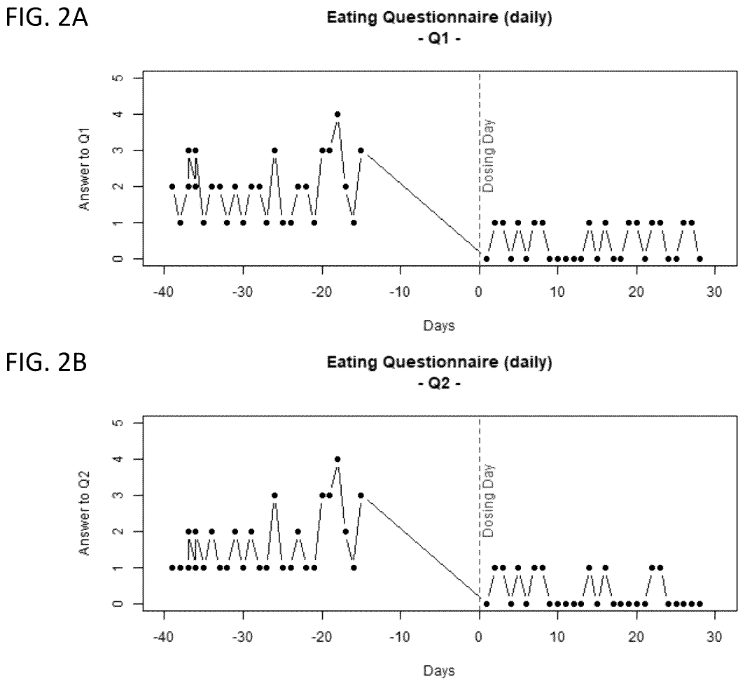

As depicted in FIG. 2A (Q1) and FIG. 2B (Q2), the answers for Q1 and Q2 decreased overall in the days after the administration of psilocybin.

The Challenging Experience Questionnaire scores (reported one day after dosing) were as follows:
Fear: 0.16
Grief: 0
Physical Distress: 0.24
Insanity: 0
Isolation: 0
Death: 0
Paranoia: 0
Total Score: 0.06

CEQ ranges from 0.000 to 1.000 and a high score corresponds to important acute adverse psychological reactions to drug (“bad trips” or “challenging experiences”).

The Mystical Experience Questionnaire (MEQ30) scores reported one day after dosing were as follows:
Transcendence of time and space: 46.67%
Positive mood: 93.33%
Ineffability: 80%
Mystical: 45.33%
Total Score: 58.67%

MEQ30 ranges from 0% to 100%, and a high score corresponds to an intense mystical-type experience.

No adverse events were observed during the administration or the integration sessions. No substantial changes were observed in heart rate or blood pressure.

The results from the one subject immediately following the post-dosing integration session with psychotherapists and throughout the four-week period post-dosing showed that the subject exhibited reduced overall anxiety, reduced anxiety around food, reduced compulsion to overeat and improved self-image and confidence.

At a further assessment point of the clinical study, five (5) total subjects were assessed after administration of psilocybin. Analysis of the additional four subjects were consistent with the clinical observations seen for the first subject as described above.

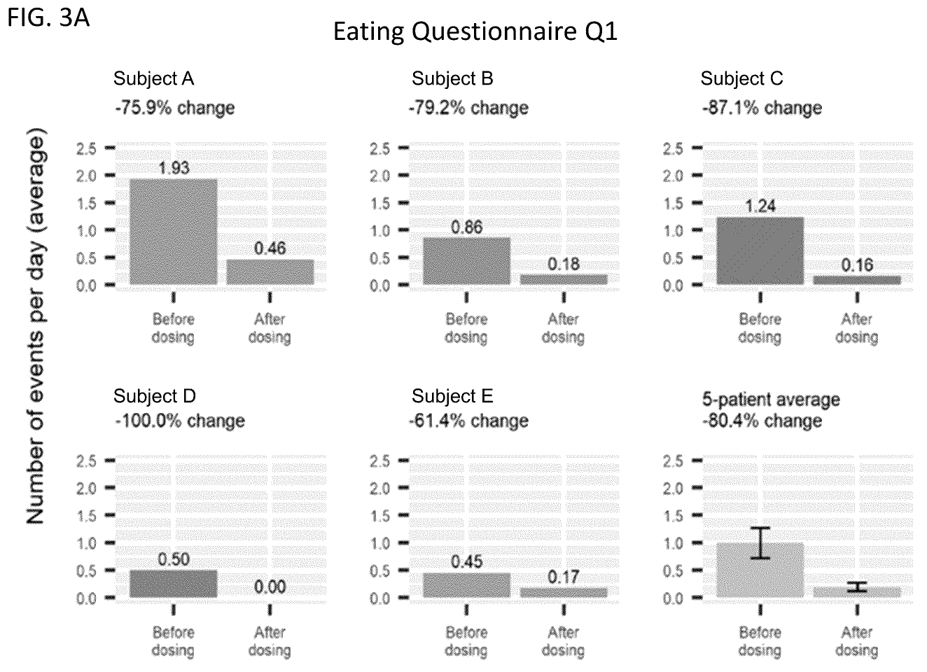

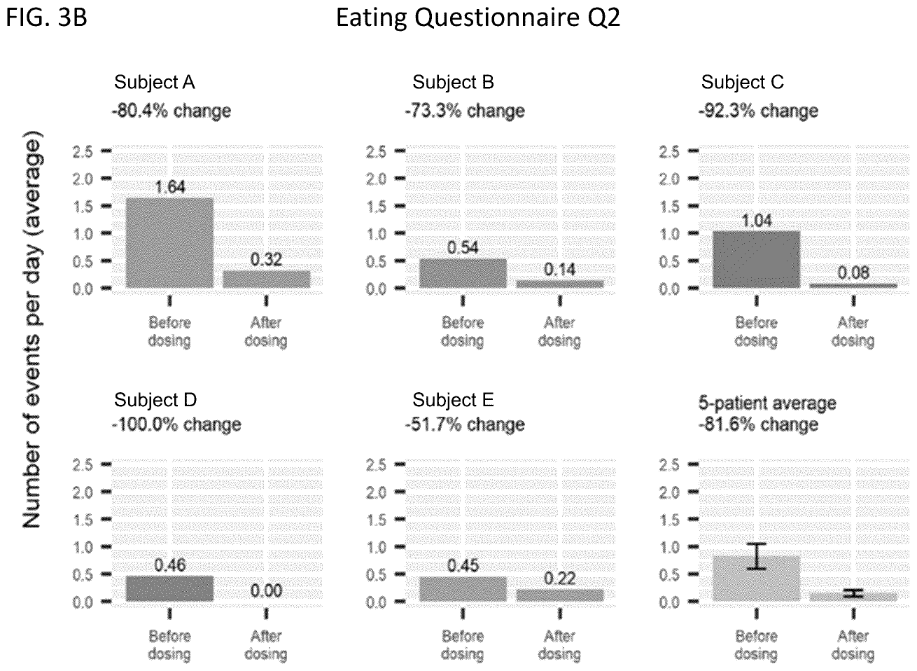

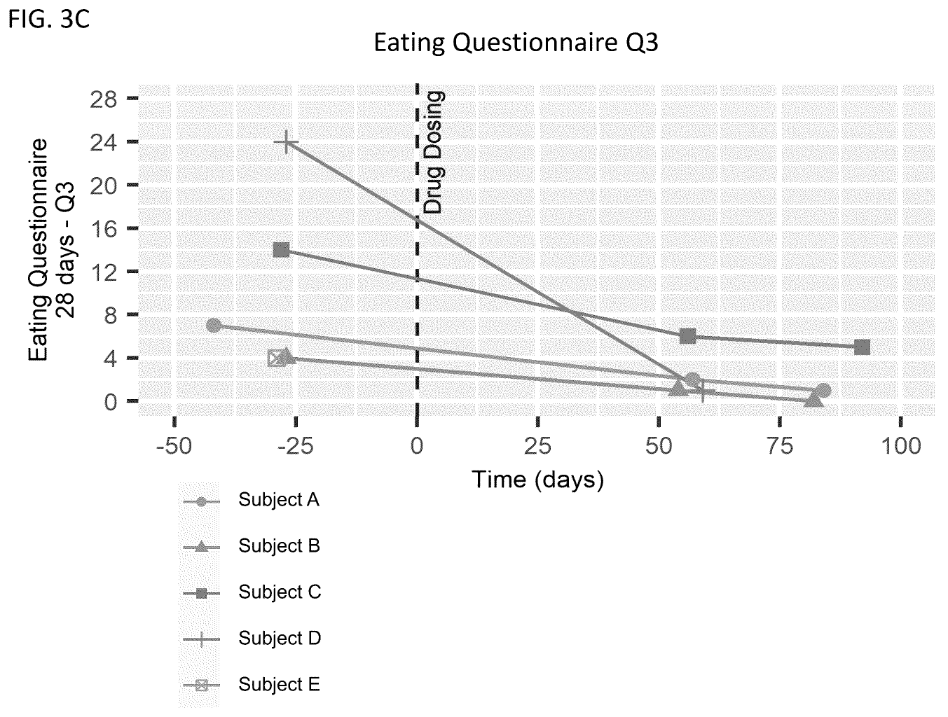

As depicted in FIG. 3A (Eating Questionnaire Q1) and FIG. 3B (Eating Questionnaire Q2), the number of events per day (average) for Q1 and Q2 decreased after the administration of psilocybin in all subjects. Across the 5 subjects, daily binge eating episodes were reduced by an average of 80.4% from baseline during the four-week post-dosing measurement period, with all subjects reporting a daily reduction in binge eating episodes of at least 60% from baseline. As shown in FIG. 3A, 4 of 5 subjects reported at least a 75% reduction in daily binge eating episodes from baseline during the four-week post-dosing measurement period. As shown in in FIG. 3B, the number of daily instances of subjects feeling that they had lost control over their eating were reduced by an average of 81.6% during the four-week post-dosing measurement period, with 4 of 5 subjects reporting a reduction of greater than 70%. As shown in FIG. 3C (Eating Questionnaire Q3), the frequency of binge eating (FBE) evaluation (i.e., the number of days among the past 28 days that a binge eating episodes occurred), also decreased substantially in all of the tested subjects, and the decrease persisted until 50 or more days after administration in all 4 subjects for whom data was available. For 3 subjects for whom data was available, the decrease persisted for over 80 days.

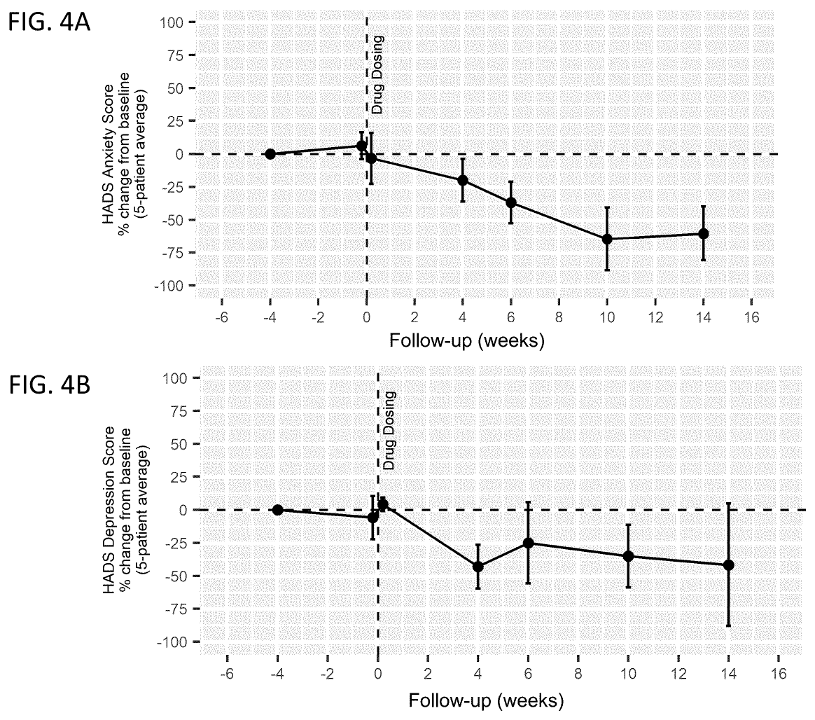

Analysis of the Hospital Anxiety and Depression Scale (HADS) anxiety (FIG. 4A) and depression (FIG. 4B) scores demonstrated improving trends related to the subjects' levels of anxiety and depression. No drug-related adverse events were reported by the subjects during the four-week period following administration.

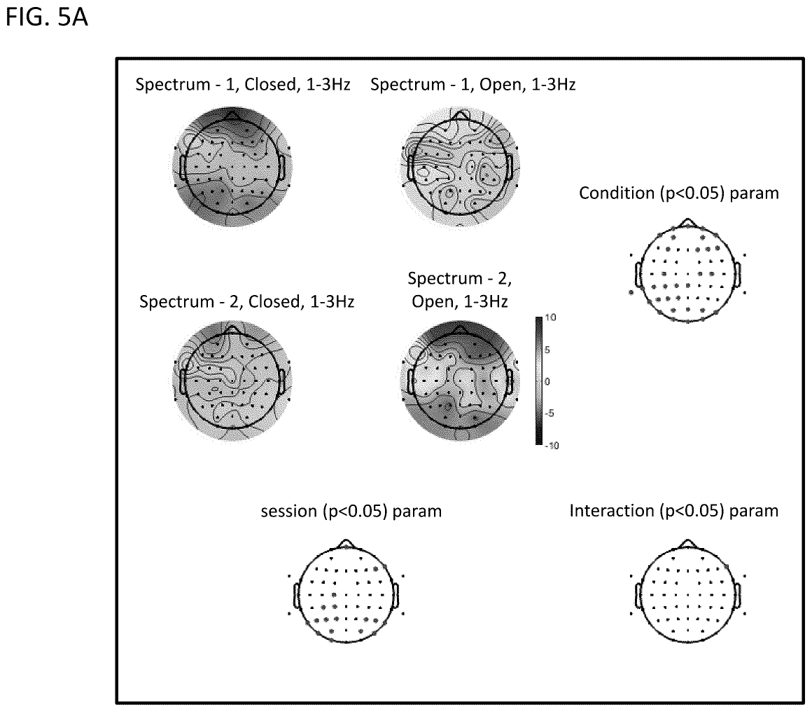

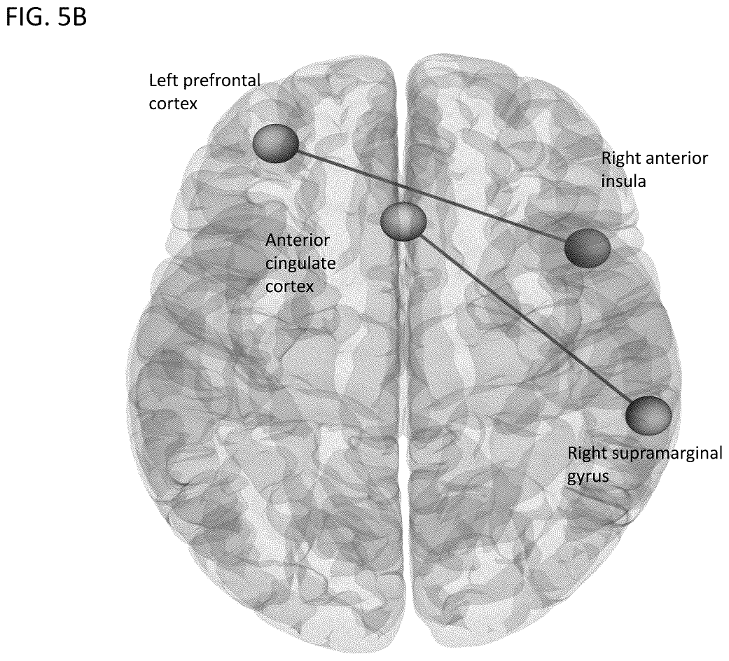

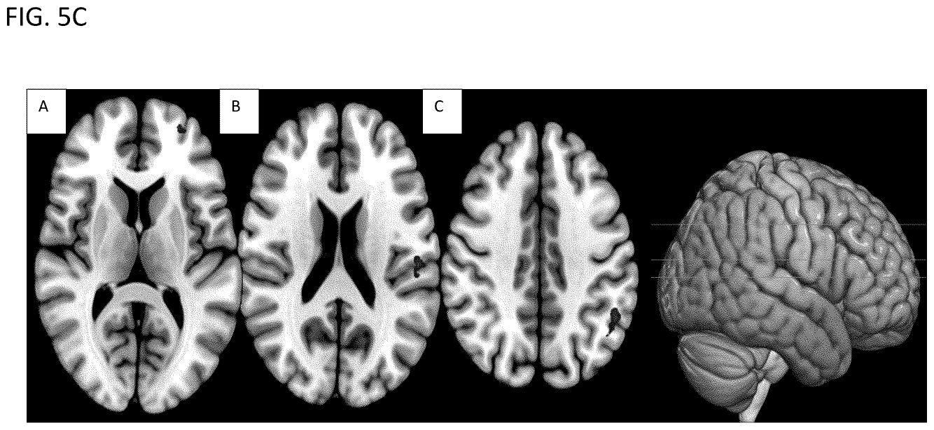

EEG and fMRI-based neuroimaging analyses focused primarily on the changes in brain function and connectivity at rest from pre- to post-treatment with psilocybin-assisted psychotherapy as described above. For EEG, medium-to-large alterations were observed in resting connectivity across the whole brain over the delta, theta, alpha, and beta frequency bands. Specifically, decreases in resting connectivity were predominately observed. In addition, power in posterior electrodes was observed to increase from pre- to post-treatment in the delta, theta, and beta frequency bands. Specifically, in both the eyes open and eyes closed condition, spectral power in the delta band increased from pre- to -post-treatment. (FIG. 5A). Without wishing to be bound by theory, it is considered that greater power within slower spectral bands (delta and theta) is related to more effective emotion regulation. Therefore, in some aspects, the observed greater power within slower spectral bands is associated with the beneficial effects of psilocybin-assisted psychotherapy for affective symptoms associated with BED described herein.

For fMRI, changes from pre- to post-treatment on connectivity within critical resting state brain networks, including default mode network (DMN), salience network (SN), and executive control network (ECN) were assessed. Additionally, connectivity of these networks with the rest of the brain both at rest and after presentation of processed vs. unprocessed food and non-food cues was assessed. Connectivity between brain areas comprising the SN following presentation of processed foods vs. unprocessed foods was observed to increase from pre- to post-treatment, as well as between ECN and SN with structures involving vision processing and memory function (FIG. 5B). Prior to treatment, processed food cue presentation evoked lower connectivity between nodes than unprocessed cues. After treatment, the opposite pattern was observed.

In addition, connectivity of these networks with other areas of the brain associated with processing bodily sensations and vision at rest was altered following treatment. Finally, patterns of brain activation following cue presentation was also assayed. Here, it was observed that activity in areas associated with cognitive control was greater following presentation of processed food cues after treatment vs. before treatment (FIG. 5C). Several clusters were identified where presentation of processed food cues was associated with lower activation than unprocessed food cues prior to treatment, but greater activation at post-treatment follow-up. These included (a) left middle frontal gyrus, (b) left supramarginal gyrus, and (c) left angular gyrus. Overall, the fMRI results described above suggest that psilocybin-assisted psychotherapy may alter functional activation and network connectivity associated with processing of processed and unprocessed food cues.

The results showed that administration of psilocybin together with psychotherapy resulted in significant improvement of several different symptoms and indicators associated with BED, such as the frequency of binge eating behavior, without adverse events. The results showed that a single dose of psilocybin combined with psychotherapy led to substantial improvements in BED symptoms and quality of life, and the magnitude of changes for most subjects in binge eating, anxiety, and depression were dramatic. Such magnitudes are typically only observable after much longer periods of evidence-based therapy. The results also showed that the effects on BED symptom improvement can last for 80 days or more after administration of psilocybin, demonstrating a durable and lasting therapeutic effect even after administration of a single dose of the psychedelic. The results support the utility of the methods and uses involving the administration of psychedelics such as psilocybin or psilocin for treating BED.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating binge eating disorder (BED), the method comprising:
orally administering to a subject suffering from one or more symptoms of BED, one or more doses of psilocybin or an active metabolite thereof effective to induce a dissociative state;
providing one or more integration sessions to the subject after emergence from the dissociative state; and
assessing in the subject one or more indicators prior to and/or after administration of the one or more doses of psilocybin or an active metabolite thereof; wherein the one or more indicators are associated with the one or more symptoms of BED, dissociative state, treatment outcome, safety and/or is related to the subject.

2. A method of treating binge eating disorder (BED), the method comprising assessing in the subject one or more indicators prior to and/or after administration of the one or more doses of psilocybin or an active metabolite thereof in a subject suffering from one or more symptoms of BED that has been provided one or more integration sessions after emergence from a dissociative state induced by orally administered one or more doses of psilocybin or an active metabolite thereof effective to induce a dissociative state; wherein one or more indicators associated with the one or more symptoms of BED, dissociative state, treatment outcome, safety and/or is related to the subject are assessed in the subject prior to and/or after administration of the one or more doses of psilocybin or an active metabolite thereof.

3. The method of claim 1 or 2, wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).

4. A method of treating binge eating disorder (BED), the method comprising:
orally administering to a subject suffering from one or more symptoms of BED, one or more doses of psilocybin or an active metabolite thereof effective to induce a dissociative state;
providing one or more integration sessions to the subject after emergence from the dissociative state; and
assessing in the subject one or more indicators prior to and/or after administration of the one or more doses of psilocybin or an active metabolite thereof; wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).

5. A method of treating binge eating disorder (BED), the method comprising assessing in the subject one or more indicators prior to and/or after administration of the one or more doses of psilocybin or an active metabolite thereof in a subject suffering from one or more symptoms of BED that has been provided one or more integration sessions after emergence from a dissociative state induced by orally administered one or more doses of psilocybin or an active metabolite thereof effective to induce a dissociative state; wherein one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II) are assessed in the subject prior to and/or after administration of the one or more doses of psilocybin or an active metabolite thereof.

6. A method of treating binge eating disorder (BED), the method comprising:
orally administering to a subject suffering from one or more symptoms of BED, one or more doses of psilocybin or an active metabolite thereof effective to induce a dissociative state; and
providing one or more integration sessions to the subject after emergence from the dissociative state.

7. A method of treating binge eating disorder (BED), the method comprising providing one or more integration sessions a subject suffering from one or more symptoms of BED after emergence from a dissociative state induced by orally administered one or more doses of psilocybin or an active metabolite thereof effective to induce a dissociative state.

8. The method of claim 6 or 7, further comprising assessing in the subject one or more indicators prior to and/or after administration of the one or more doses of psilocybin or an active metabolite thereof.

9. The method of claim 8, wherein the one or more indicators are associated with the one or more symptoms of BED, dissociative state, treatment outcome, safety and/or is related to the subject.

10. The method of any of claims 1-9, further comprising, prior to administering the one or more doses of psilocybin or an active metabolite thereof, assessing one or more of the following parameters in the subject:
(1) risk of suicide;
(2) vital signs;
(3) incidence of cardiovascular conditions;
(4) incidence of gastrointestinal disease;
(5) incidence of epilepsy;
(6) incidence of schizophrenia spectrum or other psychotic disorders;
(7) family history of psychosis;
(8) a moderate or severe alcohol or drug use disorder;
(9) prior adverse effects from psilocybin or an active metabolite thereof; and
(10) use of prior or concomitant medications.

11. The method of claim 10, further comprising identifying the subject for treatment with psilocybin or an active metabolite thereof if the subject meets the criteria for the one or more parameters.

12. A method of identifying a subject for treating binge eating disorder (BED) with psilocybin or an active metabolite thereof, the method comprising:
(a) assessing in a subject suffering from one or more symptoms of BED, one or more of the following parameters:
(1) risk of suicide;
(2) vital signs;
(3) cardiovascular conditions;
(4) presence of gastrointestinal disease;
(5) incidence of epilepsy;
(6) incidence of schizophrenia spectrum or other psychotic disorders;
(7) family history of psychosis;
(8) a moderate or severe alcohol or drug use disorder;
(9) prior adverse effects from psilocybin or an active metabolite thereof; and
(10) use of prior or concomitant medications;
(b) identifying the subject for treatment with one or more doses of psilocybin or an active metabolite thereof if the subject meets a criteria for the one or more parameters.

13. A method of identifying a subject for treating binge eating disorder (BED) with one or more doses of psilocybin or an active metabolite thereof, the method comprising identifying a subject suffering from one or more symptoms of BED for treatment with psilocybin or an active metabolite thereof if the subject meets a criteria for one or more of the following parameters that are assessed in the subject:
(1) risk of suicide;
(2) vital signs;
(3) cardiovascular conditions;
(4) presence of gastrointestinal disease;
(5) incidence of epilepsy;
(6) incidence of schizophrenia spectrum or other psychotic disorders;
(7) family history of psychosis;
(8) a moderate or severe alcohol or drug use disorder;
(9) prior adverse effects from psilocybin or an active metabolite thereof; and
(10) use of prior or concomitant medications.

14. The method of claim 12 or 13, further comprising:
orally administering to the identified subject, one or more doses of psilocybin or an active metabolite thereof effective to induce a dissociative state; and
providing one or more integration sessions to the subject after emergence from the dissociative state.

15. The method of any of claims 6-14, further comprising assessing in the subject one or more indicators prior to and/or after administration of the one or more doses of psilocybin or an active metabolite thereof.

16. The method of claim 15, wherein the one or more indicators are associated with the one or more symptoms of BED, dissociative state, treatment outcome, safety and/or is related to the subject.

17. The method of any of claims 1, 2, 8-11, 15, and 16, wherein the one or more indicators comprise observer-rated parameters, subject-reported parameters and/or clinical indicators.

18. The method of claim 1-17, wherein the one or more indicators are assessed prior to the administration as a baseline measurement, and after the administration as an outcome measurement.

19. The method of claim 18, wherein the changes between the baseline measurement and the outcome measurement are determined.

20. The method of claim 19, wherein the subject exhibits amelioration of the one or more indicators between the baseline measurement and the outcome measurement.

21. The method of any of claims 1, 2, 8-11, and 15-20, wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, body mass index (BMI), overall anxiety, anxiety regarding food, vision of self, self-confidence, feeling genuinely happy, binge eating desires, weight, eating behavior, Hospital Anxiety and Depression Scale (HADS), Patient Global Impression (PGI-I), and Acceptance and Action Questionnaire (AAQ-II).

22. The method of any of claims 1-5, 8-11, and 15-21, wherein the one or more indicators are selected from among frequency of binge eating episodes, Binge Eating Scale (BES), binge eating episodes per day, anxiety around food, depression about eating behavior, depression about control of weight, Clinician Global Impression—Improvement (CGI-I) scale, interest in eating trigger foods, waist circumference, and body mass index (BMI).

23. The method of any of claims 1-5, 8-11, and 15-22, wherein the one or more indicators comprises frequency of binge eating episodes.

24. The method of any of claims 1-5, 8-11, and 15-23, wherein the frequency of binge eating episodes is measured daily.

25. The method of any of claims 1-5, 8-11, and 15-24, wherein the one or more indicators comprises the number of binge eating episodes per day.

26. The method of any of claims 1-5, 8-11, and 15-25, wherein the frequency of binge eating episodes is determined over the 28 days prior to the date of measurement.

27. The method of any of claims 1-5, 8-11, and 15-26, wherein the one or more indicators comprises the frequency of binge eating episodes in the 28 days prior to the date of measurement.

28. The method of any of claims 1-5, 8-11, and 15-27, wherein the one or more indicators comprises frequency of binge eating desires as indicated by the sense of loss of control over eating.

29. The method of any of claims 1-5, 8-11, and 15-28, wherein the one or more indicators comprises the frequency of sense of loss of control over eating per day.

30. The method of any of claims 1-5, 8-11, and 15-29, wherein the one or more indicators comprises interest in eating trigger foods.

31. The method of any of claims 1-5, 8-11, and 15-30, wherein the one or more indicators is measured using the Eating Questionnaire.

32. The method of any of claims 1-5, 8-11, and 15-31, wherein the one or more indicators are selected from among Emotional Breakthrough Inventory (EBI), Acceptance and Action Questionnaire-II (AAQ-II), Patient Global Impression-Improvement (PGI-I), and Hospital Anxiety and Depression Scale (HADS).

33. The method of any of claims 1-5, 8-11, and 15-32, wherein the one or more indicators is HADS—anxiety.

34. The method of any of claims 1-5, 8-11, and 15-33, wherein the one or more indicators is HADS—depression.

35. The method of any of claims 1-5, 8-11, and 15-34, wherein the one or more indicators are one or more neurophysiologic biomarkers selected from among Resting state functional connectivity (fed and fasted) by functional magnetic resonance imaging (fMRI), task-related functional activation and connectivity during a food cue reactivity task (fed and fasted) by fMRI, voxel-based morphometry (grey matter volume/structure) by fMRI, and resting electroencephalogram (EEG).

36. The method of any of claims 1-5, 8-11, and 15-35, wherein the one or more indicators are one or more metabolic biomarkers selected from among ghrelin, leptin, adiponectin, insulin, glucose, and HOMA-IR (Homeostatic Model Assessment of Insulin Resistance).

37. The method of any of claims 1-5, 8-11, and 15-36, wherein the one or more indicators selected from among Monitor Rating Scale (MRS), Mystical Experience Questionnaire (MEQ30), and Challenging Experiences Questionnaire (CEQ).

38. The method of any of claims 1-5, 8-11, and 15-37, wherein the one or more indicators selected from among vital signs, blood chemistry and hematology tests, urinalysis, electrocardiograms (ECG), and Columbia-Suicide Severity Rating Scale (C-SSRS).

39. The method of claim 38, wherein the blood chemistry and hematology tests comprise measuring the levels of one or more of Na⁺, K⁺, Cl⁻, HCO₃⁻, Ca⁺⁺, Mg⁺⁺, P, BUN, creatinine, glucose, total bilirubin, albumin, ALT, AST, GGT, CK, LDH, alkaline phosphatase, complete blood count, white cell differential count and platelet count.

40. The method of claim 38, wherein the urinalysis comprises measuring one or more of pH, specific gravity, protein, occult blood, glucose, and ketones plus microscopic examination of sediment for RBC, WBC, epithelial cells, casts, crystals, and bacteria.

41. The method of any of claims 1-5, 8-11, and 15-40, wherein the one or more indicators are measured during one or more of the integration sessions.

42. The method of any of claims 11-41, wherein at least one of the one or more parameters is presence of gastrointestinal disease and the subject meets the criteria for the parameter if the subject does not have a gastrointestinal disease that could interfere with absorption of orally-administered psilocybin or an active metabolite thereof.

43. The method of any of claims 11-42, wherein at least one of the one or more parameters is incidence of epilepsy and the subject meets the criteria for the parameter if the subject does not have epilepsy.

44. The method of any of claims 11-43, wherein at least one of the one or more parameters is incidence of schizophrenia spectrum or other psychotic disorders and the subject meets the criteria for the parameter if the subject does not have schizophrenia spectrum or other psychotic disorder that meets the DSM-5 criteria, major depressive disorder with psychotic features, or Bipolar I or Bipolar II Disorder.

45. The method of any of claims 11-44, wherein at least one of the one or more parameter is family history of psychosis, and the subject meets the criteria for the parameter if the subject does not have a family history of psychosis.

46. The method of any of claims 11-45, wherein at least one of the one or more parameters is a moderate or severe alcohol or drug use disorder, and the subject meets the criteria for the parameter if the subject does not have a moderate or severe alcohol or drug use disorder that meets the DSM-5 criteria.

47. The method of any of claims 11-46, wherein at least one of the one or more parameters is prior adverse effects from psilocybin or an active metabolite thereof, and the subject meets the criteria for the parameter if the subject does not have a prior adverse effects from psilocybin or an active metabolite thereof.

48. The method of any of claims 11-47, wherein at least one of the one or more parameters is use of prior or concomitant medications, and the subject meets the criteria for the parameter if the subject is not using any of the following prior or concomitant medications: UGT1A9 inhibitors, 1A10 inhibitors, regorafenib, rifampicin, phenytoin, eltrombopag, mefenamic acid, diflunisal, niflumic acid, sorafenib, isavuconazole, deferasirox, Ginseng, aldehyde or alcohol dehydrogenase inhibitors, disulfiram, amphetamines, buprenorphine, benzodiazepines, cocaine, methamphetamines, Ecstasy (MDMA), morphine, methadone, oxycodone, marijuana, ethyl glucuronide, fentanyl, tramadol, synthetic cannabinoids (K2) regular use of psychoactive prescription medication, opioids, tramadol, or benzodiazepines; concomitant use of antidepressants; regular use of medications having a primary centrally-acting serotonergic effect, monoamine oxidase inhibitors (MAOIs), or selective serotonin reuptake inhibitors (SSRIs), or use of serotonin-acting dietary supplements, 5-hydroxy-tryptophan, St. John's wort,

49. The method of any of claims 11-48, wherein the subject is identified for treatment with psilocybin or an active metabolite thereof if the subject meets the criteria for all of the parameters (1)-(10).

50. The method of any one of claims 1-5, 8-11, and 15-49, wherein the one or more indicators are measured at about 4 weeks and at about 12 weeks following administration of the one or more doses of psilocybin or an active metabolite thereof.

51. The method of any one of claims 1-5, 8-11, and 15-50, wherein the one or more indicators are measured at about 4 weeks, at about 8 weeks and at about 12 weeks following administration of the one or more doses of psilocybin or an active metabolite thereof.

52. The method of any one of claims 1-5, 8-11, and 15-51, wherein the one or more indicators are measured at about 4 weeks following administration of the one or more doses of psilocybin or an active metabolite thereof.

53. The method of any one of the claims 1-52, wherein the subject receiving treatment shows improvement in one or more symptoms of BED or one or more symptoms of BED are ameliorated.

54. The method of any one of the claims 1-53, wherein the subject receiving treatment shows improvements selected from among one or more of: decreased frequency of binge eating episodes, decreased Binge Eating Scale (BES), decreased binge eating episodes per day, decreased frequency of sense of loss of control over eating per day, decreased anxiety around food, decreased depression around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased overall anxiety, decreased anxiety regarding food, improved vision of self, improved self-confidence, improvement in depression and feeling genuinely happy, lack of binge eating desires, weight loss, improved eating behavior, decreased Hospital Anxiety and Depression Scale (HADS), decreased HADS—anxiety, decreased HADS—depression, decreased Patient Global Impression (PGI-I), and decreased Acceptance and Action Questionnaire (AAQ-II).

55. The method of any one of the claims 1-54, wherein the subject receiving treatment shows improvements selected from among one or more of: decreased frequency of binge eating episodes, decreased Binge Eating Scale (BES), decreased binge eating episodes per day, decreased anxiety around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased overall anxiety, decreased anxiety regarding food, improved vision of self, improved self-confidence, improvement in depression and feeling genuinely happy, lack of binge eating desires, weight loss, improved eating behavior, decreased Hospital Anxiety and Depression Scale (HADS), decreased Patient Global Impression (PGI-I), and decreased Acceptance and Action Questionnaire (AAQ-II).

56. The method of any one of the claims 1-55, wherein the subject receiving treatment shows improvements selected from among one or more of: decreased frequency of binge eating episodes, decreased Binge Eating Scale (BES), decreased binge eating episodes per day, decreased anxiety around food, decreased depression about eating behavior, decreased depression about control of weight, improved Clinician Global Impression—Improvement (CGI-I) scale, decreased interest in eating trigger foods, decreased waist circumference, decreased body mass index (BMI), decreased Hospital Anxiety and Depression Scale (HADS), decreased Patient Global Impression (PGI-I), and decreased Acceptance and Action Questionnaire (AAQ-II).

57. The method of any one of the claims 1-46, wherein the subject receiving treatment shows improvements selected from among one or more of: decreased overall anxiety, decreased anxiety regarding food, improved vision of self, improved self-confidence, improvement in depression and feeling genuinely happy, lack of binge eating desires, weight loss, and improved eating behavior.

58. The method of any of claims 1-57, wherein the one or more doses of psilocybin or an active metabolite thereof comprises 1, 2, 3, 4, or 5 doses of psilocybin or an active metabolite thereof.

59. The method of any of claims 1-58, wherein the one or more doses of psilocybin or an active metabolite thereof comprises 1 dose of psilocybin or an active metabolite thereof.

60. The method of any of claims 1-59, wherein the one or more doses of psilocybin or an active metabolite thereof comprises 2 doses of psilocybin or an active metabolite thereof.

61. The method of any of claims 1-60, wherein the dose is between at or about 10 mg and at or about 50 mg of psilocybin.

62. The method of claim 61, wherein the dose is at or about 25 mg of psilocybin.

63. The method of any one of claims 1-62, wherein the one or more integration session comprises 1, 2, 3, 4, or 5 integration sessions.

64. The method of any of claims 1-63, wherein the one or more integration session comprises 1 integration session.

65. The method of any of claims 1-64, wherein the one or more integration session comprises 2 integration sessions.

66. The method of any of claims 1-65, wherein the integration session is provided at or about 1 day after administering the dose of psilocybin or an active metabolite thereof.

67. The method of any of claims 1-66, further comprising providing one or more further integration sessions.

68. The method of claim 67, wherein the one or more further integration session is provided between about 6 days and at or about 9 days after administering the dose of psilocybin or an active metabolite thereof.

69. The method of claim 67 or 68, wherein the one or more further integration session is provided at or about 7 days after administering the dose of psilocybin or an active metabolite thereof.
